(19) United States
(12) Patent Application Publication
(10) Pub. No.: US 2019/0231751 A1
(43) Pub. Date: Aug. 1, 2019

(54) TREATMENT OF FIBROTIC DISORDERS WITH JUN N-TERMINAL KINASE INHIBITORS

(71) Applicant: Imago Pharmaceuticals, Inc., Jackson, WY (US)

(72) Inventors: Irene Griswold-Prenner, Jackson, WY (US); Karen Chen, New York, NY (US)

(21) Appl. No.: 16/332,334

(22) PCT Filed: Sep. 11, 2017
(86) PCT No.: PCT/US17/51014

§ 371 Date: Mar. 11, 2019

Related U.S. Application Data
(60) Provisional application No. 62/393,499, filed on Sep. 12, 2016.

(51) Int. Cl. A61K 31/4196; A61K 31/4192; A61K 31/444; A61K 31/4709; A61K 31/4725; A61K 31/498; A61K 31/421; A61K 31/4245; A61K 31/4178; A61K 31/4375; A61K 31/519; A61K 31/429; A61K 31/4365; A61K 31/5377; A61P 1/16; A61P 11/00; A61P 19/04

(52) U.S. Cl. CPC A61K 31/4196; A61P 19/04; A61K 31/444; A61K 31/4709; A61K 31/4725; A61K 31/498; A61K 31/421; A61K 31/4245; A61K 31/4178; A61K 31/4375; A61K 31/519; A61K 31/429; A61K 31/4365; A61K 31/5377; A61P 1/16; A61P 11/00; A61K 31/4192

(57) ABSTRACT

The present disclosure provides a method of treating diseases, conditions or disorders associated with fibrosis in a mammal using inhibitors of the Jun N-terminal kinase (JNK) pathway. The present disclosure in particular provides methods of treating diseases, conditions or disorders associated with fibrosis using a specific class of small molecule JNK inhibitors.

### CROSS-REFERENCE

This application claims benefit of U.S. Provisional Patent Application No. 62/393,499, filed Sep. 12, 2016, which is incorporated herein by reference in its entirety.

### FIELD OF THE INVENTION

This invention relates to compositions and assays for the use in treating diseases and disorders using neuroprotective agents.

### BACKGROUND OF THE INVENTION

In the following discussion certain articles and processes will be described for background and introductory purposes. Nothing contained herein is to be construed as an “admission” of prior art. Applicant expressly reserves the right to demonstrate, where appropriate, that the articles and processes referenced herein do not constitute prior art under the applicable statutory provisions

Mammalian cells respond to extracellular stimuli by activating signaling cascades that are mediated by members of the mitogen-activated protein (MAP) kinase family, which include the extracellular signal regulated kinases (ERKs), the p38 MAP kinases and the c-Jun N-terminal kinases (JNKs). Members of the JNK family are activated by pro-inflammatory cytokines, such as tumor necrosis factor-α (TNF-α) and interleukin-1 β (IL-1 β), as well as by environmental stress, including UV irradiation, hypoxia, and osmotic shock (see, e.g., Minden et al., Biochemica et Biophysica Acta 1997, 1333:F85-F104). Three distinct JNK genes, jnk1, jnk2 and jnk3 were identified and at least ten different splicing isoforms exist in mammalian cells (see, e.g., Gupta et al., EMBO J. 1996, 15:2760-2770).

Many effects of JNKs are mediated through phosphorylation of their target c-Jun that hetero- and homodimerizes with other Jun and Fos family members to form AP-1 transcription factors (Hibi M et al., Genes Dev. 1993; 7:2135-2148). In addition to their archetypical target c-Jun, JNKs also phosphorylate a number of other targets which is likely the basis for their involvement in a wide range of biological processes such as cell death, proliferation and inflammation (Bogoyevitch M A and Kobe B. Microbiol Mol Biol Rev. 2006; 70:1061-1095). Down-stream substrates of JNKs include transcription factors c-Jun, ATF-2, Elk1, p53 and a cell death domain protein (DENN) (see, e.g., Zhang et al. Proc. Natl. Acad. Sci. USA 1998, 95:2586-2591). Each JNK isoform binds to these substrates with different affinities, suggesting a regulation of signaling pathways by substrate specificity in vivo (Gupta et al., supra).

JNKs have been implicated in mediating a number of normal and pathophysiological responses. For example. JNKs have been implicated in mediating induction of fibrosis, including renal fibrosis (Ma F Y et al. J Am Soc Nephrol. 2007 February; 18(2):472-84), non-steatotic hepatic fibrosis (Aghazadeh S, Clin Nutr. 2010 June; 29(3):381-5; Schattenberg J M et al., Hepatology. 2006; 43:163-72; Farrell G C et al., Gut Liver. 2012 April; 6(2):149-71.), cardiac fibrosis (Yu Y et al., J Diabetes Res. 2016 Epub 2016 Jul. 31.) and lung fibrosis (Hashimoto S, American Journal of Respiratory and Critical Care Medicine, Vol. 163, No. 1 (2001), pp. 152-157; Shi-Wen X et al., Mol Cell Biol. 2006 July; 26(14):5518-27).

There is an unmet medical need for the development of potent, JNK specific inhibitors that are useful in treating the various conditions associated with JNK activation, such as fibrosis. The present disclosure addresses this need.

### SUMMARY OF THE INVENTION

The present disclosure provides a method of treating diseases, conditions or disorders associated with fibrosis in a mammal using inhibitors of the Jun N-terminal kinase (JNK) pathway. The present disclosure in particular provides methods of treating diseases, conditions or disorders associated with fibrosis using a specific class of JNK inhibitors as described in more detail herein.

In preferred embodiments, the preferred compounds for use in the methods of the present disclosure are disclosed in U.S. Pat. No. 8,450,363 to Sham et al., issued May 28, 2013, which is incorporated herein by reference in its entirety for all teachings. Methods of synthesis and in vitro activity of these compounds are likewise disclosed in U.S. Pat. No. 8,450,363, and are intended to be included herein for all purposes of practicing the invention.

Accordingly, in various aspects, the present disclosure provides for treatment of diseases, disorders and conditions associated with fibrosis in a mammal using a compound having a structure according to Formula (I):

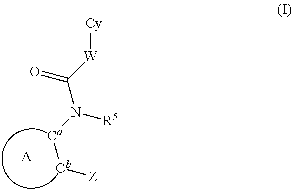

or a salt or solvate thereof, wherein ring A is 5-membered heteroaryl comprising a sulfur atom, wherein the heteroaryl is optionally substituted with 1 or 2 substituents independently chosen from alkyl, alkenyl, alkynyl, haloalkyl, heteroalkyl, C₃-C₁₀-cycloalkyl, 3- to 8-membered heterocycloalkyl, aryl, 5- or 6-membered heteroaryl, CN, halogen, OR¹², SR¹², NR¹²R¹³, C(O)R¹⁴, C(O)NR¹²R¹³, OC(O)NR¹²R¹³, C(O)OR¹², NR¹⁵C(O)R¹⁴, NR¹⁵C(O)OR¹², NR¹⁵C(O)NR¹²R¹³, NR¹⁵C(S)NR¹²R¹³, NR¹⁵S(O)₂R¹⁴, S(O)₂NR¹²R¹³, S(O)R¹⁴ and S(O)₂R¹⁴, wherein R¹², R¹³ and R¹⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹² and R¹³, together with the nitrogen atom to which they are bound form a 5- to 7-membered heterocyclic ring; and R¹⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; C^{a} and C^{b} are carbon atoms, which are adjacent to each other and are part of ring A; Z is 5- or 6-membered heteroaryl, with the proviso that (i) when ring A is thiophene, then Z is not a heteroaryl chosen from benzoimidazole, thiazole, and benzothiazole; (ii) when ring A is thiazole, then Z is not benzoimidazole; (iii) when ring A is thiophene, then Z is not substituted oxadiazole; and (iv) when ring A is thiophene, then Z is not pyrimidinone; R⁵ is chosen from H, acyl, substituted or unsubstituted C₁-C₆ alkyl, and C₃-C₆ cycloalkyl; W is chosen from C₁-C₄ alkylene, wherein the alkylene is optionally substituted with 1-4 substituents independently chosen from alkyl, alkenyl, alkynyl, haloalkyl, heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R⁴⁴, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R, and S(O)₂R⁴⁴, wherein R⁴², R⁴³ and R⁴⁵ are members independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁴² and R⁴³, together with the nitrogen atom to which they are bound are optionally joined to form a 5- to 7-membered heterocyclic ring; and R⁴⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; Cy is chosen from cycloalkyl, heterocycloalkyl, aryl, and heteroaryl, wherein the cycloalkyl, heterocycloalkyl, aryl or heteroaryl is optionally substituted with 1-6 substituents independently chosen from substituted or unsubstituted alkyl, substituted or unsubstituted alkenyl, substituted or unsubstituted alkynyl, haloalkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, CN, halogen, OR⁵², SR⁵², ═O, NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl.

In specific aspects, the present disclosure provides for treatment of diseases, disorders and conditions associated with fibrosis in a mammal using a compound having a structure according to Formula (I):

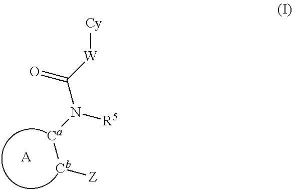

or a salt or solvate thereof, wherein ring A is 5-membered heteroaryl comprising a sulfur atom, wherein the heteroaryl is optionally substituted with 1 or 2 substituents independently chosen from alkyl, alkenyl, alkynyl, haloalkyl, heteroalkyl, C₃-C₁₀-cycloalkyl, 3- to 8-membered heterocycloalkyl, aryl, 5- or 6-membered heteroaryl, CN, halogen, OR¹², SR¹², NR¹²R¹³, C(O)R¹⁴, C(O)NR¹²R¹³, OC(O)NR¹²R¹³, C(O)OR¹², NR¹⁵C(O)R¹⁴, NR¹⁵C(O)OR¹², NR¹⁵C(O)NR¹²R¹³, NR¹⁵C(S)NR¹²R¹³, NR¹⁵S(O)₂R¹⁴, S(O)₂NR¹²R¹³, S(O)R¹⁴ and S(O)₂R¹⁴, wherein R¹², R¹³ and R¹⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹² and R¹³, together with the nitrogen atom to which they are bound form a 5- to 7-membered heterocyclic ring; and R¹⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; C^{a} and C^{b} are carbon atoms, which are adjacent to each other and are part of ring A; Z is triazole optionally substituted with alkyl, cycloalkyl, alkenyl, alkynyl, heteroalkyl, heterocycloalkyl, aryl, heteroaryl, —R^{a}, —OR^{a}, —SR^{a}, ═O, ═NR^{a}, ═N—OR^{a}, —NR^{a}R^{b}, -halogen, —SiR^{a}R^{b}R^{c}, —OC(O)R^{a}, —C(O)R^{e}, —C(O)OR^{a}, —C(O)NR^{a}R^{b}, —OC(O)NR^{a}R^{b}, —NR^{c}C(O)R^{e}, —NR^{c}C(O)NR^{a}R^{b}, —NR^{c}C(S)NR^{a}R^{b}, —NR^{c}C(O)OR^{a}, —NR^{c}C(NR^{a}R^{b})═NR^{d}, —S(O)R^{e}, —S(O)₂R^{e}, —S(O)₂NR^{a}R^{b}, —NR^{c}S(O)₂R^{a}, —CN, —NO₂, —N₃, —CH(Ph)₂, fluoro(C₁-C₄)alkoxy, and fluoro(C₁-C₄)alkyl, wherein R^{a}, R^{b}, R^{c}, R^{d} and R^{e} each independently hydrogen, C₁-C₂₄ alkyl, C₃-C₁₀ cycloalkyl, C₁-C₂₄ heteroalkyl, C₃-C₁₀ heterocycloalkyl, aryl, heteroaryl, arylalkyl and heteroarylalkyl; and wherein where two R groups are attached to the same nitrogen atom, they can be combined with the nitrogen atom to form a 5-, 6-, or 7-membered ring; R⁵ is chosen from H, acyl, substituted or unsubstituted C₁-C₆ alkyl, and C₃-C₆ cycloalkyl; W is chosen from C₁-C₄ alkylene, wherein the alkylene is optionally substituted with 1-4 substituents independently chosen from alkyl, alkenyl, alkynyl, haloalkyl, heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R⁴⁴, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R, and S(O)₂R⁴⁴, wherein R⁴², R⁴³ and R⁴⁵ are members independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁴² and R⁴³, together with the nitrogen atom to which they are bound are optionally joined to form a 5- to 7-membered heterocyclic ring; and R⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; Cy is chosen from cycloalkyl, heterocycloalkyl, aryl, and heteroaryl, wherein the cycloalkyl, heterocycloalkyl, aryl or heteroaryl is optionally substituted with 1-6 substituents independently chosen from substituted or unsubstituted alkyl, substituted or unsubstituted alkenyl, substituted or unsubstituted alkynyl, haloalkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, CN, halogen, OR⁵², SR⁵², ═O, NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; wherein the aryl groups are chosen from 5-, 6- or 7-membered, aromatic carbocyclic group having a single ring or being fused to other aromatic or non-aromatic rings; and wherein the heteroaryl groups are chosen from polyunsaturated, 5-, 6- or 7-membered aromatic moiety containing at least one heteroatom chosen from N, O, S, Si and B, wherein the nitrogen and sulphur atoms are optionally oxidized, and the nitrogen atom(s) are optionally quarternized, and wherein the heteroaryl groups can be a single ring or be fused to other aryl, heteroaryl, cycloalkyl or heterocycloalkyl rings.

The present disclosure further provides for a pharmaceutical composition for the treatment or prevention of a disease, disorder or condition associated with fibrosis comprising a compound according to Formula (I) and a pharmaceutically acceptable carrier.

The present disclosure also provides for treatment of diseases, disorders and conditions associated with fibrosis in a mammal using a compound having a structure according to Formula (VIII):

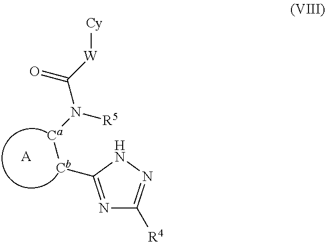

or a tautomer, mixture of tautomers, salt or solvate thereof, wherein ring A is 5- or 6-membered heteroaryl, wherein the heteroaryl is optionally substituted with 1-3 substituents independently chosen from alkyl, alkenyl, alkynyl, haloalkyl, heteroalkyl, C₃-C₁₀-cycloalkyl, 3- to 8-membered heterocycloalkyl, aryl, 5- or 6-membered heteroaryl, CN, halogen, OR¹², SR¹², NR¹²R¹³, C(O)R¹⁴, C(O)NR¹²R¹³, OC(O)NR¹²R¹³, C(O)OR¹², NR¹⁵C(O)R¹⁴, NR¹⁵C(O)OR¹², NR¹⁵C(O)NR¹²R¹³, NR¹⁵C(S)NR¹²R¹³, NR¹⁵S(O)₂R¹⁴, S(O)₂NR¹²R¹³, S(O)R¹⁴ and S(O)₂R¹⁴, wherein R¹², R¹³ and R¹⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹² and R¹³, together with the nitrogen atom to which they are bound form a 5- to 7-membered heterocyclic ring; and R¹⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; C^{a} and C^{b} are carbon atoms, which are adjacent to each other and, which are part of ring A; R⁴ is chosen from H, independently chosen from H, C₁-C₄ alkyl, C₁-C₄ alkenyl, C₁-C₄ alkynyl, C₁-C₄ haloalkyl, C₃-C₆ cycloalkyl, 3- to 6-membered heterocycloalkyl, aryl, and 5- or 6-membered heteroaryl, CN, halogen, OR¹⁷, SR¹⁷ and NR¹⁷R¹⁸, wherein R¹⁷ and R¹⁸ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹⁷ and R¹⁸, together with the nitrogen atom to which they are bound form a 5- to 7-membered heterocyclic ring; R⁵ is chosen from H, acyl, C₁-C₆ alkyl, and C₃-C₆ cycloalkyl; W is chosen from C₁-C₄ alkylene, wherein the alkylene is optionally substituted with from 1 to 4 substituents chosen from alkyl, alkenyl, alkynyl, haloalkyl, heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R⁴⁴, and S(O)₂R⁴⁴, wherein R⁴², R⁴³ and R⁴⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁴² and R⁴³, together with the nitrogen atom to which they are bound are optionally joined to form a 5- to 7-membered heterocyclic ring; and R⁴⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; Cy is chosen from cycloalkyl, heterocycloalkyl, aryl, and heteroaryl, wherein the cycloalkyl, heterocycloalkyl, aryl or heteroaryl is optionally substituted with 1-6 substituents independently chosen from substituted or unsubstituted alkyl, substituted or unsubstituted alkenyl, substituted or unsubstituted alkynyl, haloalkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, CN, halogen, OR⁵², SR⁵², NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl.

The present disclosure further provides for a pharmaceutical composition for the treatment or prevention of a disease, disorder or condition associated with fibrosis comprising a compound according to Formula (VIII) and a pharmaceutically acceptable carrier.

These aspects and other features and advantages of the invention are described below in more detail. Those skilled in the art will recognize, or be able to ascertain using no more than routine experimentation, many equivalents to the specific embodiments of the invention described herein. Such equivalents are intended to be encompassed by the following claims.

### BRIEF DESCRIPTION OF THE FIGURES

FIG. 1 shows exemplary individual compounds that can be used in the methods of the present disclosure.

FIG. 2 is a graph showing the plasma levels of IGP001 and IGP002 detected in the rats treated with these compounds.

FIG. 3 is a graph illustrating the reduction of c-Jun phosphorylation in PMBCs following treatment of rats with IGP001 and IGP002.

FIG. 4 is a graph showing the ability of 10 mg/kg IGP001 (“B”) treatment versus positive (ZVAD) and negative (Control) controls (Female) in prevention of lung fibrosis in a mouse bleomycin-induced fibrosis model.

FIG. 5 are histology photos showing 10 and 30 mg/kg IGP001 treatment versus positive (ZVAD) and negative (No Compound and CTL) controls in a mouse bleomycin-induced idiopathic pulmonary fibrosis (“IPF”) model.

FIG. 6 is a graph illustrating the effect of 30 mg/kg IGP001 in mouse bleomycin-induced IPF model.

### DETAILED DESCRIPTION OF THE DISCLOSURE

The practice of the techniques described herein may employ, unless otherwise indicated, conventional techniques and descriptions molecular biology (including recombinant techniques), cell biology, biochemistry, and genetic engineering technology, which are within the skill of those who practice in the art. Such conventional techniques and descriptions can be found in standard laboratory manuals such as Green and Sambrook, Molecular Cloning: A Laboratory Manual. 4th, ed., Cold Spring Harbor Laboratory Press, Cold Spring Harbor, N.Y., (2014); Current Protocols in Molecular Biology, Ausubel, et al. eds., (2017); Neumann, et al., Electroporation and Electrofusion in Cell Biology, Plenum Press, New York, 1989; and Chang, et al., Guide to Electroporation and Electrofusion, Academic Press, California (1992), all of which are herein incorporated in their entirety by reference for all purposes.

Note that as used herein and in the appended claims, the singular forms “a,” “an,” and “the” include plural referents unless the context clearly dictates otherwise. Thus, for example, reference to “a cell” refers to one or more cells, and reference to “the system” includes reference to equivalent steps, methods and devices known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in smaller ranges, and are also encompassed within the invention, subject to any specifically excluded limit in the stated range. Where the stated range includes one or both of the limits, ranges excluding either or both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention. The terms used herein are intended to have the plain and ordinary meaning as understood by those of ordinary skill in the art.

### Definitions

The definitions and explanations below are for the terms as used throughout this entire document including both the specification and the claims. Throughout the specification and the appended claims, a given formula or name shall encompass all isomers thereof, such as stereoisomers, geometrical isomers, optical isomers, tautomers, and mixtures thereof where such isomers exist, as well as pharmaceutically acceptable salts and solvates thereof, such as hydrates.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Where multiple substituents are indicated as being attached to a structure, those substituents are independently chosen. For example “ring A is optionally substituted with 1, 2 or 3 R_{q} groups” indicates that ring A is substituted with 1, 2 or 3 R_{q} groups, wherein the R_{q} groups are independently chosen (i.e., can be the same or different).

Compounds were named using Autonom 2000 4.01.305, which is available from Beilstein Information Systems, Inc, Englewood, Colo.; ChemDraw v. 10.0, (available from Cambridgesoft at 100 Cambridge Park Drive, Cambridge, Mass. 02140), or ACD Name pro, which is available from Advanced Chemistry Development, Inc., at 110 Yonge Street, 14^{th} floor, Toronto, Ontario, Canada M5c 1T4. Alternatively, the names were generated based on the IUPAC rules or were derived from names originally generated using the aforementioned nomenclature programs. A person of skill in the art will appreciate that chemical names for tautomeric forms of the current compounds will vary slightly, but will nevertheless describe the same compound. For example, the names N-(2-(3-methyl-1H-1,2,4-triazol-5-yl)thiophen-3-yl)-2-(naphthalen-1-yl)acetamide and N-(2-(5-methyl-4H-1,2,4-triazol-3-yl)thiophen-3-yl)-2-(naphthalen-1-yl)acetamide describe two tautomeric forms of the same compound.

Where substituent groups are specified by their conventional chemical formulae, written from left to right, they equally encompass the chemically identical substituents, which would result from writing the structure from right to left. For example, “—CH₂O—” is intended to also recite “—OCH₂—”.

The term “alkyl,” by itself or as part of another substituent, means, unless otherwise stated, a straight or branched chain hydrocarbon radical having the number of carbon atoms designated (e.g., C₁-C₁0 means one to ten carbon atoms). Typically, an alkyl group will have from 1 to 24 carbon atoms, for example having from 1 to 10 carbon atoms, from 1 to 8 carbon atoms or from 1 to 6 carbon atoms. A “lower alkyl” group is an alkyl group having from 1 to 4 carbon atoms. The term “alkyl” includes di- and multivalent radicals. For example, the term “alkyl” includes “alkylene” wherever appropriate, e.g., when the formula indicates that the alkyl group is divalent or when substituents are joined to form a ring. Examples of alkyl radicals include, but are not limited to, methyl, ethyl, n-propyl, iso-propyl, n-butyl, tert-butyl, iso-butyl, sec-butyl, as well as homologs and isomers of, for example, n-pentyl, n-hexyl, n-heptyl and n-octyl.

The term “alkylene” by itself or as part of another substituent means a divalent (diradical) alkyl group, wherein alkyl is defined herein. “Alkylene” is exemplified, but not limited, by —CH₂CH₂CH₂CH₂—. Typically, an “alkylene” group will have from 1 to 24 carbon atoms, for example, having 10 or fewer carbon atoms (e.g., 1 to 8 or 1 to 6 carbon atoms). A “lower alkylene” group is an alkylene group having from 1 to 4 carbon atoms.

The term “alkenyl” by itself or as part of another substituent refers to a straight or branched chain hydrocarbon radical having from 2 to 24 carbon atoms and at least one double bond. A typical alkenyl group has from 2 to 10 carbon atoms and at least one double bond. In one embodiment, alkenyl groups have from 2 to 8 carbon atoms or from 2 to 6 carbon atoms and from 1 to 3 double bonds. Exemplary alkenyl groups include vinyl, 2-propenyl, 1-but-3-enyl, crotyl, 2-(butadienyl), 2,4-pentadienyl, 3-(1,4-pentadienyl), 2-isopentenyl, 1-pent-3-enyl, 1-hex-5-enyl and the like.

The term “alkynyl” by itself or as part of another substituent refers to a straight or branched chain, unsaturated or polyunsaturated hydrocarbon radical having from 2 to 24 carbon atoms and at least one triple bond. A typical “alkynyl” group has from 2 to 10 carbon atoms and at least one triple bond. In one aspect of the disclosure, alkynyl groups have from 2 to 6 carbon atoms and at least one triple bond. Exemplary alkynyl groups include prop-1-ynyl, prop-2-ynyl (i.e., propargyl), ethynyl and 3-butynyl.

The terms “alkoxy,” “alkylamino” and “alkylthio” (or thioalkoxy) are used in their conventional sense, and refer to alkyl groups that are attached to the remainder of the molecule via an oxygen atom, an amino group, or a sulfur atom, respectively.

The term “heteroalkyl,” by itself or in combination with another term, means a stable, straight or branched chain hydrocarbon radical consisting of the stated number of carbon atoms (e.g., C₂-C₁0, or C₂-C₈) and at least one heteroatom chosen, e.g., from N, O, S, Si, B and P (in one embodiment, N, O and S), wherein the nitrogen, sulfur and phosphorus atoms are optionally oxidized, and the nitrogen atom(s) are optionally quaternized. The heteroatom(s) is/are placed at any interior position of the heteroalkyl group. Examples of heteroalkyl groups include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂—S(O)—CH₃, —CH₂—CH₂—S(O)₂—CH₃, —CH.dbd.CH—O—CH₃, —CH₂—Si(CH₃)₃, —CH₂—CH.dbd.N—OCH₃, and —CH.dbd.CH—N(CH₃)—CH₃. Up to two heteroatoms can be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. Typically, a heteroalkyl group will have from 3 to 24 atoms (carbon and heteroatoms, excluding hydrogen) (3- to 24-membered heteroalkyl). In another example, the heteroalkyl group has a total of 3 to 10 atoms (3- to 10-membered heteroalkyl) or from 3 to 8 atoms (3- to 8-membered heteroalkyl). The term “heteroalkyl” includes “heteroalkylene” wherever appropriate, e.g., when the formula indicates that the heteroalkyl group is divalent or when substituents are joined to form a ring.

The term “cycloalkyl” by itself or in combination with other terms, represents a saturated or unsaturated, non-aromatic carbocyclic radical having from 3 to 24 carbon atoms, for example, having from 3 to 12 carbon atoms (e.g., C₃-C₈ cycloalkyl or C₃-C₆ cycloalkyl). Examples of cycloalkyl include, but are not limited to, cyclopropyl, cyclobutyl, cyclopentyl, cyclohexyl, cycloheptyl, 1-cyclohexenyl, 3-cyclohexenyl, cycloheptyl and the like. The term “cycloalkyl” also includes bridged, polycyclic (e.g., bicyclic) structures, such as norbornyl, adamantyl and bicyclo[2.2.1]heptyl. The “cycloalkyl” group can be fused to at least one (e.g., 1 to 3) other ring chosen from aryl (e.g., phenyl), heteroaryl (e.g., pyridyl) and non-aromatic (e.g., carbocyclic or heterocyclic) rings. When the “cycloalkyl” group includes a fused aryl, heteroaryl or heterocyclic ring, then the “cycloalkyl” group is attached to the remainder of the molecule via the carbocyclic ring.

The term “heterocycloalkyl”, “heterocyclic”, “heterocycle”, or “heterocyclyl”, by itself or in combination with other terms, represents a carbocyclic, non-aromatic ring (e.g., 3- to 8-membered ring and for example, 4-, 5-, 6- or 7-membered ring) containing at least one and up to 5 heteroatoms chosen from, e.g., N, O, S, Si, B and P (for example, N, O and S), wherein the nitrogen, sulfur and phosphorus atoms are optionally oxidized, and the nitrogen atom(s) are optionally quaternized (e.g., from 1 to 4 heteroatoms chosen from nitrogen, oxygen and sulfur), or a fused ring system of 4- to 8-membered rings, containing at least one and up to 10 heteroatoms (e.g., from 1 to 5 heteroatoms chosen from N, O and S) in stable combinations known to those of skill in the art. Exemplary heterocycloalkyl groups include a fused phenyl ring. When the “heterocyclic” group includes a fused aryl, heteroaryl or cycloalkyl ring, then the “heterocyclic” group is attached to the remainder of the molecule via a heterocycle. A heteroatom can occupy the position at which the heterocycle is attached to the remainder of the molecule. Exemplary heterocycloalkyl or heterocyclic groups of the present disclosure include morpholinyl, thiomorpholinyl, thiomorpholinyl S-oxide, thiomorpholinyl S,S-dioxide, piperazinyl, homopiperazinyl, pyrrolidinyl, pyrrolinyl, imidazolidinyl, tetrahydropyranyl, piperidinyl, tetrahydrofuranyl, tetrahydrothienyl, piperidinyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazolyl, dihydropyridyl, dihydropyrimidinyl, dihydrofuryl, dihydropyranyl, tetrahydrothienyl S-oxide, tetrahydrothienyl S,S-dioxide, homothiomorpholinyl S-oxide, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

By “aryl” is meant a 5-, 6- or 7-membered, aromatic carbocyclic group having a single ring (e.g., phenyl) or being fused to other aromatic or non-aromatic rings (e.g., from 1 to 3 other rings). When the “aryl” group includes a non-aromatic ring (such as in 1,2,3,4-tetrahydronaphthyl) or heteroaryl group then the “aryl” group is linked to the remainder of the molecule via an aryl ring (e.g., a phenyl ring). The aryl group is optionally substituted (e.g., with 1 to 5 substituents described herein). In one example, the aryl group has from 6 to 10 carbon atoms. Non-limiting examples of aryl groups include phenyl, 1-naphthyl, 2-naphthyl, quinoline, indanyl, indenyl, dihydronaphthyl, fluorenyl, tetralinyl, benzo[d][1,3]dioxolyl or 6,7,8,9-tetrahydro-5H-benzo[a]cycloheptenyl. In one embodiment, the aryl group is chosen from phenyl, benzo[d][1,3]dioxolyl and naphthyl. The aryl group, in yet another embodiment, is phenyl.

The term “arylalkyl” is meant to include those radicals in which an aryl group or heteroaryl group is attached to an alkyl group to create the radicals -alkyl-aryl and -alkyl-heteroaryl, wherein alkyl, aryl and heteroaryl are defined herein. Exemplary “arylalkyl” groups include benzyl, phenethyl, pyridylmethyl and the like.

By “aryloxy” is meant the group —O-aryl, where aryl is as defined herein. In one example, the aryl portion of the aryloxy group is phenyl or naphthyl. The aryl portion of the aryloxy group, in one embodiment, is phenyl.

The term “heteroaryl” or “heteroaromatic” refers to a polyunsaturated, 5-, 6- or 7-membered aromatic moiety containing at least one heteroatom (e.g., 1 to 5 heteroatoms, such as 1-3 heteroatoms) chosen from N, O, S, Si and B (for example, N, O and S), wherein the nitrogen and sulfur atoms are optionally oxidized, and the nitrogen atom(s) are optionally quaternized. The “heteroaryl” group can be a single ring or be fused to other aryl, heteroaryl, cycloalkyl or heterocycloalkyl rings (e.g., from 1 to 3 other rings). When the “heteroaryl” group includes a fused aryl, cycloalkyl or heterocycloalkyl ring, then the “heteroaryl” group is attached to the remainder of the molecule via the heteroaryl ring. A heteroaryl group can be attached to the remainder of the molecule through a carbon- or heteroatom. In one example, the heteroaryl group has from 4 to 10 carbon atoms and from 1 to 5 heteroatoms chosen from O, S and N. Non-limiting examples of heteroaryl groups include pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridyl, benzotetrahydrofuranyl, benzotetrahydrothienyl, purinyl, benzodioxolyl, triazinyl, pteridinyl, benzothiazolyl, imidazopyridyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Exemplary heteroaryl groups include imidazolyl, pyrazolyl, thiadiazolyl, triazolyl, isoxazolyl, isothiazolyl, imidazolyl, thiazolyl, oxadiazolyl, and pyridyl. Other exemplary heteroaryl groups include 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, pyridin-4-yl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are chosen from the group of acceptable aryl group substituents described below.

For brevity, the term “aryl” when used in combination with other terms (e.g., aryloxy, arylthioxy, arylalkyl) includes both aryl and heteroaryl rings as defined above.

Each of the above terms (e.g., “alkyl”, “cycloalkyl”, “heteroalkyl”, heterocycloalkyl”, “aryl” and “heteroaryl”) are meant to include both substituted and unsubstituted forms of the indicated radical. The term “substituted” for each type of radical is explained below. When a compound of the present disclosure includes more than one substituent, then each of the substituents is independently chosen.

The term “substituted” in connection with alkyl, alkenyl, alkynyl, cycloalkyl, heteroalkyl and heterocycloalkyl radicals (including those groups referred to as alkylene, heteroalkylene, heteroalkenyl, cycloalkenyl, heterocycloalkenyl, and the like) refers to one or more substituents, wherein each substituent is independently chosen from, but not limited to, 3- to 10-membered heteroalkyl, C₃-C₁₀ cycloalkyl, 3- to 10-membered heterocycloalkyl, aryl, heteroaryl, —OR^{a}, —SR^{a}, .dbd.O, .dbd.NR^{a}, .dbd.N—OR^{a}, —NR^{a}R^{b}, -halogen, —SiR^{a}R^{b}R^{c}, —OC(O)R^{a}, —C(O)R^{e}, —C(O)OR^{a}, —C(O)NR^{a}R^{b}, —OC(O)NR^{a}R^{b}, —NR^{c}C(O)R^{e}, —NR^{c}C(O)NR^{a}R^{b}, —NR^{c}C(S)NR^{a}R^{b}, —NR^{c}C(O)OR^{a}, —NR^{c}C(NR^{a}R^{b}).dbd.NR^{d}, —S(O)R^{e}, —S(O)₂R^{e}, —S(O)₂NR^{a}R^{b}, —NR^{c}S(O)₂R^{a}, —CN and —NO₂. R^{a}, R^{b}, R^{c}, R^{d} and R^{e} each independently refer to hydrogen, C₁-C₂4 alkyl (e.g., C₁-C₁0 alkyl or C₁-C₆ alkyl), C₃-C₁0 cycloalkyl, C₁-C₂4 heteroalkyl (e.g., C₁-C₁0 heteroalkyl or C₁-C₆ heteroalkyl), C₃-C₁0 heterocycloalkyl, aryl, heteroaryl, arylalkyl and heteroarylalkyl, wherein, in one embodiment, R^{e} is not hydrogen. When two of the above R groups (e.g., R^{a} and R^{b}) are attached to the same nitrogen atom, they can be combined with the nitrogen atom to form a 5-, 6-, or 7-membered ring. For example, —NR^{a}R^{b} is meant to include pyrrolidinyl, N-alkyl-piperidinyl and morpholinyl.

The term “substituted” in connection with aryl and heteroaryl groups, refers to one or more substituents, wherein each substituent is independently chosen from, but not limited to, alkyl (e.g., C₁-C₂4 alkyl, C₁-C₁0 alkyl or C₁-C₆ alkyl), cycloalkyl (e.g., C₃-C₁0 cycloalkyl, or C₃-C₈ cycloalkyl), alkenyl (e.g., C₁-C₁0 alkenyl or C₁-C₆ alkenyl), alkynyl (e.g., C₁-C₁0 alkynyl or C₁-C₆ alkynyl), heteroalkyl (e.g., 3- to 10-membered heteroalkyl), heterocycloalkyl (e.g., C₃-C₈ heterocycloalkyl), aryl, heteroaryl, —R^{a}, —OR^{a}, —SR^{a}, .dbd.O, .dbd.NR^{a}, .dbd.N—OR^{a}, —NR^{a}R^{b}, -halogen, —SiR^{a}R^{b}R^{c}, —OC(O)R^{a}, —C(O)R^{e}, —C(O)OR^{a}, —C(O)NR^{a}R^{b}, —OC(O)NR^{a}R^{b}, —NR^{c}C(O)R^{e}, —NR^{c}C(O)NR^{a}R^{b}, —NR^{c}C(S)NR^{a}R^{b}, —NR^{c}C(O)OR^{a}, —NR^{c}C(NR^{a}R^{b}).dbd.NR^{d}, —S(O)R^{e}, —S(O)₂R^{e}, —S(O)₂NR^{a}R^{b}, —NR^{c}S(O)₂R^{a}, —CN, —NO₂, —N₃, —CH(Ph)₂, fluoro(C₁-C₄)alkoxy, and fluoro(C₁-C₄)alkyl, in a number ranging from zero to the total number of open valences on the aromatic ring system, wherein R^{a}, R^{b}, R^{c}, R^{d} and R^{e} each independently refer to hydrogen, C₁-C₂4 alkyl (e.g., C₁-C₁0 alkyl or C₁-C₆ alkyl), C₃-C₁₀ cycloalkyl, C₁-C₂4 heteroalkyl (e.g., C₁-C₁₀ heteroalkyl or C₁-C₆ heteroalkyl), C₃-C₁₀ heterocycloalkyl, aryl, heteroaryl, arylalkyl and heteroarylalkyl, wherein, in one embodiment, R^{e} is not hydrogen. When two R groups (e.g., R^{a} and R^{b}) are attached to the same nitrogen atom, they can be combined with the nitrogen atom to form a 5-, 6-, or 7-membered ring. For example, —NR^{a}R^{b} is meant to include pyrrolidinyl, N-alkyl-piperidinyl and morpholinyl.

The term “substituted” in connection with aryl and heteroaryl groups also refers to one or more fused ring(s), in which two hydrogen atoms on adjacent atoms of the aryl or heteroaryl ring are optionally replaced with a substituent of the formula -T-C(O)—(CRR′)_{q}—U—, wherein T and U are independently —NR—, —O—, —CRR′— or a single bond, and q is an integer from 0 to 3. Alternatively, two of the hydrogen atoms on adjacent atoms of the aryl or heteroaryl ring can optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer from 1 to 4. One of the single bonds of the ring so formed can optionally be replaced with a double bond. Alternatively, two of the hydrogen atoms on adjacent atoms of the aryl or heteroaryl ring can optionally be replaced with a substituent of the formula —(CRR′)ₛ—X—(CR″R′″)_{d}—, where s and d are independently integers from 0 to 3, and X is —O—, —NR′—, —S—, —S(O)—, —S(O)₂—, or —S(O)₂NR′—, wherein the substituents R, R′, R″ and R′″ in each of the formulas above are independently chosen from hydrogen and (C₁-C₆)alkyl.

The terms “halo” or “halogen,” by themselves or as part of another substituent, mean at least one of fluorine, chlorine, bromine and iodine.

By “haloalkyl” is meant an alkyl radical, wherein alkyl is as defined above and wherein at least one hydrogen atom is replaced by a halogen atom. The term “haloalkyl,” is meant to include monohaloalkyl and polyhaloalkyl. For example, the term “halo(C₁-C₄)alkyl” is mean to include, but not limited to, chloromethyl, 1-bromoethyl, fluoromethyl, difluoromethyl, trifluoromethyl, 1,1,1-trifluoroethyl and 4-chlorobutyl, 3-bromopropyl.

As used herein, the term “acyl” describes the group —C(O)R^{e}, wherein R^{e} is chosen from hydrogen, C₁-C₂4 alkyl (e.g., C₁-C₁₀ alkyl or C₁-C₆ alkyl), C₁-C₂4 alkenyl (e.g., C₁-C₁0 alkenyl or C₁-C₆ alkenyl), C₁-C₂4 alkynyl (e.g., C₁-C₁0 alkynyl or C₁-C₆ alkynyl), C₃-C₁0 cycloalkyl, C₁-C₂4 heteroalkyl (e.g., C₁-C₁0 heteroalkyl or C₁-C₆ heteroalkyl), C₃-C₁0 heterocycloalkyl, aryl, heteroaryl, arylalkyl and heteroarylalkyl. In one embodiment, R^{e} is not hydrogen.

By “alkanoyl” is meant an acyl radical —C(O)-Alk-, wherein Alk is an alkyl radical as defined herein. Examples of alkanoyl include acetyl, propionyl, butyryl, isobutyryl, valeryl, isovaleryl, 2-methyl-butyryl, 2,2-dimethylpropionyl, hexanoyl, heptanoyl, octanoyl and the like.

As used herein, the term “heteroatom” includes oxygen (O), nitrogen (N), sulfur (S), silicon (Si), boron (B) and phosphorus (P). In one embodiment, heteroatoms are O, S and N.

By “oxo” is meant the group ═O.

The symbol “R” is a general abbreviation that represents a substituent group as described herein. Exemplary substituent groups include alkyl, alkenyl, alkynyl, cycloalkyl, heteroalkyl, aryl, heteroaryl and heterocycloalkyl groups, each as defined herein.

As used herein, the term “aromatic ring” or “non-aromatic ring” is consistent with the definition commonly used in the art. For example, aromatic rings include phenyl and pyridyl. Non-aromatic rings include cyclohexanes.

As used herein, the term “fused ring system” means at least two rings, wherein each ring has at least 2 atoms in common with another ring. “Fused ring systems can include aromatic as well as non-aromatic rings. Examples of “fused ring systems” are naphthalenes, indoles, quinolines, chromenes and the like. Likewise, the term “fused ring” refers to a ring that has at least two atoms in common with the ring to which it is fused.

The phrase “therapeutically effective amount” as used herein means that amount of a compound, material, or composition of the present disclosure, which is effective for producing a desired therapeutic effect, at a reasonable benefit/risk ratio applicable to any medical treatment. For example, a “therapeutically effective amount” is an amount effective to reduce or lessen at least one symptom of the disease or condition being treated or to reduce or delay onset of one or more clinical markers or symptoms associated with the disease or condition, or to modify or reverse the disease process.

The terms “treatment” or “treating” when referring to a disease or condition, means producing a desired therapeutic effect. Exemplary therapeutic effects include delaying onset or reducing at least one symptom associated with the disease, positively affecting (e.g., reducing or delaying onset) a clinical marker associated with the disease and slowing or reversing disease progression.

The term “pharmaceutically acceptable” refers to those properties and/or substances that are acceptable to a patient (e.g., human patient) from a toxicological and/or safety point of view.

The term “pharmaceutically acceptable salts” means salts of the compounds of the present disclosure, which may be prepared with relatively nontoxic acids or bases, depending on the particular substituents found on the compounds described herein. When compounds of the present disclosure contain relatively acidic functionalities (e.g., —COOH group), base addition salts can be obtained by contacting the compound (e.g., neutral form of such compound) with a sufficient amount of the desired base, either neat or in a suitable inert solvent. Examples of pharmaceutically acceptable base addition salts include lithium, sodium, potassium, calcium, ammonium, organic amino, magnesium and aluminum salts and the like. When compounds of the present disclosure contain relatively basic functionalities (e.g., amines), acid addition salts can be obtained, e.g., by contacting the compound (e.g., neutral form of such compound) with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, diphosphoric, monohydrogenphosphoric, dihydrogenphosphoric, sulfuric, monohydrogensulfuric, hydriodic and the like, as well as the salts derived from relatively nontoxic organic acids like formic, acetic, propionic, isobutyric, malic, maleic, malonic, benzoic, succinic, suberic, fumaric, lactic, mandelic, phthalic, benzenesulfonic, p-tolylsulfonic, citric, tartaric, methanesulfonic, 2-hydroxyethylsulfonic, salicylic, stearic and the like. Also included are salts of amino acids such as arginate and the like, and salts of organic acids like glucuronic or galactunoric acids and the like (see, for example, Berge et al., Journal of Pharmaceutical Science, 1977, 66: 1-19). Certain specific compounds of the present disclosure contain both, basic and acidic, functionalities that allow the compounds to be converted into either base or acid addition salts.

The neutral forms of the compounds can be regenerated, for example, by contacting the salt with a base or acid and isolating the parent compound in the conventional manner. The parent form of the compound can differ from the various salt forms in certain physical properties, such as solubility in polar solvents, but otherwise the salts are equivalent to the parent form of the compound for the purposes of the present disclosure.

When a substituent includes a negatively charged oxygen atom “O⁻”, e.g., in “—COO⁻”, then the formula is meant to optionally include a proton or an organic or inorganic cationic counterion (e.g., Na+). In one example, the resulting salt form of the compound is pharmaceutically acceptable. Further, when a compound of the present disclosure includes an acidic group, such as a carboxylic acid group, e.g., written as the substituent “—COOH”, “—CO₂H” or “—C(O)₂H”, then the formula is meant to optionally include the corresponding “de-protonated” form of that acidic group, e.g., “—COO⁻”, “—CO₂⁻” or “—C(O)₂⁻”, respectively.

In addition to salt forms, the present disclosure provides use of compounds which are in a prodrug form. Prodrugs of the compounds described herein are those compounds that readily undergo chemical changes under physiological conditions to provide the compounds of the present disclosure. Non-limiting examples of “pharmaceutically acceptable derivative” or “prodrug” include pharmaceutically acceptable esters, phosphate esters or sulfonate esters thereof as well as other derivatives of a compound of this present disclosure which, upon administration to a recipient, is capable of providing, either directly or indirectly, a compound of this present disclosure. In one embodiment, derivatives or prodrugs are those that increase the bioavailability of the compounds of this present disclosure when such compounds are administered to a mammal (e.g., by allowing an orally administered compound to be more readily absorbed into the blood stream) or which enhance delivery of the parent compound to a biological compartment (e.g., the brain or lymphatic system) relative to the parent species.

Prodrugs include a variety of esters (i.e., carboxylic acid ester). Ester groups, which are suitable as prodrug groups are generally known in the art and include benzyloxy, di(C₁-C₆)alkylaminoethyloxy, acetoxymethyl, pivaloyloxymethyl, phthalidoyl, ethoxycarbonyloxyethyl, 5-methyl-2-oxo-1,3-dioxol-4-yl methyl, and (C₁-C₆)alkoxy esers, optionally substituted by N-morpholino and amide-forming groups such as di(C₁-C₆)alkylamino. For example, ester prodrug groups include C₁-C₆ alkoxy esters. Those skilled in the art will recognize various synthetic methodologies that may be employed to form pharmaceutically acceptable prodrugs of the compounds of the present disclosure (e.g., via esterification of a carboxylic acid group).

In an exemplary embodiment, the prodrug is suitable for treatment/prevention of those diseases and conditions that require the drug molecule to cross the blood brain barrier. In one embodiment, the prodrug enters the brain, where it is converted into the active form of the drug molecule. In another example, a prodrug is used to enable an active drug molecule to reach the inside of the eye after topical application of the prodrug to the eye. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure can exist in multiple crystalline or amorphous forms (“polymorphs”). In general, all physical forms are of use in the methods contemplated by the present disclosure and are intended to be within the scope of the present disclosure. “Compound or a pharmaceutically acceptable salt, hydrate, polymorph or solvate of a compound” intends the inclusive meaning of “and/or”, in that materials meeting more than one of the stated criteria are included, e.g., a material that is both a salt and a solvate is encompassed.

The compounds of the present disclosure can contain unnatural proportions of atomic isotopes at one or more of the atoms that constitute such compounds. For example, the compounds can be radiolabeled with radioactive isotopes, such as for example tritium (^{3H}), iodine-125 (¹²⁵I) or carbon-14 (^{14C}). All isotopic variations of the compounds of the present disclosure, whether radioactive or not, are intended to be encompassed within the scope of the present disclosure. Compounds described herein, in which one or more of the hydrogen atoms are replaced with another stable isotope of hydrogen (i.e., deuterium) or a radioactive isotope (i.e., tritium), are part of this disclosure.

### Compositions Including Stereoisomers

Compounds of the present disclosure can exist in particular geometric or stereoisomeric forms. The present disclosure contemplates all such compounds, including cis- and trans-isomers, (−)- and (+)-enantiomers, diastereomers, (D)-isomers, (L)-isomers, the racemic mixtures thereof, and other mixtures thereof, such as enantiomerically or diastereomerically enriched mixtures, as falling within the scope of the present disclosure. Additional asymmetric carbon atoms can be present in a substituent such as an alkyl group. All such isomers, as well as mixtures thereof, are intended to be included in this disclosure. When the compounds described herein contain olefinic double bonds or other centers of geometric asymmetry, and unless specified otherwise, it is intended that the compounds include both E and Z geometric isomers. Likewise, all tautomeric forms and mixtures of tautomers are included.

Optically active (R)- and (S)-isomers and d and 1 isomers can be prepared using chiral synthons or chiral reagents, or resolved using conventional techniques. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound. If, for instance, a particular enantiomer of a compound of the present disclosure is desired, it can be prepared by asymmetric synthesis, or by derivatization with a chiral auxiliary, where the resulting diastereomeric mixture is separated and the auxiliary group cleaved to provide the pure desired enantiomers. Alternatively, where the molecule contains a basic functional group, such as an amino group, or an acidic functional group, such as a carboxyl group, diastereomeric salts can be formed with an appropriate optically active acid or base, followed by resolution of the diastereomers thus formed by fractional crystallization or chromatographic means known in the art, and subsequent recovery of the pure enantiomers. In addition, separation of enantiomers and diastereomers is frequently accomplished using chromatography employing chiral, stationary phases, optionally in combination with chemical derivatization (e.g., formation of carbamates from amines).

As used herein, the term “chiral”, “enantiomerically enriched” or “diastereomerically enriched” refers to a compound having an enantiomeric excess (ee) or a diastereomeric excess (de) of greater than about 50%, for example, greater than about 70%, such as greater than about 90%. In one embodiment, the compositions have higher than about 90% enantiomeric or diastereomeric excess, e.g., those compositions with greater than about 95%, greater than about 97% and greater than about 99% ee or de.

The terms “enantiomeric excess” and “diastereomeric excess” are used in their conventional sense. Compounds with a single stereocenter are referred to as being present in “enantiomeric excess”, those with at least two stereocenters are referred to as being present in “diastereomeric excess”. The value of ee will be a number from 0 to 100, zero being racemic and 100 being enantiomerically pure. For example, a 90% ee reflects the presence of 95% of one enantiomer and 5% of the other(s) in the material in question.

Hence, in one embodiment, the disclosure provides methods using a composition including a first stereoisomer and at least one additional stereoisomer of a compound of the present disclosure. The first stereoisomer can be present in a diastereomeric or enantiomeric excess of at least about 80%, such as at least about 90%, and for example, at least about 95%. In another embodiment, the first stereoisomer is present in a diastereomeric or enantiomeric excess of at least about 96%, at least about 97%, at least about 98%, at least about 99% or at least about 99.5%. In yet another embodiment, the compounds of the present disclosure is enantiomerically or diastereomerically pure (diastereomeric or enantiomeric excess is about 100%). Enantiomeric or diastereomeric excess can be determined relative to exactly one other stereoisomer, or can be determined relative to the sum of at least two other stereoisomers. In an exemplary embodiment, enantiomeric or diastereomeric excess is determined relative to all other detectable stereoisomers, which are present in the mixture. Stereoisomers are detectable if a concentration of such stereoisomer in the analyzed mixture can be determined using common analytical methods, such as chiral HPLC.

The term “JNK-mediated fibrotic condition”, “c-Jun N-terminal kinase mediated disorder” or any other variation thereof, as used herein means any disease or other condition in which JNK is known to play a role in the initiation, progression, or maintenance of fibrosis in an individual.

“Fibrosis” is the formation of fibrous connective tissue in an organ or tissue in a reparative or reactive process, or any abnormal proliferation and/or differentiation of fibroblasts that results in a pathological formation of such fibrous connective tissue.

The term “disease, disorder or condition associated with fibrosis” as used herein means any disease or other condition in which JNK is known to play a role in the initiation, progression, or maintenance of fibrosis in an individual. Exemplary resulting diseases, disorders or conditions include, without limitation, idiopathic pulmonary fibrosis, organ fibrosis, interstitial lung disease, skin fibrosis, diabetic nephropathy, liver fibrosis, liver cirrhosis, nonalcoholic steatohepatitis (NASH), rheumatoid arthritis, fibrosarcomas, keloids and hypertrophic scars, arteriosclerosis, kidney disease, macular degeneration, retinal and vitreal retinopathy, surgical complications, chemotherapeutic drug-induced fibrosis, radiation-induced fibrosis, accidental injury, burns, local scleroderma, and systemic scleroderma. Preferably, the fibrotic disease is idiopathic pulmonary fibrosis, NASH, or cardiac fibrosis.

### Compositions

The present disclosure provides a method of treating diseases, conditions or disorders using inhibitors of the Jun N-terminal kinase pathway. In various aspects, the present disclosure provides for treatment of diseases, disorders and conditions associated with fibrosis in a mammal using a compound having a structure according to Formula (I):

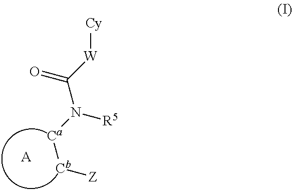

or a salt or solvate thereof, wherein ring A is 5-membered heteroaryl comprising a sulfur atom, wherein the heteroaryl is optionally substituted with 1 or 2 substituents independently chosen from alkyl, alkenyl, alkynyl, haloalkyl, heteroalkyl, C₃-C₁₀-cycloalkyl, 3- to 8-membered heterocycloalkyl, aryl, 5- or 6-membered heteroaryl, CN, halogen, OR¹², SR¹², NR¹²R¹³, C(O)R¹⁴, C(O)NR¹²R¹³, OC(O)NR¹²R¹³, C(O)OR¹², NR¹⁵C(O)R¹⁴, NR¹⁵C(O)OR¹², NR¹⁵C(O)NR¹²R¹³, NR¹⁵C(S)NR¹²R¹³, NR¹⁵S(O)₂R¹⁴, S(O)₂NR¹²R¹³, S(O)R¹⁴ and S(O)₂R¹⁴, wherein R¹², R¹³ and R¹⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹² and R¹³, together with the nitrogen atom to which they are bound form a 5- to 7-membered heterocyclic ring; and R¹⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; C^{a} and C^{b} are carbon atoms, which are adjacent to each other and are part of ring A; Z is 5- or 6-membered heteroaryl, with the proviso that (i) when ring A is thiophene, then Z is not a heteroaryl chosen from benzoimidazole, thiazole, and benzothiazole; (ii) when ring A is thiazole, then Z is not benzoimidazole; (iii) when ring A is thiophene, then Z is not substituted oxadiazole; and (iv) when ring A is thiophene, then Z is not pyrimidinone; R⁵ is chosen from H, acyl, substituted or unsubstituted C₁-C₆ alkyl, and C₃-C₆ cycloalkyl; W is chosen from C₁-C₄ alkylene, wherein the alkylene is optionally substituted with 1-4 substituents independently chosen from alkyl, alkenyl, alkynyl, haloalkyl, heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R⁴⁴, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R, and S(O)₂R⁴⁴, wherein R⁴², R⁴³ and R⁴⁵ are members independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁴² and R⁴³, together with the nitrogen atom to which they are bound are optionally joined to form a 5- to 7-membered heterocyclic ring; and R⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; Cy is chosen from cycloalkyl, heterocycloalkyl, aryl, and heteroaryl, wherein the cycloalkyl, heterocycloalkyl, aryl or heteroaryl is optionally substituted with 1-6 substituents independently chosen from substituted or unsubstituted alkyl, substituted or unsubstituted alkenyl, substituted or unsubstituted alkynyl, haloalkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, CN, halogen, OR⁵², SR⁵², ═O, NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl.

The present disclosure further provides for a pharmaceutical composition for the treatment or prevention of a disease, disorder or condition associated with fibrosis comprising a compound according to Formula (I) and a pharmaceutically acceptable carrier.

In specific aspects, the present disclosure provides for treatment of diseases, disorders and conditions associated with fibrosis in a mammal using a compound having a structure according to Formula (I):

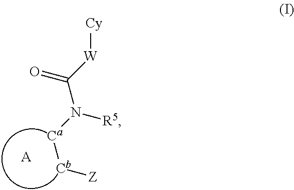

or a salt or solvate thereof, wherein ring A is 5-membered heteroaryl comprising a sulfur atom, wherein the heteroaryl is optionally substituted with 1 or 2 substituents independently chosen from alkyl, alkenyl, alkynyl, haloalkyl, heteroalkyl, C₃-C₁₀-cycloalkyl, 3- to 8-membered heterocycloalkyl, aryl, 5- or 6-membered heteroaryl, CN, halogen, OR¹², SR¹², NR¹²R¹³, C(O)R¹⁴, C(O)NR¹²R¹³, OC(O)NR¹²R¹³, C(O)OR¹², NR¹⁵C(O)R¹⁴, NR¹⁵C(O)OR¹², NR¹⁵C(O)NR¹²R¹³, NR¹⁵C(S)NR¹²R¹³, NR¹⁵S(O)₂R¹⁴, S(O)₂NR¹²R¹³, S(O)R¹⁴ and S(O)₂R¹⁴, wherein R¹², R¹³ and R¹⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹² and R¹³, together with the nitrogen atom to which they are bound form a 5- to 7-membered heterocyclic ring; and R¹⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; C^{a} and C^{b} are carbon atoms, which are adjacent to each other and are part of ring A; Z is triazole optionally substituted with alkyl, cycloalkyl, alkenyl, alkynyl, heteroalkyl, heterocycloalkyl, aryl, heteroaryl, —R^{a}, —OR^{a}, —SR^{a}, ═O, ═NR^{a}, ═N—OR^{a}, —NR^{a}R^{b}, -halogen, —SiR^{a}R^{b}R^{c}, —OC(O)R^{a}, —C(O)R^{e}, —C(O)OR^{a}, —C(O)NR^{a}R^{b}, —OC(O)NR^{a}R^{b}, —NR^{c}C(O)R^{e}, —NR^{c}C(O)NR^{a}R^{b}, —NR^{c}C(S)NR^{a}R^{b}, —NR^{c}C(O)OR^{a}, —NR^{c}C(NR^{a}R^{b})═NR^{d}, S(O)R^{e}, —S(O)₂R^{e}, —S(O)₂NR^{a}R^{b}, —NR^{c}S(O)₂R^{a}, —CN, —NO₂, —N₃, —CH(Ph)₂, fluoro(C₁-C₄)alkoxy, and fluoro(C₁-C₄)alkyl, wherein R^{a}, R^{b}, R^{c}, R^{d} and R^{e} each independently hydrogen, C₁-C₂₄ alkyl, C₃-C₁₀ cycloalkyl, C₁-C₂₄ heteroalkyl, C₃-C₁₀ heterocycloalkyl, aryl, heteroaryl, arylalkyl and heteroarylalkyl; and wherein where two R groups are attached to the same nitrogen atom, they can be combined with the nitrogen atom to form a 5-, 6-, or 7-membered ring; R⁵ is chosen from H, acyl, substituted or unsubstituted C₁-C₆ alkyl, and C₃-C₆ cycloalkyl; W is chosen from C₁-C₄ alkylene, wherein the alkylene is optionally substituted with 1-4 substituents independently chosen from alkyl, alkenyl, alkynyl, haloalkyl, heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R⁴⁴, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R, and S(O)₂R⁴⁴, wherein R⁴², R⁴³, and R⁴⁵ are members independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁴² and R⁴³, together with the nitrogen atom to which they are bound are optionally joined to form a 5- to 7-membered heterocyclic ring; and R⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; Cy is chosen from cycloalkyl, heterocycloalkyl, aryl, and heteroaryl, wherein the cycloalkyl, heterocycloalkyl, aryl or heteroaryl is optionally substituted with 1-6 substituents independently chosen from substituted or unsubstituted alkyl, substituted or unsubstituted alkenyl, substituted or unsubstituted alkynyl, haloalkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, CN, halogen, OR⁵², SR⁵², ═O, NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; wherein the aryl groups are chosen from 5-, 6- or 7-membered, aromatic carbocyclic group having a single ring or being fused to other aromatic or non-aromatic rings; and wherein the heteroaryl groups are chosen from polyunsaturated, 5-, 6- or 7-membered aromatic moiety containing at least one heteroatom chosen from N, O, S, Si and B, wherein the nitrogen and sulphur atoms are optionally oxidized, and the nitrogen atom(s) are optionally quarternized, and wherein the heteroaryl groups can be a single ring or be fused to other aryl, heteroaryl, cycloalkyl or heterocycloalkyl rings.

In certain specific aspects of the invention, the present disclosure provides methods for treatment of diseases, disorders and conditions associated with fibrosis in a mammal using a compound having a structure according to Formula (IV), Formula (V), Formula (VI) or Formula (VII):

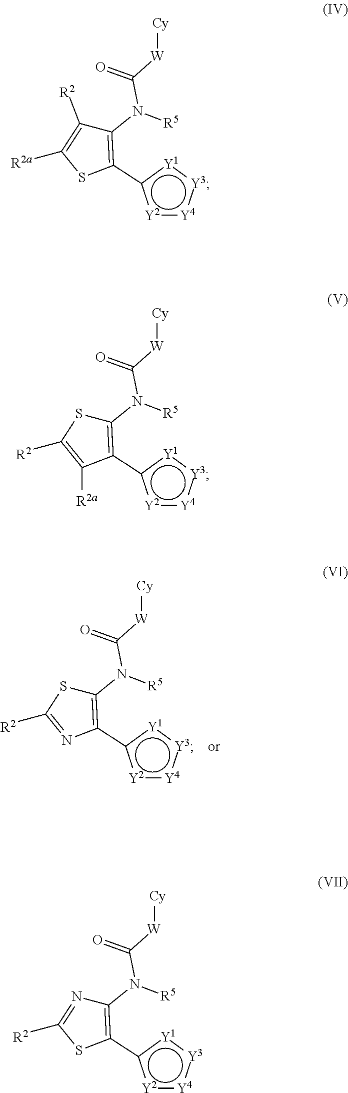

or a salt or solvate thereof, wherein R2 and R2a are independently chosen from H, C1-C4-alkyl, C1-C4-alkenyl, C1-C4-alkynyl, C1-C4-haloalkyl, 2- to 4-membered heteroalkyl, C3-C6-cycloalkyl, 3- to 6-membered heterocycloalkyl, CN, and halogen; wherein the moiety

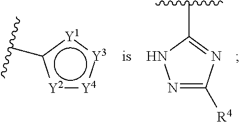

wherein R4 is chosen from H, C1-C4 alkyl, C1-C4 alkenyl, C1-C4 alkynyl, C1-C4 haloalkyl, C3-C6 cycloalkyl, 3- to 6-membered heterocycloalkyl, aryl, 5- or 6-membered heteroaryl, CN, halogen, OR17, SR17 and NR17R18, wherein R17 and R18 are independently chosen from H, acyl, C1-C6-alkyl, 2- to 6-membered heteroalkyl, aryl, 5- or 6-membered heteroaryl, C3-C8 cycloalkyl and 3- to 8-membered heterocycloalkyl, wherein R17 and R18, together with the nitrogen atom to which they are bound are optionally joined to form a 5- to 7-membered heterocyclic ring.

In other specific aspects of the invention, the present disclosure provides methods for treatment of diseases, disorders and conditions associated with fibrosis in a mammal using a compound having a structure according to according to Formula (IV), Formula (V), Formula (VI) or Formula (VII):

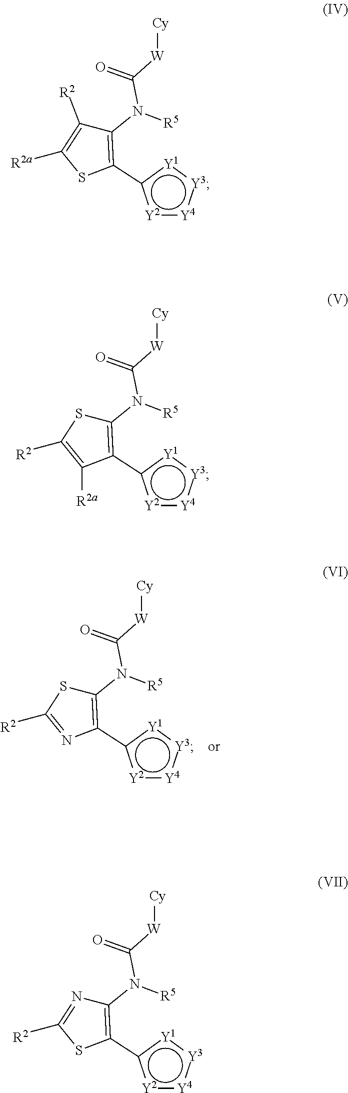

or a salt or solvate thereof; wherein ring A is chosen from thiophene and thiazole, wherein the thiophene or the thiazole is optionally substituted with 1 or 2 substituents chosen from C₁-C₄-alkyl, C₁-C₄-alkenyl, C₁-C₄-alkynyl, C₁-C₄-haloalkyl, 2- to 4-membered heteroalkyl, C₃-C₆-cycloalkyl, 3- to 6-membered heterocycloalkyl, CN, and halogen.

In other specific aspects of the invention, the present disclosure provides treatment of diseases, disorders and conditions associated with fibrosis in a mammal using a compound having a structure according to Formula (IVa), Formula (Va), Formula (VIa) or Formula (VIIa):

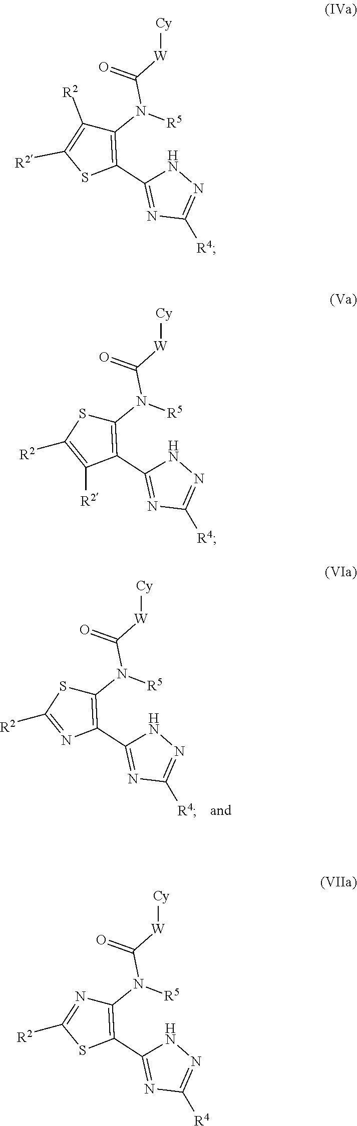

or a salt or solvate thereof, wherein R⁴ is chosen from H, methyl, and cyclopropyl.

In certain specific aspects of the invention W of the composition used in the methods of the invention is methylene (—CH₂—). In other specific aspects of the invention, R⁵ of the composition used in the methods of the invention is H. In yet other specific aspects of the invention, Cy of the composition used in the methods of the invention is chosen from phenyl, naphthyl, quinoline, isoquinoline, quinoxaline, quinazoline, quinolin-2-one, 3,4-dihydroquinolin-2-one, 3,4-dihydro-1,5-naphthyridin-2-one, and 3,4-dihydro-1,6-naphthyridin-2-one, each optionally substituted with 1-6 substituents independently chosen from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR⁵², SR⁵², NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ or S(O)₂R⁵⁴

In some embodiments, Cy of the composition used in the methods of the invention is optionally substituted quinolone, substituted isoquinoline, substituted quinolin-2-one, quinolin-2-one substituted with at least one C₁-C₆-haloalkyl, substituted 3,4-dihydro-1,6-naphthyridin-2-one, substituted 3,4-dihydro-1,5-naphthyridin-2-one, or unsubstituted 3,4-dihydro-1,5-naphthyridin-2-one.

In other embodiments, Cy of the composition used in the methods of the invention is selected from the group consisting of:

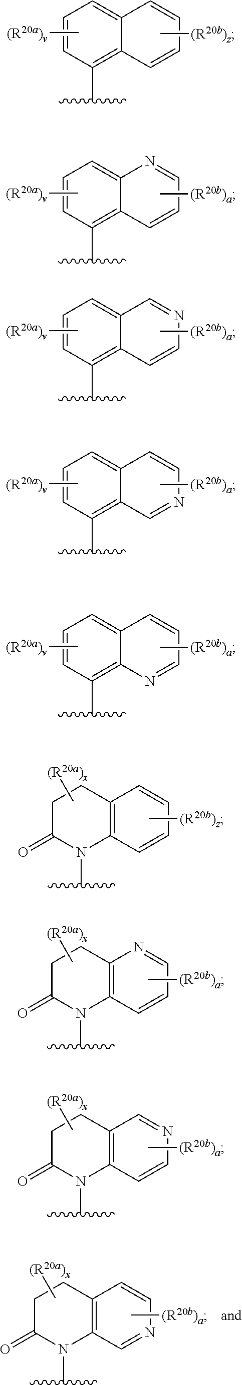

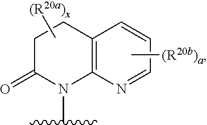

wherein: R^{20a} and R^{20b} are each independently chosen from C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR⁵², SR⁵², NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴; v is an integer from 0 to 3; x is an integer from 0 to 4; z is an integer from 0 to 4; and a is an integer from 0 to 3.

In a specific embodiment, the composition used in the methods of the invention is N-(4-bromo-3-(1H-1,2,4-triazol-5-yl)thiophen-2-yl)-2-(2-oxo-6-(trifluoromethyl)quinolin-1(2H)-yl)acetamide.

In another specific embodiment, the composition used in the methods of the invention is N-(4-bromo-3-(1H-1,2,4-triazol-5-yl)thiophen-2-yl)-2-(2-oxo-3,4-dihydro-1,5-naphthyridin-1(2H)-yl)acetamide.

The present disclosure also provides for treatment of diseases, disorders and conditions associated with peripheral fibrosis using a compound having a structure according to Formula (VIII):

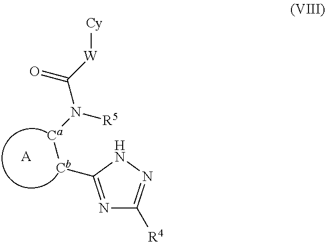

or a tautomer, mixture of tautomers, salt or solvate thereof, wherein ring A is 5- or 6-membered heteroaryl, wherein the heteroaryl is optionally substituted with 1-3 substituents independently chosen from alkyl, alkenyl, alkynyl, haloalkyl, heteroalkyl, C₃-C₁₀-cycloalkyl, 3- to 8-membered heterocycloalkyl, aryl, 5- or 6-membered heteroaryl, CN, halogen, OR¹², SR¹², NR¹²R¹³, C(O)R¹⁴, C(O)NR¹²R¹³, OC(O)NR¹²R¹³, C(O)OR¹², NR¹⁵C(O)R¹⁴, NR¹⁵C(O)OR¹², NR¹⁵C(O)NR¹²R¹³, NR¹⁵C(S)NR¹²R¹³, NR¹⁵S(O)₂R¹⁴, S(O)₂NR¹²R¹³, S(O)R¹⁴ and S(O)₂R¹⁴, wherein R¹², R¹³ and R¹⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹² and R¹³, together with the nitrogen atom to which they are bound form a 5- to 7-membered heterocyclic ring; and R¹⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; C^{a} and C^{b} are carbon atoms, which are adjacent to each other and, which are part of ring A; R⁴ is chosen from H, independently chosen from H, C₁-C₄ alkyl, C₁-C₄ alkenyl, C₁-C₄ alkynyl, C₁-C₄ haloalkyl, C₃-C₆ cycloalkyl, 3- to 6-membered heterocycloalkyl, aryl, and 5- or 6-membered heteroaryl, CN, halogen, OR¹⁷, SR¹⁷ and NR¹⁷R¹⁸, wherein R¹⁷ and R¹⁸ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹⁷ and R¹⁸, together with the nitrogen atom to which they are bound form a 5- to 7-membered heterocyclic ring; R⁵ is chosen from H, acyl, C₁-C₆ alkyl, and C₃-C₆ cycloalkyl; W is chosen from C₁-C₄ alkylene, wherein the alkylene is optionally substituted with from 1 to 4 substituents chosen from alkyl, alkenyl, alkynyl, haloalkyl, heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R⁴⁴, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R⁴⁴, and S(O)₂R⁴⁴, wherein R⁴², R⁴³ and R⁴⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁴² and R⁴³, together with the nitrogen atom to which they are bound are optionally joined to form a 5- to 7-membered heterocyclic ring; and R⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; Cy is chosen from cycloalkyl, heterocycloalkyl, aryl, and heteroaryl, wherein the cycloalkyl, heterocycloalkyl, aryl or heteroaryl is optionally substituted with 1-6 substituents independently chosen from substituted or unsubstituted alkyl, substituted or unsubstituted alkenyl, substituted or unsubstituted alkynyl, haloalkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, CN, halogen, OR⁵², SR⁵², NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl.

In some embodiments, the composition for use in the methods of the present disclosure have a structure according to Formula VIII where ring A is chosen from thiophene, thiazole and pyrazole, wherein the thiophene, the thiazole or the pyrazole is optionally substituted with 1 or 2 substituents chosen from C₁-C₄-alkyl, C₁-C₄-alkenyl, C₁-C₄-alkynyl, C₁-C₄-haloalkyl, 2- to 4-membered heteroalkyl, C₃-C₆-cycloalkyl, 3- to 6-membered heterocycloalkyl, CN, and halogen. In other specific embodiments, the compounds for use in the methods of the present disclosure have a structure according to Formula (IVa), Formula (Va), Formula (VIa) or Formula (VIIa):

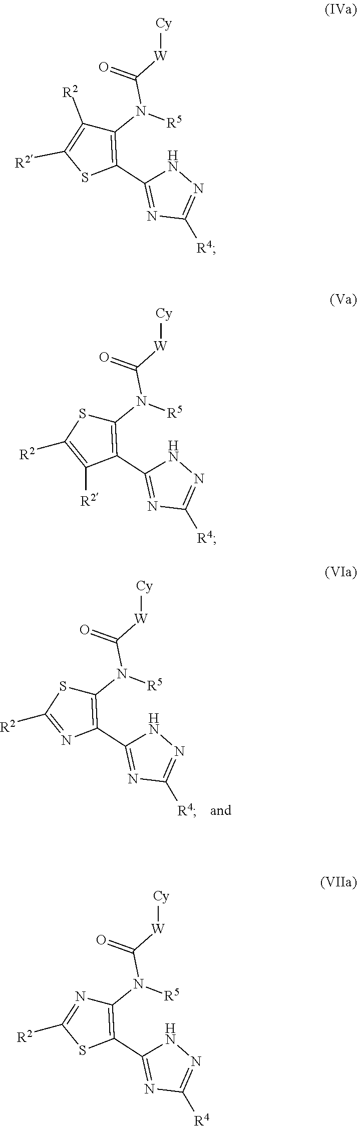

or a salt or solvate thereof, wherein R² and R²′ are independently chosen from H, C₁-C₄-alkyl, C₁-C₄-alkenyl, C₁-C₄-alkynyl, C₁-C₄-haloalkyl, 2- to 4-membered heteroalkyl, C₃-C₆-cycloalkyl, 3- to 6-membered heterocycloalkyl, CN, and halogen.

In certain specific embodiments, the compounds of Formula (IVa), Formula (Va), Formula (VIII), Formula (VIa) or Formula (VIIa) contain a R⁴ is chosen from H, methyl and cyclopropyl. In other specific embodiments, the compound of Formula (VIII) has a W which is methylene (—CH₂—).

In certain specific embodiments, the compounds for use in the present disclosure have a structure according to Formula (X) or Formula (XI):

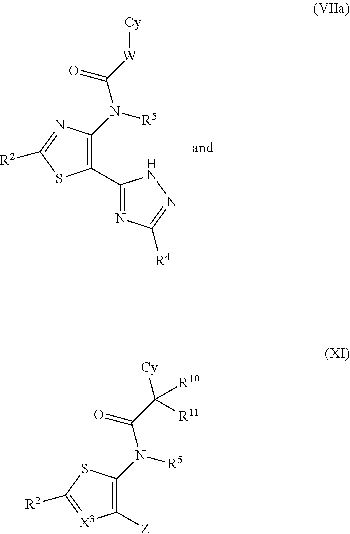

Wherein X¹ is chosen from N and CR^{2a}; R² and R^{2a} are independently chosen from H, C₁-C₄-alkyl, C₁-C₄-alkenyl, C₁-C₄-alkynyl, C₁-C₄-haloalkyl, 2- to 4-membered heteroalkyl, C₃-C₆-cycloalkyl, 3- to 6-membered heterocycloalkyl, CN, and halogen; R¹⁰ and R¹¹ are independently chosen from H, C₁-C₆-alkyl, C₁-C₆-alkenyl, C₁-C₆-alkynyl, C₁-C₆-haloalkyl, 2- to 6-membered heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R⁴⁴ and S(O)₂R⁴⁴, wherein R⁴², R⁴³ and R⁴⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R⁴² and R⁴³, together with the nitrogen atom to which they are bound form a 5- to 7-membered heterocyclic ring; and R⁴⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; R⁵ is chosen from H and substituted or unsubstituted C₁-C₆ alkyl Cy is chosen from cycloalkyl, heterocycloalkyl, aryl, and heteroaryl, wherein the cycloalkyl, heterocycloalkyl, aryl or heteroaryl is optionally substituted with from 1 to 6 substituents independently chosen from C₁-C₆-alkyl, C₁-C₆-alkenyl, C₁-C₆-alkynyl, C₁-C₆-haloalkyl, 2- to 6-membered heteroalkyl, C₃-C₁₂-cycloalkyl, 3- to 8-membered heterocycloalkyl, aryl, 5- or 6-membered heteroaryl, CN, halogen, OR⁵², SR⁵², NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; and Z is chosen from:

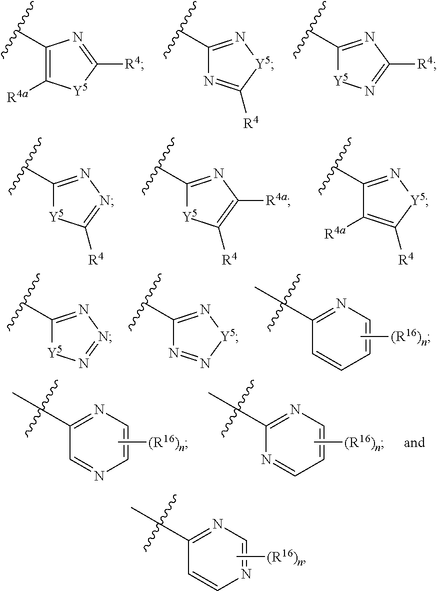

wherein Y⁵ is chosen from O, S and NR³, wherein R³ is chosen from H, alkyl, alkenyl, alkynyl, haloalkyl, cycloalkyl, 3- to 8-membered heterocycloalkyl, aryl, and 5- or 6-membered heteroaryl; and R⁴, R^{4a} and R¹⁶ are independently chosen from H, alkyl, alkenyl, alkynyl, haloalkyl, cycloalkyl, 3- to 8-membered heterocycloalkyl, aryl, 5- or 6-membered heteroaryl, CN, halogen, OR¹⁷, SR¹⁷ and NR¹⁷R¹⁸, wherein R¹⁷ and R¹⁸ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹⁷ and R¹⁸, together with the nitrogen atom to which they are bound form a 5- to 7-membered heterocyclic ring, or two of R⁴, R^{4a} and R³, together with the atoms to which they are attached, form a 5- to 7-membered ring, or adjacent R¹⁶ groups, together with the carbon atoms to which they are attached, form a 5- to 7-membered ring; n is an integer chosen from 0 to 4; and m is an integer chosen from 0 to 3.

In certain embodiments, the compound according to Formula (XI) for use in the present disclosure comprises an R¹⁰ and R¹¹ that are both H. In other certain embodiments, the compound according to Formula (XI) for use in the present disclosure comprises R² and R^{2a} bindependently chosen from H, methyl, CF₃, CN, F, Cl and Br.

In other certain embodiments, the compound according to Formula (I), (VIII) or (XI) for use in the present disclosure comprises an R⁵ that is H.

In certain aspects, the compound according to Formula (I), (VIII) or (XI) for use in the methods of the present disclosure comprises Cy chosen from phenyl, naphthyl, quinoline, isoquinoline, quinoxaline, quinazoline, 3,4-dihydroquinolin-2-one, and 3,4-dihydro-1,6-naphthyridin-2-one, each optionally substituted with 1-6 substituents independently chosen from C₁-C₆-alkyl, C₁-C₆-alkenyl, C₁-C₆-alkynyl, C₁-C₆-haloalkyl, 2- to 6-membered heteroalkyl, C₃-C₁₂-cycloalkyl, 3- to 8-membered heterocycloalkyl, aryl, 5- or 6-membered heteroaryl, CN, halogen, OR⁵², SR⁵², NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl.

In one specific embodiment, the compound used for the treatment of diseases, disorders and conditions associated with fibrosis in a mammal using IGP001, N-(4-bromo-3-(1H-1,2,4-triazol-5-yl)thiophen-2-yl)-2-(2-oxo-6-(trifluoromethyl)quinolin-1(2H)-yl)acetamide, a compound having a structure according to Formula (XXI):

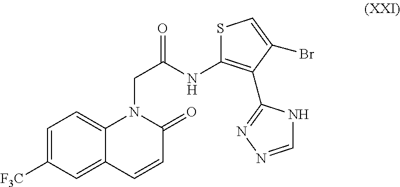 [SMILES: O=C(Cn1c(=O)ccc2cc(C(F)(F)F)ccc21)Nc1scc(Br)c1-c1nnc[nH]1]

In another specific embodiment, the compound used for the treatment of diseases, disorders and conditions associated with fibrosis in a mammal using IGP002, N-(4-bromo-3-(1H-1,2,4-triazol-5-yl)thiophen-2-yl)-2-(2-oxo-1,5-naphthyridin-1(2H)-yl)acetamide, a compound having a structure according to Formula (XXII):

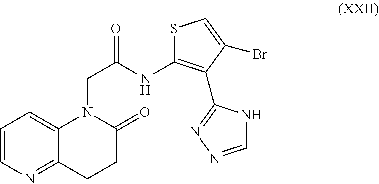 [SMILES: O=C(CN1C(=O)CCc2ncccc21)Nc1scc(Br)c1-c1nnc[nH]1]

In other specific embodiments, the present disclosure comprises use of the following compounds in the methods of the invention are chosen from:
N-(2-(1H-1,2,4-triazol-5-yl)thiophen-3-yl)-2-(naphthalen-1-yl)acetamide;
N-(2-(3-methyl-1H-1,2,4-triazol-5-yl)thiophen-3-yl)-2-(naphthalen-1-yl)acetamide;
N-(2-(1,3-dimethyl-1H-1,2,4-triazol-5-yl)thiophen-3-yl)-2-(naphthalen-1-yl)acetamide;
N-(2-(1-methyl-1H-1,2,4-triazol-5-yl)thiophen-3-yl)-2-(naphthalen-1-yl)acetamide;
2-(4-methoxyphenyl)-N-(2-(3-methyl-1H-1,2,4-triazol-5-yl)thiophen-3-yl)acetamide;
N-(2-(1H-1,2,4-triazol-5-yl)thiophen-3-yl)-2-(4-methoxyphenyl)acetamide;
N-(2-(1H-1,2,4-triazol-1-yl)thiophen-3-yl)-2-(4-methoxyphenyl)acetamide;
2-(4-methoxyphenyl)-N-(4-methyl-3-(3-methyl-1H-1,2,4-triazol-5-yl)thiophen-2-yl)acetamide;
N-(2-(2H-1,2,3-triazol-2-yl)thiophen-3-yl)-2-(4-methoxyphenyl)acetamide;
N-(2-(3-cyclopropyl-1H-1,2,4-triazol-5-yl)thiophen-3-yl)-2-(4-methoxyphenyl)acetamide;
N-(2-(3-ethyl-1H-1,2,4-triazol-5-yl)thiophen-3-yl)-2-(4-methoxyphenyl)acetamide;
N-(2-(3-tert-butyl-1H-1,2,4-triazol-5-yl)thiophen-3-yl)-2-(4-methoxyphenyl)acetamide;
2-(4-methoxyphenyl)-N-(2-(3-(tetrahydrofuran-2-yl)-1H-1,2,4-triazol-5-yl)thiophen-3-yl)acetamide;
2-(4-methoxyphenyl)-N-(2-(3-(trifluoromethyl)-1H-1,2,4-triazol-5-yl)thiophen-3-yl)acetamide;
N-(4-methyl-3-(1H-1,2,4-triazol-5-yl)thiophen-2-yl)-2-(2-oxo-3,4-dihydroquinolin-1(2H)-yl)acetamide;
N-(4-methyl-3-(3-methyl-1H-1,2,4-triazol-5-yl)thiophen-2-yl)-2-(2-oxo-3,4-dihydroquinolin-1(2H)-yl)acetamide;
2-(4-methoxyphenyl)-N-(2-(3-(pyridin-4-yl)-1H-1,2,4-triazol-5-yl)thiophen-3-yl)acetamide;
N-(2-(3-amino-1H-1,2,4-triazol-5-yl)thiophen-3-yl)-2-(4-methoxyphenyl)acetamide;
N-(4-chloro-3-(1H-1,2,4-triazol-5-yl)thiophen-2-yl)-2-(2-oxo-3,4-dihydroquinolin-1(2H)-yl)acetamide;
N-(3-(1H-1,2,4-triazol-5-yl)thiophen-2-yl)-2-(2-oxo-3,4-dihydroquinolin-1(2H)-yl)acetamide;
N-(4-chloro-3-(1H-1,2,4-triazol-5-yl)thiophen-2-yl)-2-(isoquinolin-5-yl)acetamide;
N-(4-chloro-3-(1H-1,2,4-triazol-5-yl)thiophen-2-yl)-2-(quinolin-5-yl)acetamide;
N-(2-(1H-1,2,4-triazol-5-yl)thiophen-3-yl)-2-(2,3-dihydrobenzo[b][1,4]dioxin-6-yl)acetamide;
N-(4-methyl-3-(1H-1,2,4-triazol-5-yl)thiophen-2-yl)-2-(quinolin-5-yl)acetamide;
2-(2,3-dihydrobenzo[b][1,4]dioxin-6-yl)-N-(2-(3-methyl-1H-1,2,4-triazol-5-yl)thiophen-3-yl)acetamide;
N-(2-(1H-1,2,4-triazol-5-yl)thiophen-3-yl)-2-(quinolin-5-yl)acetamide;
N-(4-methyl-3-(3-methyl-1H-1,2,4-triazol-5-yl)thiophen-2-yl)-2-(quinolin-5-yl)acetamide;
N-(4-methyl-3-(1H-1,2,4-triazol-5-yl)thiophen-2-yl)-2-(quinoxalin-5-yl)acetamide;
N-(4-methyl-3-(3-methyl-1H-1,2,4-triazol-5-yl)thiophen-2-yl)-2-(quinoxalin-5-yl)acetamide;
N-(4-methyl-3-(1H-1,2,4-triazol-5-yl)thiophen-2-yl)-2-(4-(3-(piperidin-1-yl)propoxy)phenyl)acetamide;
N-(4-methyl-3-(3-methyl-1H-1,2,4-triazol-5-yl)thiophen-2-yl)-2-(4-(3-(piperidin-1-yl)propoxy)phenyl)acetamide;
2-(4-(2-(1H-imidazol-1-yl)ethoxy)phenyl)-N-(4-methyl-3-(1H-1,2,4-triazol-5-yl)thiophen-2-yl)acetamide;
N-(4-bromo-3-(1H-1,2,4-triazol-5-yl)thiophen-2-yl)-2-(isoquinolin-5-yl)acetamide;
N-(4-bromo-3-(1H-1,2,4-triazol-5-yl)thiophen-2-yl)-2-(quinolin-5-yl)acetamide;
N-(4-bromo-3-(1H-1,2,4-triazol-5-yl)thiophen-2-yl)-2-(2-oxo-3,4-dihydroquinolin-1(2H)-yl)acetamide;
N-(4-cyano-3-(1H-1,2,4-triazol-5-yl)thiophen-2-yl)-2-(isoquinolin-5-yl)acetamide;
N-(4-cyano-3-(1H-1,2,4-triazol-5-yl)thiophen-2-yl)-2-(2-oxo-3,4-dihydroquinolin-1(2H)-yl)acetamide;
N-(2-(3-methyl-H-1,2,4-triazol-5-yl)thiophen-3-yl)-2-(4-(2-oxopyrrolidin-1-yl)phenyl)acetamide;
N-(4-methyl-3-(5-methyl-4H-1,2,4-triazol-3-yl)thiophen-2-yl)-2-(4-(pyridin-4-yl)phenyl)acetamide;
N-(4-cyano-3-(4H-1,2,4-triazol-3-yl)thiophen-2-yl)-2-(quinolin-5-yl)acetamide;
N-(4-bromo-3-(1H-1,2,4-triazol-5-yl)thiophen-2-yl)-2-(2-oxo-7-(trifluoromethyl)quinolin-1(2H)-yl)acetamide;
N-(4-bromo-3-(1H-1,2,4-triazol-5-yl)thiophen-2-yl)-2-(6-fluoro-2-oxo-3,4-dihydroquinolin-1(2H)-yl)acetamide;
N-(4-bromo-3-(1H-1,2,4-triazol-5-yl)thiophen-2-yl)-2-(7-fluoro-2-oxoquinolin-1(2H)-yl)acetamide;
N-(4-bromo-3-(1H-1,2,4-triazol-5-yl)thiophen-2-yl)-2-(7-chloro-2-oxo-3,4-dihydroquinolin-1(2H)-yl)acetamide;
N-(4-bromo-3-(1H-1,2,4-triazol-5-yl)thiophen-2-yl)-2-(6,7-difluoro-2-oxoquinolin-1(2H)-yl)acetamide;
N-(4-bromo-3-(1H-1,2,4-triazol-5-yl)thiophen-2-yl)-2-(2-oxo-6-(trifluoromethyl)quinolin-1(2H)-yl)acetamide;
N-(4-bromo-3-(1H-1,2,4-triazol-5-yl)thiophen-2-yl)-2-(6-fluoro-2-oxoquinolin-1(2H)-yl)acetamide;
2-(isoquinolin-5-yl)-N-(2-(4-methylthiazol-2-yl)thiophen-3-yl)acetamide; 2-(isoquinolin-5-yl)-N-(2-(thiazol-4-yl)thiophen-3-yl)acetamide;
2-(2-oxo-3,4-dihydro-1,6-naphthyridin-1(2H)-yl)-N-(2-(thiazol-4-yl)thiophen-3-yl)acetamide;
2-(2-oxo-3,4-dihydroquinolin-1(2H)-yl)-N-(2-(thiazol-4-yl)thiophen-3-yl)acetamide;
2-(isoquinolin-5-yl)-N-(2-(2-methoxythiazol-4-yl)thiophen-3-yl)acetamide;
N-(2-(2-chlorothiazol-4-yl)thiophen-3-yl)-2-(isoquinolin-5-yl)acetamide;
2-(isoquinolin-5-yl)-N-(2-(thiazol-2-yl)thiophen-3-yl)acetamide;
2-(isoquinolin-5-yl)-N-(2-(5-methylthiazol-2-yl)thiophen-3-yl)acetamide;
2-(4-(3-(piperidin-1-yl)propoxy)phenyl)-N-(2-(thiazol-4-yl)thiophen-3-yl)acetamide;
N-(3-(benzo[d]thiazol-2-yl)-4-methylthiophen-2-yl)-2-(isoquinolin-5-yl)acetamide;
2-(4-methoxyphenyl)-N-(2-(oxazol-2-yl)thiophen-3-yl)acetamide;
2-(isoquinolin-5-yl)-N-(2-(oxazol-2-yl)thiophen-3-yl)acetamide;
2-(4-methoxyphenyl)-N-(3-(5-methyl-1,2,4-oxadiazol-3-yl)thiophen-2-yl)acetamide;
N-(2-(1,3,4-oxadiazol-2-yl)thiophen-3-yl)-2-(naphthalen-1-yl)acetamide;
2-(4-methoxyphenyl)-N-(2-(5-methyl-1,3,4-oxadiazol-2-yl)thiophen-3-yl)acetamide;
N-(2-(5-isopropyl-1,3,4-oxadiazol-2-yl)thiophen-3-yl)-2-(4-methoxyphenyl)acetamide;
N-(2-(5-methyl-1,3,4-oxadiazol-2-yl)thiophen-3-yl)-2-(naphthalen-1-yl)acetamide;
N-(4-methyl-3-(3-methyl-1,2,4-oxadiazol-5-yl)thiophen-2-yl)-2-(naphthalen-1-yl)acetamide;
N-(4-methyl-3-(3-methyl-1,2,4-oxadiazol-5-yl)thiophen-2-yl)-2-(4-(pyridin-4-yl)phenyl)acetamide;
N-(2-(3-methyl-1,2,4-oxadiazol-5-yl)thiophen-3-yl)-2-(naphthalen-1-yl)acetamide;
N-(4-(1H-1,2,4-triazol-5-yl)thiazol-5-yl)-2-(isoquinolin-5-yl)acetamide;
2-(isoquinolin-5-yl)-N-(4-(1-methyl-1H-1,2,4-triazol-5-yl)thiazol-5-yl)acetamide;
2-(2-pyridyl)-3-(1-naphthylacetylamino)thiophene;
N-(2-(1H-pyrazol-1-yl)thiophen-3-yl)-2-(4-methoxyphenyl)-acetamide;
2-(4-methoxyphenyl)-N-(2-(4-methyl-1H-pyrazol-1-yl)thiophen-3-yl)acetamide;
N-(2-(1H-pyrazol-3-yl)thiophen-3-yl)-2-(naphthalen-1-yl)acetamide;
N-(2-(1-methyl-1H-pyrazol-3-yl)thiophen-3-yl)-2-(naphthalen-1-yl)acetamide;
N-(2-(5-methyl-1H-pyrazol-3-yl)thiophen-3-yl)-2-(naphthalen-1-yl)acetamide;
N-(3-(2H-tetrazol-5-yl)thiophen-2-yl)-2-(4-methoxyphenyl)-acetamide;
2-(4-methoxyphenyl)-N-(3-(2-methyl-2H-tetrazol-5-yl)thiophen-2-yl)acetamide;
N-(3-(2-(methoxymethyl)-2H-tetrazol-5-yl)thiophen-2-yl)-2-(4-methoxyphenyl)acetamide;
N-(3-(1-(methoxymethyl)-1H-tetrazol-5-yl)thiophen-2-yl)-2-(4-methoxyphenyl)acetamide;
N-(2-(1-methyl-1H-imidazol-2-yl)thiophen-3-yl)-2-(naphthalen-1-yl)acetamide;
2-(4-methoxyphenyl)-N-(2-(1-methyl-1H-imidazol-4-yl)thiophen-3-yl)acetamide;
N-(2-(1H-imidazol-4-yl)thiophen-3-yl)-2-(4-methoxyphenyl)-acetamide;
N-(2-(1H-imidazol-4-yl)thiophen-3-yl)-2-(2-oxo-3,4-dihydroquinolin-1(2H)-yl)acetamide;
2-(4-methoxyphenyl)-N-(2-(2-methyl-1H-imidazol-4-yl)thiophen-3-yl)acetamide;
N-(2-(2-methyl-1H-imidazol-4-yl)thiophen-3-yl)-2-(2-oxo-3,4-dihydroquinolin-1(2H)-yl)acetamide;
N-(2-(1H-imidazol-1-yl)thiophen-3-yl)-2-(naphthalen-1-yl)acetamide;
2-(4-methoxyphenyl)-N-(2-(4-methyl-1H-imidazol-1-yl)thiophen-3-yl)acetamide;
2-(4-methoxyphenyl)-N-(2-(pyrazin-2-yl)thiophen-3-yl)acetamide; 2-(isoquinolin-5-yl)-N-(4-(pyrazin-2-yl)thiazol-5-yl)acetamide;
N-(4,4′-bithiazol-5-yl)-2-(isoquinolin-5-yl)acetamide;
2-(4-methoxyphenyl)-N-(2-(2-oxooxazolidin-3-yl)thiophen-3-yl)acetamide;
2-(7-bromo-2-oxo-3,4-dihydroquinolin-1(2H)-yl)-N-(4-bromo-3-(1H-1,2,4-triazol-5-yl)thiophen-2-yl)acetamide;
N-(4-bromo-3-(4H-1,2,4-triazol-3-yl)thiophen-2-yl)-2-(quinolin-4-yl)acetamide;
N-(4-bromo-3-(1H-1,2,4-triazol-5-yl)thiophen-2-yl)-2-(2-oxo-6-(trifluoromethyl)quinolin-1(2H)-yl)acetamide;
N-(4-bromo-3-(1H-1,2,4-triazol-5-yl)thiophen-2-yl)-2-(8-fluoroisoquinolin-5-yl)acetamide;
N-(4-bromo-3-(1H-1,2,4-triazol-5-yl)thiophen-2-yl)-2-(2-oxo-1,6-naphthyridin-1(2H)-yl)acetamide;
N-(4-bromo-3-(1H-1,2,4-triazol-5-yl)thiophen-2-yl)-2-(8-fluoroquinolin-5-yl)acetamide;
N-(4-bromo-3-(4H-1,2,4-triazol-3-yl)thiophen-2-yl)-2-(7-(trifluoromethyl)quinolin-5-yl)acetamide;
N-(4-bromo-3-(4H-1,2,4-triazol-3-yl)thiophen-2-yl)-2-(5-(trifluoromethyl)quinolin-7-yl)acetamide;
N-(4-bromo-3-(1H-1,2,4-triazol-5-yl)thiophen-2-yl)-2-(2-oxo-1,5-naphthyridin-1(2H)-yl)acetamide;
N-(4-bromo-3-(1H-1,2,4-triazol-5-yl)thiophen-2-yl)-2-(2-oxo-3,4-dihydro-1,6-naphthyridin-1(2H)-yl)acetamide;
N-(4-bromo-3-(1H-1,2,4-triazol-5-yl)thiophen-2-yl)-2-(2-oxo-3,4-dihydro-1,5-naphthyridin-1(2H)-yl)acetamide;
N-(4-bromo-3-(1H-1,2,4-triazol-5-yl)thiophen-2-yl)-2-(6-chloro-2-oxoquinolin-1(2H)-yl)acetamide;
N-(4-bromo-3-(1H-1,2,4-triazol-5-yl)thiophen-2-yl)-2-(5-fluoro-2-oxoquinolin-1(2H)-yl)acetamide;
N-(4-bromo-3-(1H-1,2,4-triazol-3-yl)thiophen-2-yl)-2-(3-fluoroquinolin-8-yl)acetamide;
N-(4-bromo-3-(1H-1,2,4-triazol-5-yl)thiophen-2-yl)-2-(2-oxo-6-(trifluoromethoxy)quinolin-1(2H)-yl)acetamide;
N-(4-bromo-3-(4H-1,2,4-triazol-3-yl)thiophen-2-yl)-2-(isoquinolin-4-yl)acetamide;
N-(5-chloro-3-(1H-1,2,4-triazol-5-yl)thiophen-2-yl)-2-(2-oxo-6-(trifluoromethyl)quinolin-1(2H)-yl)acetamide;
N-(4-bromo-3-(1H-1,2,4-triazol-3-yl)thiophen-2-yl)-2-(3-fluoroquinolin-5-yl)acetamide;
N-(4-bromo-3-(1H-1,2,4-triazol-5-yl)thiophen-2-yl)-2-(2-oxo-7-(trifluoromethoxy)quinolin-1(2H)-yl)acetamide;
N-(4-bromo-3-(1H-1,2,4-triazol-5-yl)thiophen-2-yl)-2-(7-cyano-2-oxoquinolin-1(2H)-yl)acetamide;
N-(4-bromo-3-(1H-1,2,4-triazol-5-yl)thiophen-2-yl)-2-(isoquinolin-8-yl)acetamide;
N-(4-bromo-3-(1H-1,2,4-triazol-5-yl)thiophen-2-yl)-2-(6-cyano-2-oxoquinolin-1(2H)-yl)acetamide;
N-(4-bromo-3-(1H-1,2,4-triazol-5-yl)thiophen-2-yl)-2-(quinolin-8-yl)acetamide;
N-(4-bromo-3-(1H-1,2,4-triazol-5-yl)thiophen-2-yl)-2-(2-oxo-5-(trifluoromethyl)quinolin-1(2H)-yl)acetamide;
N-(4-bromo-3-(1H-1,2,4-triazol-5-yl)thiophen-2-yl)-2-(2-oxo-6-(trifluoromethyl)-3,4-dihydroquinolin-1(2H)-yl)acetamide;
N-(4-cyano-3-(1-methyl-1H-1,2,4-triazol-3-yl)thiophen-2-yl)-2-(quinolin-5-yl)acetamide;
N-(4-bromo-3-(1H-1,2,4-triazol-3-yl)thiophen-2-yl)-2-(2-(trifluoromethyl)quinolin-7-yl)acetamide;
N-(4-bromo-3-(1H-1,2,4-triazol-5-yl)thiophen-2-yl)-2-(7-fluoroquinolin-5-yl)acetamide;
N-(4-bromo-3-(1H-1,2,4-triazol-3-yl)thiophen-2-yl)-2-(3-(trifluoromethyl)quinolin-5-yl)acetamide;
N-(4-bromo-3-(1H-1,2,4-triazol-5-yl)thiophen-2-yl)-2-(6-fluoroquinolin-5-yl)acetamide;
N-(4-bromo-3-(1H-1,2,4-triazol-5-yl)thiophen-2-yl)-2-(6-fluoroquinolin-7-yl)acetamide;
N-(4-bromo-3-(1H-1,2,4-triazol-5-yl)thiophen-2-yl)-2-(6-ethynyl-2-oxoquinolin-1(2H)-yl)acetamide;
N-(4-bromo-3-(1H-1,2,4-triazol-5-yl)thiophen-2-yl)-2-(2-oxo-7-(trifluoromethyl)-1,6-naphthyridin-1(2H)-yl)acetamide;
N-(4-bromo-3-(1H-1,2,4-triazol-5-yl)thiophen-2-yl)-2-(5-oxopyrazolo[1,5-a]pyrimidin-4(5H)-yl)acetamide;
N-(4-bromo-3-(1H-1,2,4-triazol-3-yl)thiophen-2-yl)-2-(3-(trifluoromethyl)quinolin-8-yl)acetamide;
N-(4-bromo-3-(1H-1,2,4-triazol-5-yl)thiophen-2-yl)-2-(6-methylimidazo[2,1-b]thiazol-3-yl)acetamide;
2-(2-oxo-6-(trifluoromethyl)quinolin-1(2H)-yl)-N-(2-(thiazol-4-yl)thiophen-3-yl)acetamide;
N-(4-cyano-3-(pyrazin-2-yl)thiophen-2-yl)-2-(quinolin-5-yl)acetamide;
2-(2-oxo-1,6-naphthyridin-1(2H)-yl)-N-(2-(thiazol-4-yl)thiophen-3-yl)acetamide;
N-(4-bromo-3-(4H-1,2,4-triazol-3-yl)thiophen-2-yl)-2-(3,3-difluoro-2-oxoindolin-1-yl)acetamide;
2-(benzo[d]thiazol-7-yl)-N-(4-bromo-3-(1H-1,2,4-triazol-5-yl)thiophen-2-yl)acetamide;
N-(4-chloro-3-(1H-1,2,4-triazol-5-yl)thiophen-2-yl)-2-(2-oxo-6-(trifluoromethyl)quinolin-1(2H)-yl)acetamide;
N-(4-cyano-3-(oxazol-2-yl)thiophen-2-yl)-2-(2-oxo-6-(trifluoromethyl)quinolin-1(2H)-yl)acetamide;
N-(4-bromo-3-(1H-1,2,4-triazol-5-yl)thiophen-2-yl)-2-(2-oxo-6-(trifluoromethyl)-1,5-naphthyridin-1(2H)-yl)acetamide;
N-(3-(1,2,4-oxadiazol-3-yl)thiophen-2-yl)-2-(6,7-difluoro-2-oxoquinolin-1(2H)-yl)acetamide;
N-(4-cyano-3-(thiazol-2-yl)thiophen-2-yl)-2-(2-oxo-6-(trifluoromethyl)quinolin-1(2H)-yl)acetamide;
N-(4-bromo-3-(1H-1,2,4-triazol-5-yl)thiophen-2-yl)-2-(5-oxo-2-(trifluoromethyl)pyrazolo[1,5-a]pyrimidin-4(5H)-yl)acetamide;
N-(4-cyano-3-(thiazol-4-yl)thiophen-2-yl)-2-(2-oxo-6-(trifluoromethyl)quinolin-1(2H)-yl)acetamide;
N-(4-bromo-3-(1H-1,2,4-triazol-5-yl)thiophen-2-yl)-2-(imidazo[1,2-a]pyridin-5-yl)acetamide;
N-(4-bromo-3-(1H-1,2,4-triazol-5-yl)thiophen-2-yl)-2-(7-fluoro-2-oxo-6-(trifluoromethyl)quinolin-1(2H)-yl)acetamide;
N-(4-bromo-3-(1-methyl-1H-1,2,4-triazol-3-yl)thiophen-2-yl)-2-(2-oxo-3,4-dihydro-1,5-naphthyridin-1(2H)-yl)acetamide;
N-(4-cyano-3-(thiazol-5-yl)thiophen-2-yl)-2-(2-oxo-6-(trifluoromethyl)quinolin-1(2H)-yl)acetamide;
N-(4-cyano-3-(1H-1,2,3-triazol-1-yl)thiophen-2-yl)-2-(2-oxo-6-triazol-1-yl)-4-cyanothiophen-2-yl)-2-(2-oxo-6-(trifluoromethyl)quinolin-1(2H)-yl)acetamide;
N-(3-(1H-benzo[d][1,2,3]triazol-1-yl)-4-cyanothiophen-2-yl)-2-(2-oxo-6-(trifluoromethyl)quinolin-1(2H)-yl)acetamide;
N-(4-bromo-3-(1H-1,2,4-triazol-5-yl)thiophen-2-yl)-2-(6-fluoroisoquinolin-5-yl)acetamide;
N-(4-bromo-3-(1H-1,2,4-triazol-5-yl)thiophen-2-yl)-2-(6-fluoroisoquinolin-7-yl)acetamide;
N-(4-cyano-3-(2H-1,2,3-triazol-2-yl)thiophen-2-yl)-2-(2-oxo-6-(trifluoromethyl)quinolin-1(2H)-yl)acetamide;
N-(4-chloro-3-(1H-1,2,4-triazol-3-yl)thiophen-2-yl)-2-(2-oxo-3,4-dihydro-1,5-naphthyridin-1(2H)-yl)acetamide;
N-(4-cyano-3-(thiazol-2-yl)thiophen-2-yl)-2-(2-oxo-3,4-dihydro-1,5-naphthyridin-1(2H)-yl)acetamide;
N-(4-chloro-3-(1-methyl-1H-1,2,4-triazol-3-yl)thiophen-2-yl)-2-(2-oxo-3,4-dihydro-1,5-naphthyridin-1(2H)-yl)acetamide;
N-(4-chloro-3-(1H-1,2,4-triazol-5-yl)thiophen-2-yl)-2-(5-oxopyrazolo[1,5-a]pyrimidin-4(5H)-yl)acetamide;
N-(4-bromo-3-(oxazol-2-yl)thiophen-2-yl)-2-(2-oxo-3,4-dihydro-1,5-naphthyridin-1(2H)-yl)acetamide;
N-(4-bromo-3-(thiazol-2-yl)thiophen-2-yl)-2-(2-oxo-3,4-dihydro-1,5-naphthyridin-1(2H)-yl)acetamide;
N-(4-chloro-3-(thiazol-2-yl)thiophen-2-yl)-2-(2-oxo-3,4-dihydro-1,5-naphthyridin-1(2H)-yl)acetamide;
N-(3-(benzo[d]thiazol-2-yl)-4-cyanothiophen-2-yl)-2-(2-oxo-3,4-dihydro-1,5-naphthyridin-1(2H)-yl)acetamide;
N-(4-chloro-3-(3-methyl-1H-1,2,4-triazol-5-yl)thiophen-2-yl)-2-(2-oxo-3,4-dihydro-1,5-naphthyridin-1(2H)-yl)acetamide;
N-(4-chloro-3-(1-(3-(dimethylamino)propyl)-1H-1,2,4-triazol-3-yl)thiophen-2-yl)-2-(2-oxo-6-(trifluoromethyl)quinolin-1(2H)-yl)acetamide;
N-(4-chloro-3-(1-(3-(4-methylpiperazin-1-yl)propyl)-1H-1,2,4-triazol-3-yl)thiophen-2-yl)-2-(2-oxo-6-(trifluoromethyl)quinolin-1(2H)-yl)acetamide;
N-(4-chloro-3-(3-ethyl-1H-1,2,4-triazol-5-yl)thiophen-2-yl)-2-(2-oxo-3,4-dihydro-1,5-naphthyridin-1(2H)-yl)acetamide;
N-(4-chloro-3-(1-(2-(dimethylamino)ethyl)-1H-1,2,4-triazol-3-yl)thiophen-2-yl)-2-(2-oxo-6-(trifluoromethyl)quinolin-1(2H)-yl)acetamide;
N-(4-chloro-3-(1H-1,2,4-triazol-3-yl)thiophen-2-yl)-N-(2-(dimethylamino)ethyl)-2-(2-oxo-6-(trifluoromethyl)quinolin-1(2H)-yl)acetamide;
N-(4-cyano-3-(1H-1,2,4-triazol-3-yl)thiophen-2-yl)-2-(2-oxo-3,4-dihydro-1,5-naphthyridin-1(2H)-yl)acetamide;
N-(4-chloro-3-(1-methyl-1H-1,2,4-triazol-3-yl)thiophen-2-yl)-2-(5-oxopyrazolo[1,5-a]pyrimidin-4(5H)-yl)acetamide;
N-(4-chloro-3-(1-(3-morpholinopropyl)-1H-1,2,4-triazol-3-yl)thiophen-2-yl)-2-(2-oxo-6-(trifluoromethyl)quinolin-1(2H)-yl)acetamide;
N-(4-chloro-3-(1H-1,2,4-triazol-3-yl)thiophen-2-yl)-2-(2-oxo-6-(trifluoromethyl)quinolin-1(2H)-yl)-N-(3-(pyrrolidin-1-yl)propyl)acetamide;
N-(4-chloro-3-(1-(3-(pyrrolidin-1-yl)propyl)-1H-1,2,4-triazol-3-yl)thiophen-2-yl)-2-(2-oxo-6-(trifluoromethyl)quinolin-1(2H)-yl)acetamide;
N-(4-chloro-3-(1-methyl-1H-1,2,4-triazol-3-yl)thiophen-2-yl)-2-(2-oxo-6-(trifluoromethyl)-1,5-naphthyridin-1(2H)-yl)acetamide;
N-(4-chloro-3-(1H-1,2,4-triazol-5-yl)thiophen-2-yl)-2-(8-(trifluoromethyl)quinolin-5-yl)acetamide;
N-(4-bromo-3-(2H-1,2,3-triazol-2-yl)thiophen-2-yl)-2-(2-oxo-6-(trifluoromethyl)quinolin-1(2H)-yl)acetamide;
N-(4-chloro-3-(1H-1,2,4-triazol-5-yl)thiophen-2-yl)-2-(2-oxo-6-(trifluoromethyl)-1,5-naphthyridin-1(2H)-yl)acetamide;
N-(4-chloro-3-(3-isopropyl-1H-1,2,4-triazol-5-yl)thiophen-2-yl)-2-(2-oxo-3,4-dihydro-1,5-naphthyridin-1(2H)-yl)acetamide;
2-(6-bromo-2-oxoquinolin-1(2H)-yl)-N-(4-bromo-3-(1H-1,2,4-triazol-3-yl)thiophen-2-yl)acetamide;
N-(4-cyano-3-(1-methyl-1H-1,2,4-triazol-3-yl)thiophen-2-yl)-2-(2-oxo-3,4-dihydro-1,5-naphthyridin-1(2H)-yl)acetamide;
2-(6-bromo-2-oxoquinolin-1(2H)-yl)-N-(4-chloro-3-(1H-1,2,4-triazol-3-yl)thiophen-2-yl)acetamide;
N-(4-chloro-3-(1H-1,2,4-triazol-3-yl)thiophen-2-yl)-2-(6-cyano-2-oxoquinolin-1(2H)-yl)acetamide;
N-(4-chloro-3-(1H-1,2,4-triazol-5-yl)thiophen-2-yl)-2-(5-oxo-2-(trifluoromethyl)pyrazolo[1,5-a]pyrimidin-4(5H)-yl)acetamide;
N-(4-chloro-3-(1H-1,2,4-triazol-3-yl)thiophen-2-yl)-2-(2-oxo-7-(trifluoromethyl)-1,6-naphthyridin-1(2H)-yl)acetamide;
N-(4-chloro-3-(1-methyl-1H-1,2,4-triazol-3-yl)thiophen-2-yl)-2-(5-oxo-2-(trifluoromethyl)pyrazolo[1,5-a]pyrimidin-4(5H)-yl)acetamide; and
N-(4-chloro-3-(1-methyl-1H-1,2,4-triazol-3-yl)thiophen-2-yl)-2-(2-oxo-7-(trifluoromethyl)-1,6-naphthyridin-1(2H)-yl)acetamide,

or a pharmaceutically acceptable salt thereof.

### In Vitro Activities

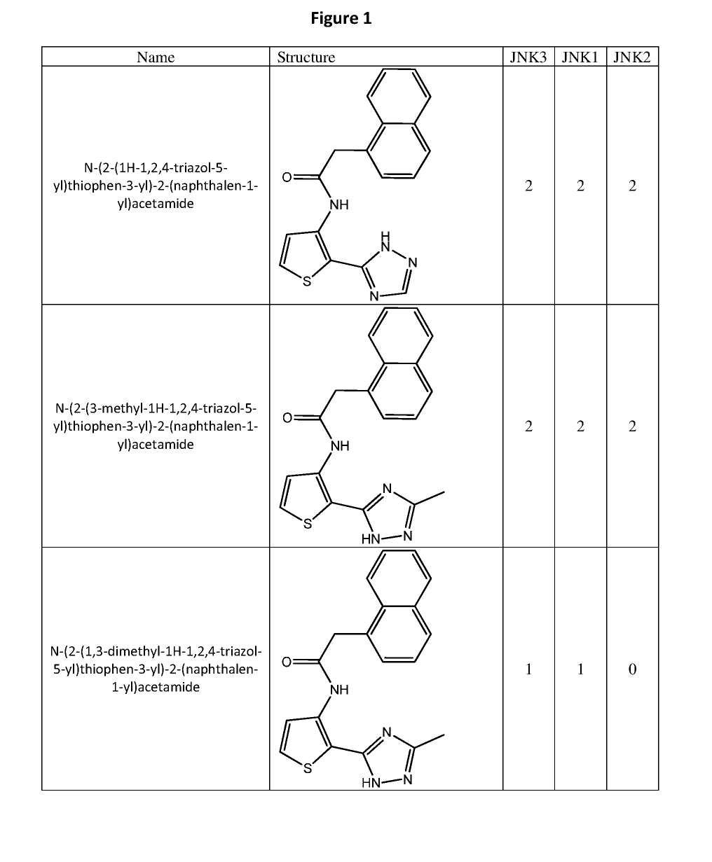

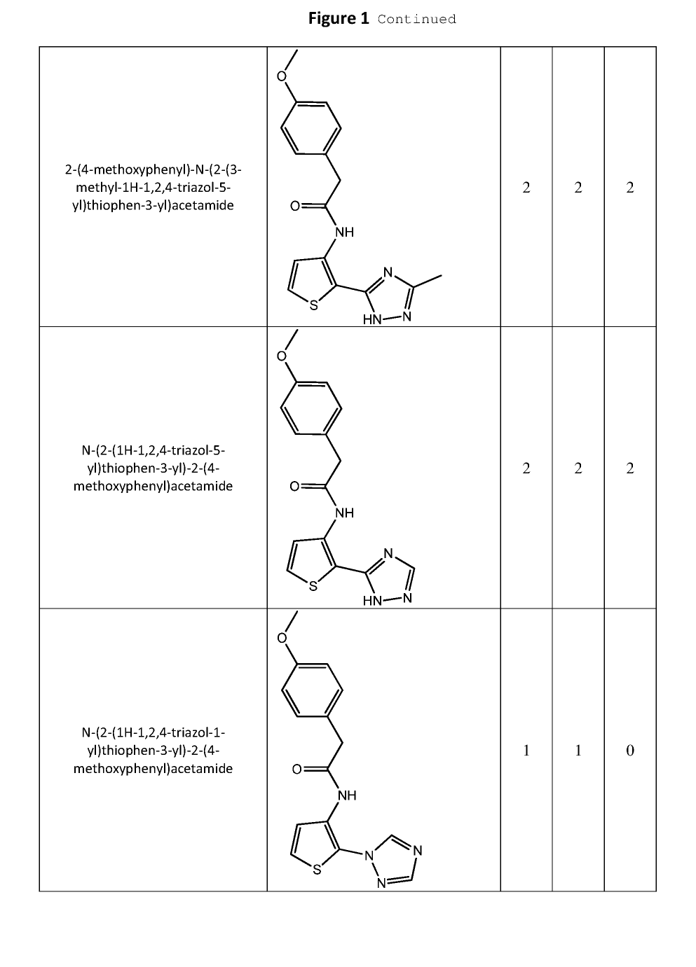

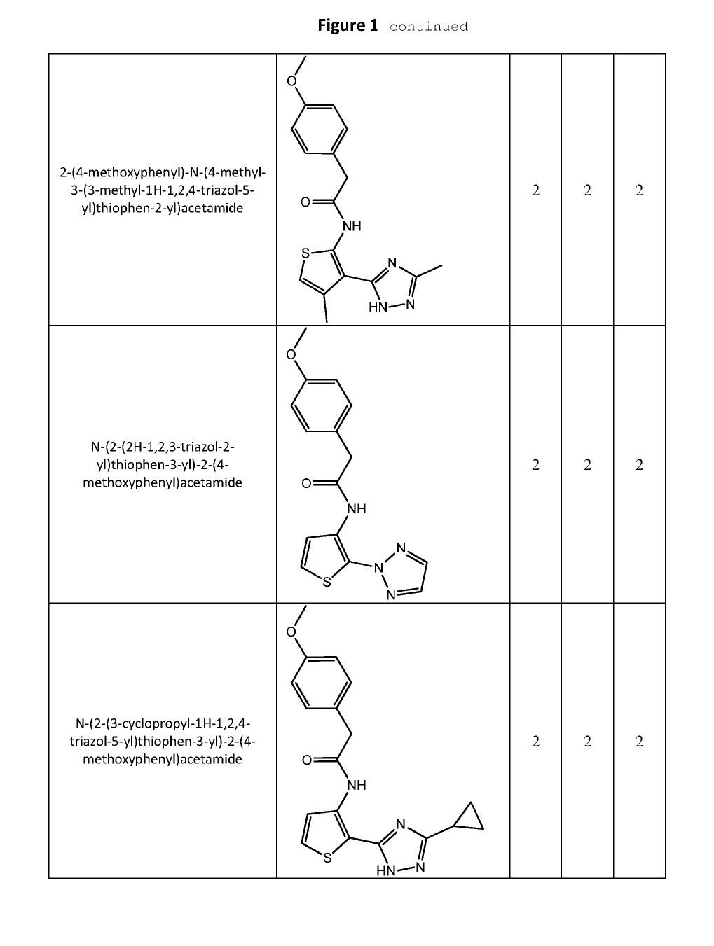

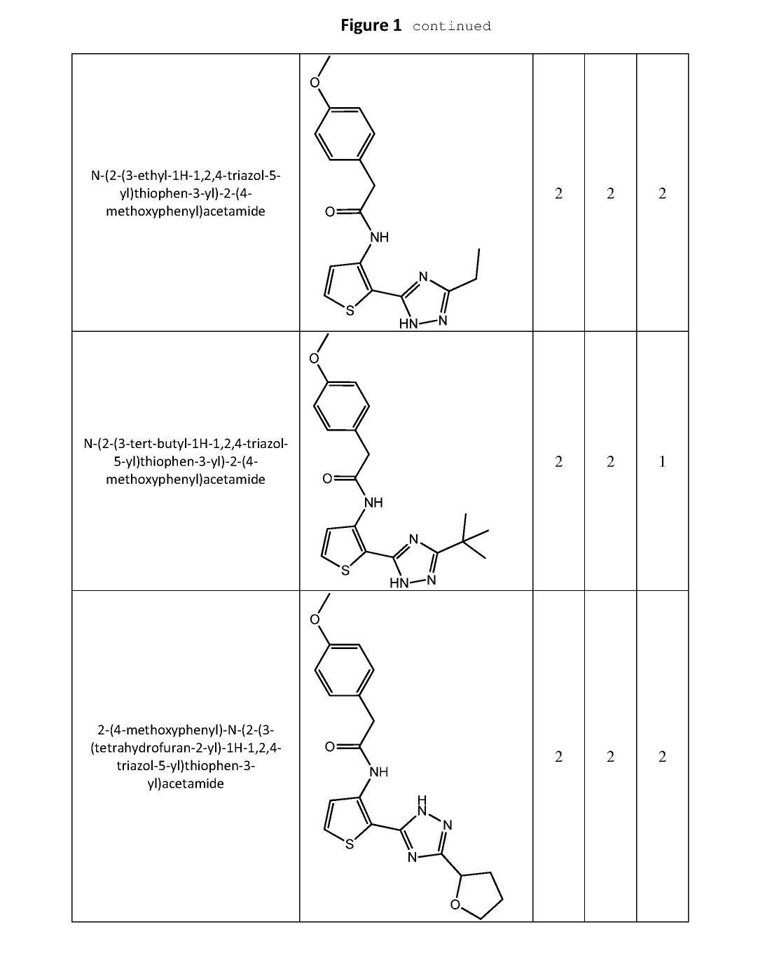

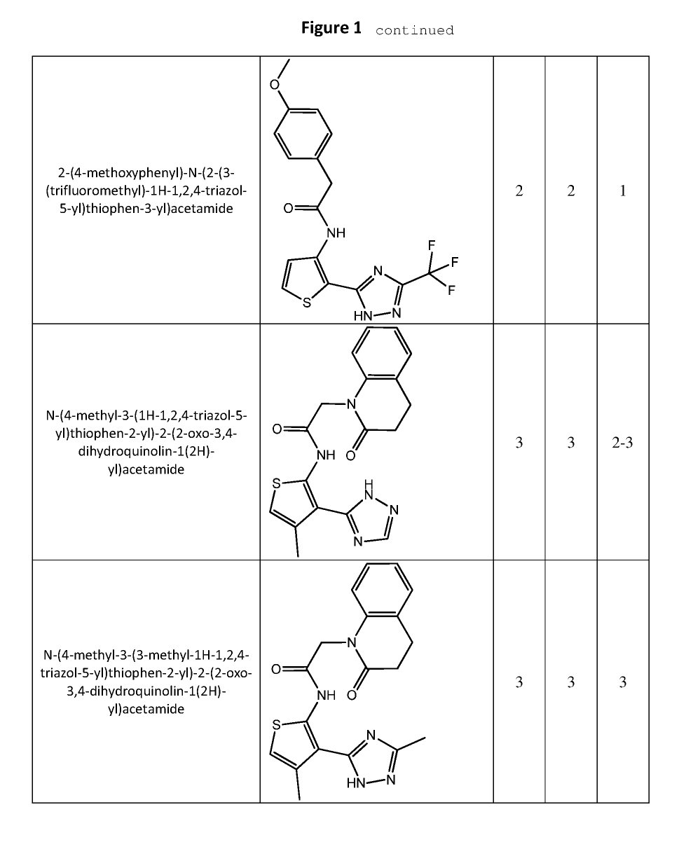

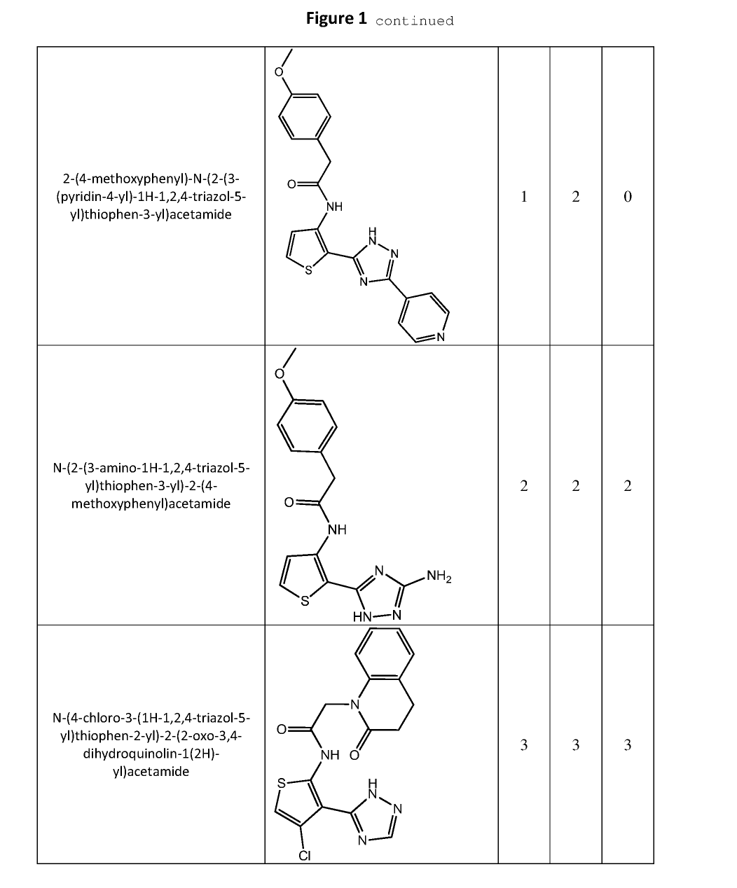

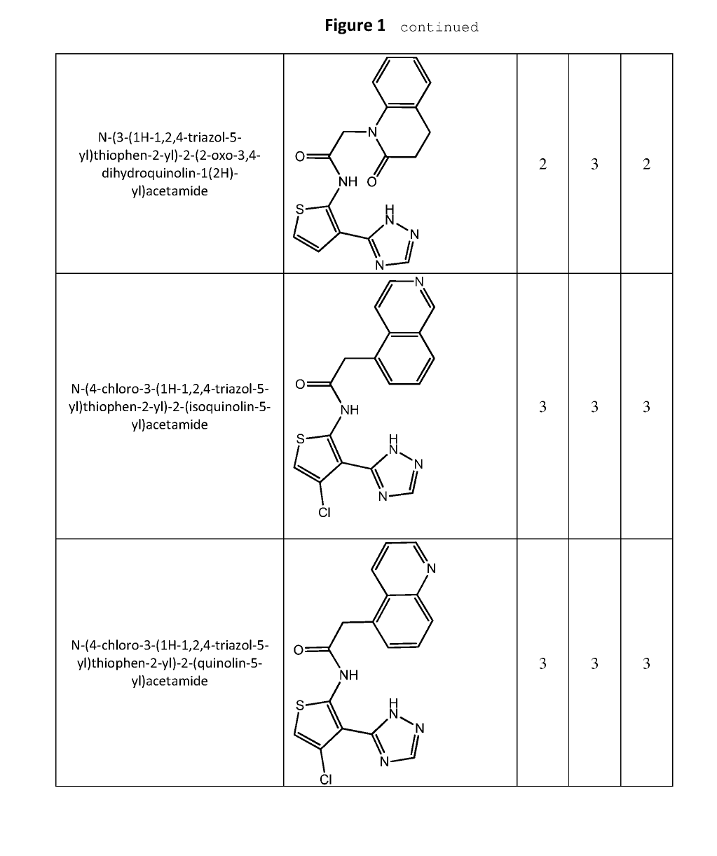

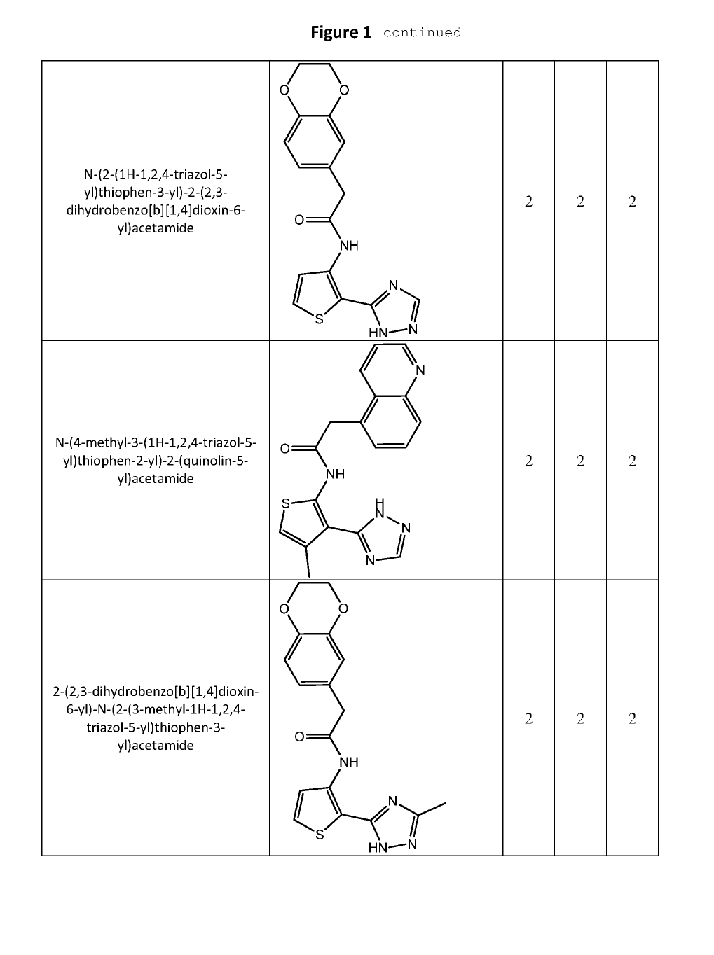

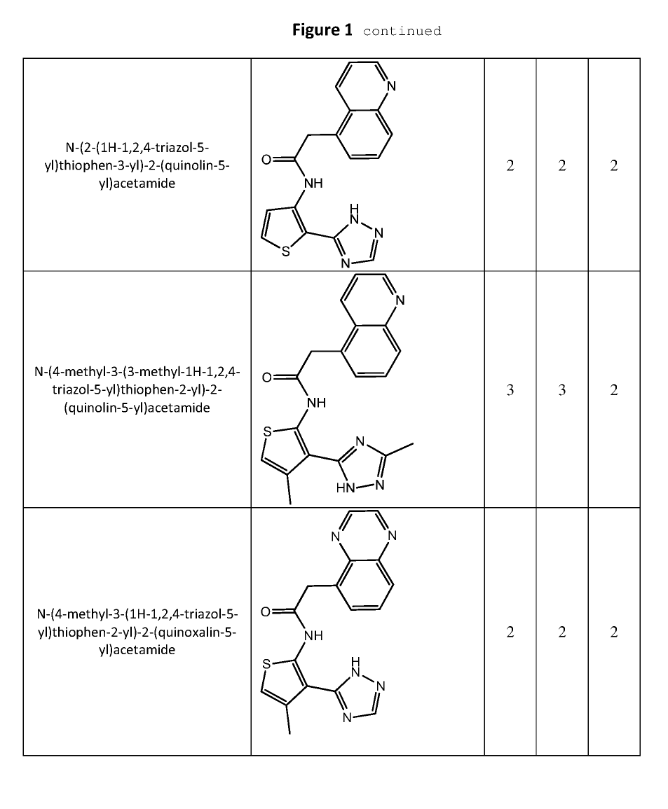

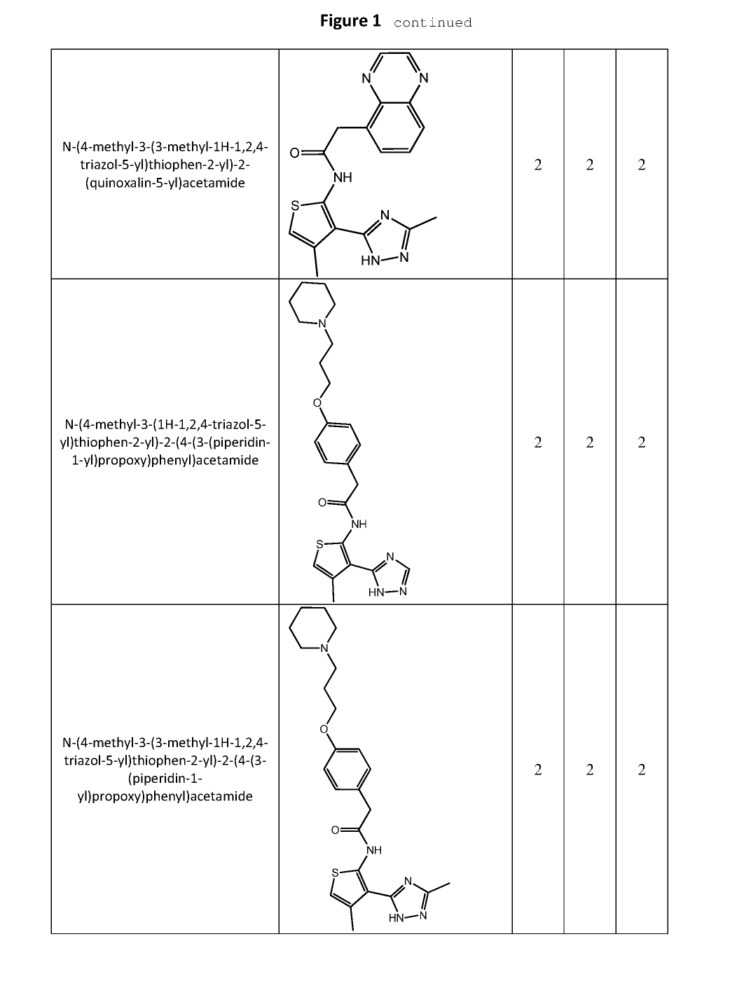

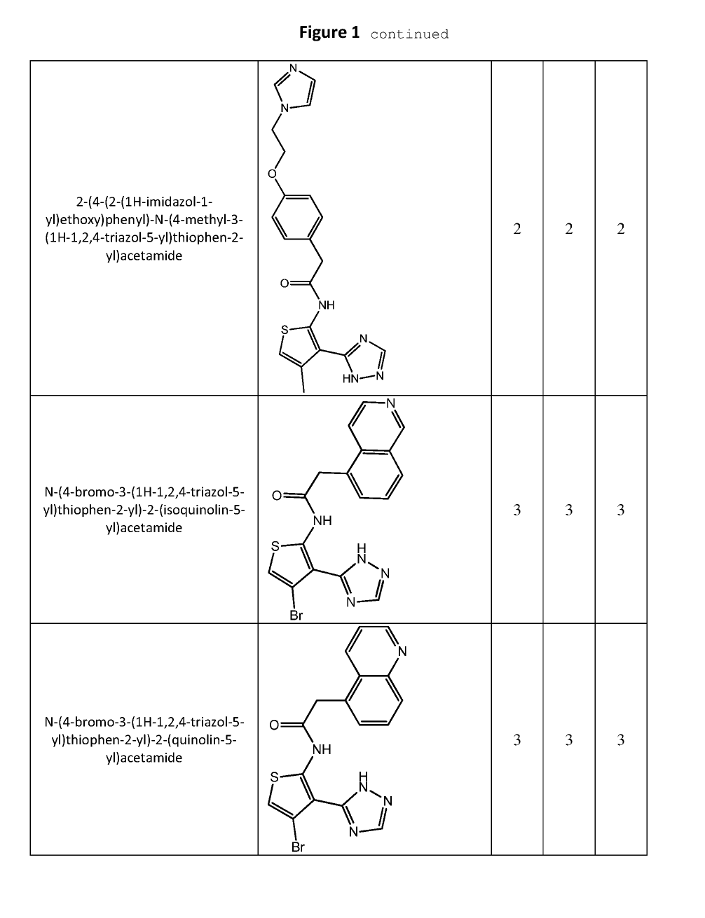

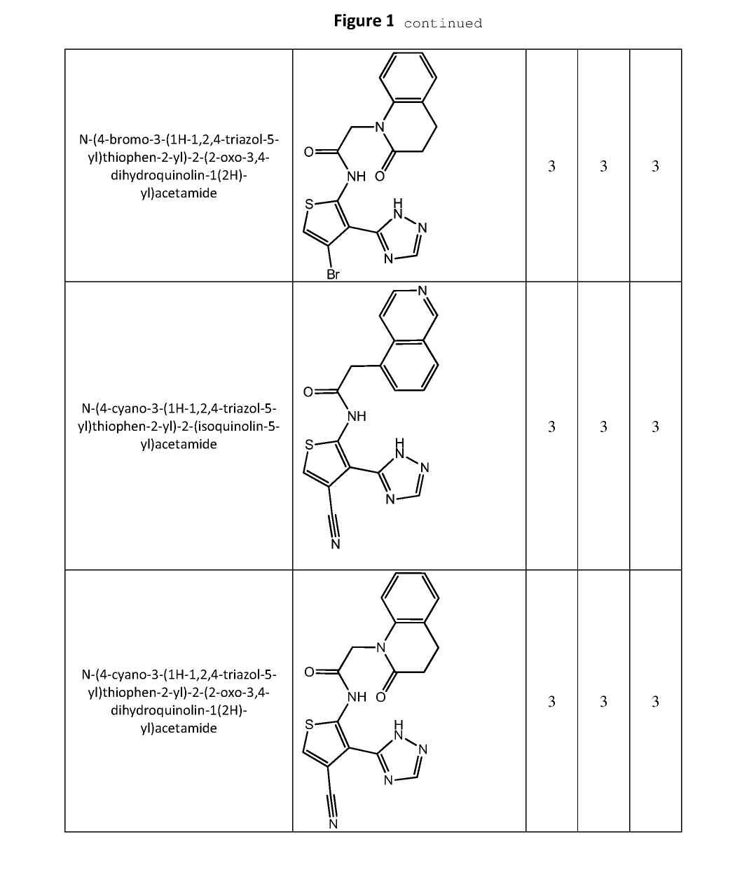

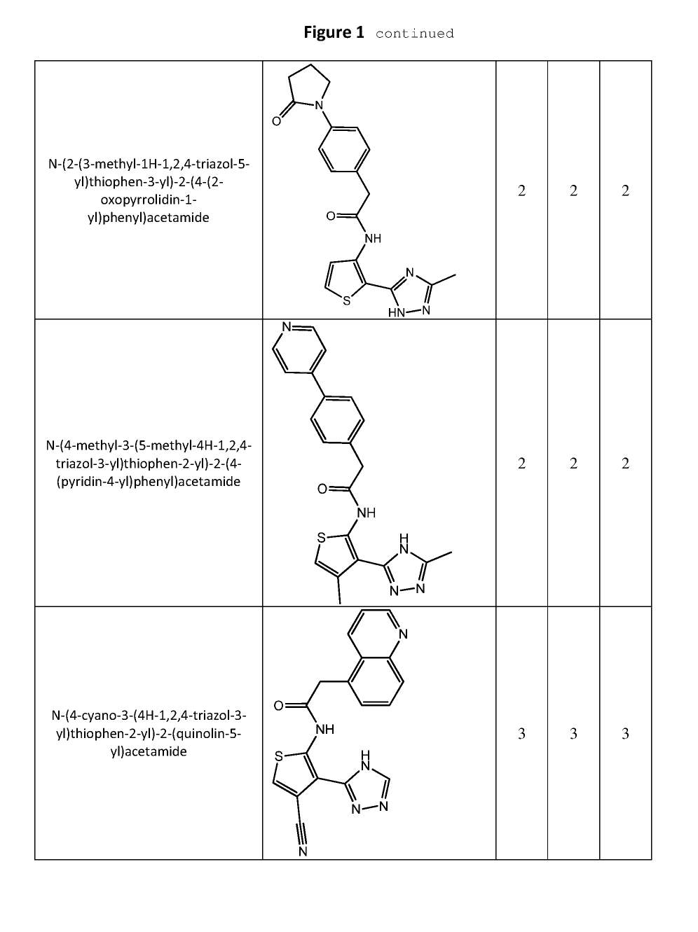

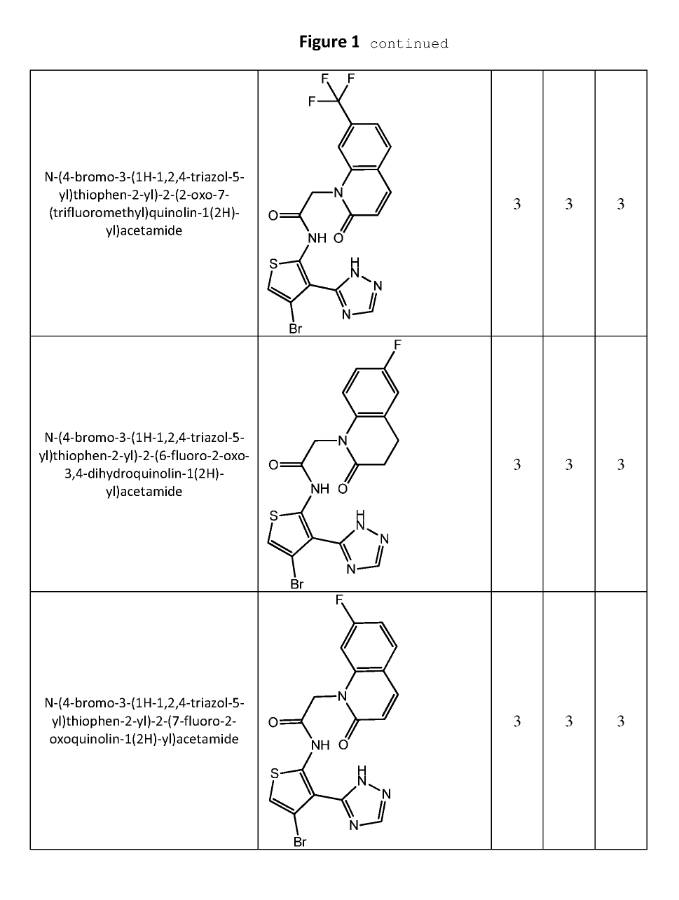

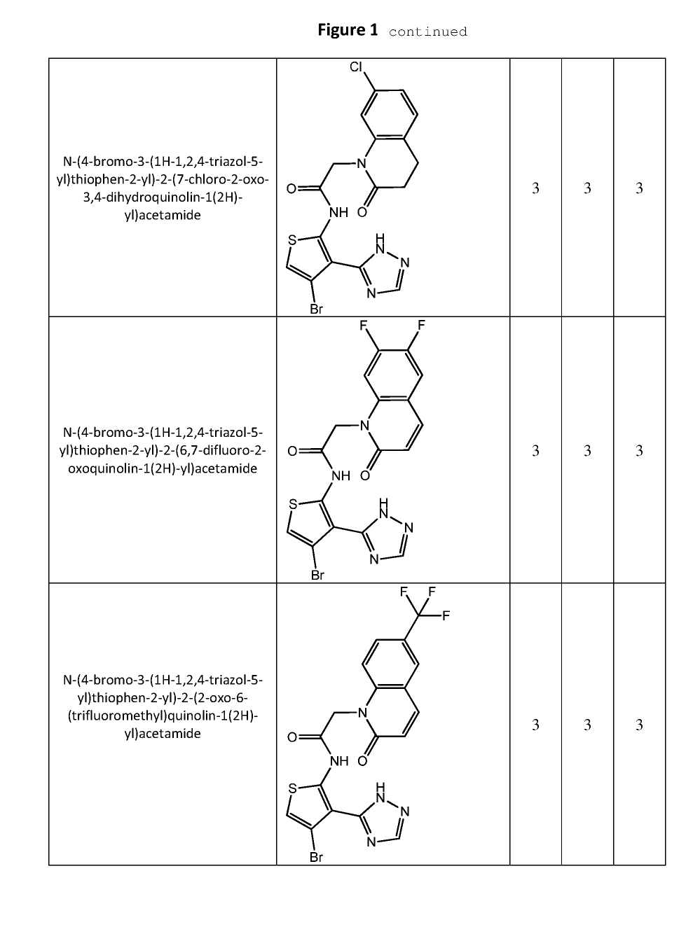

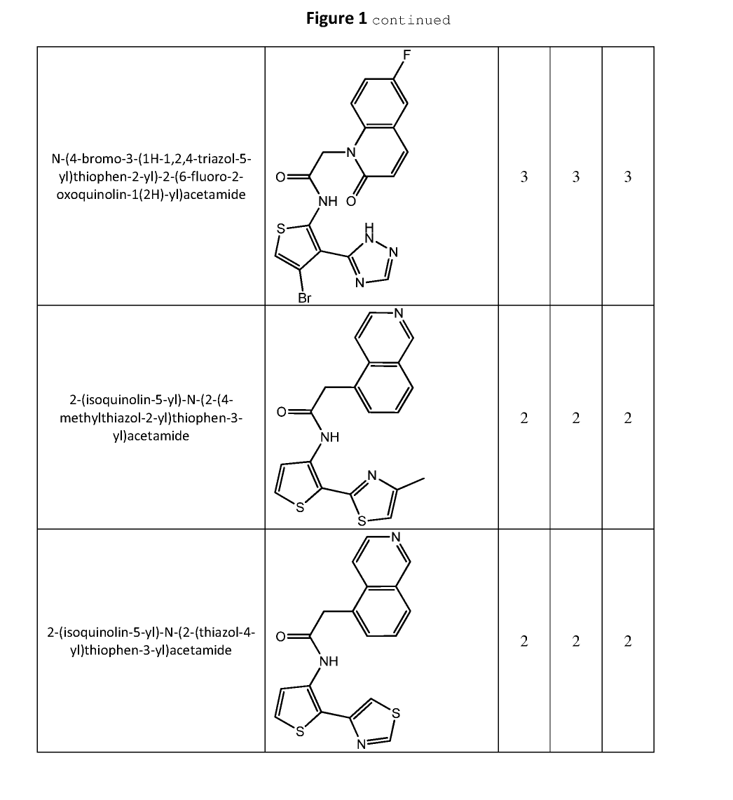

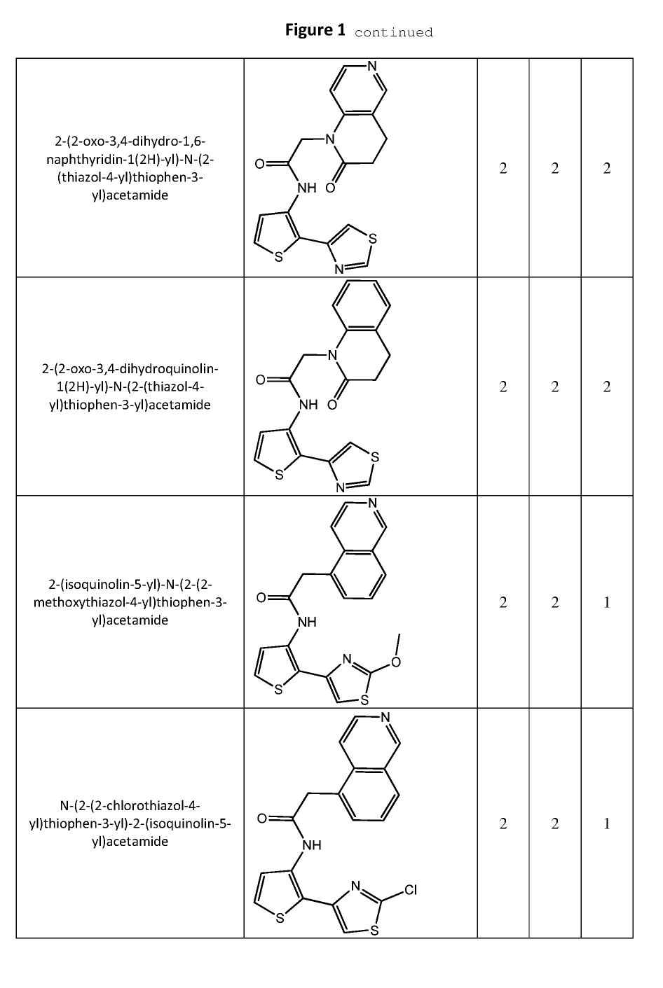

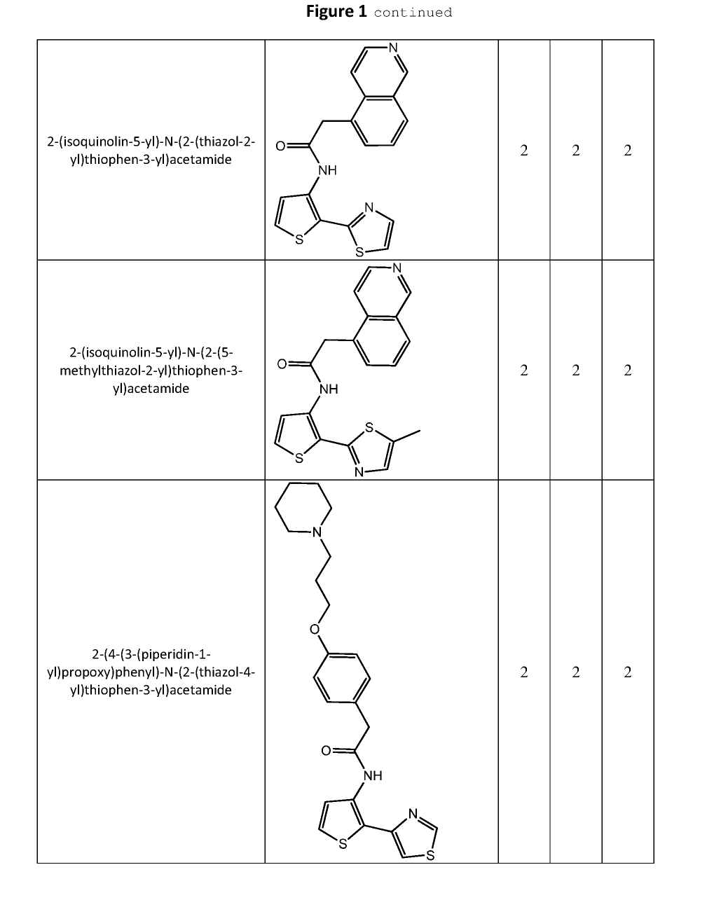

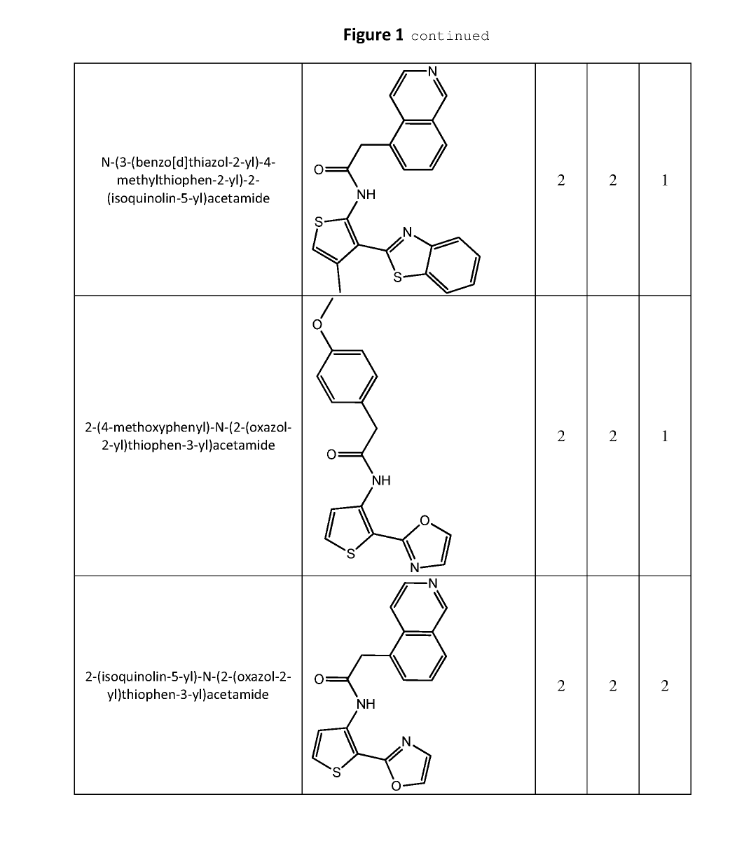

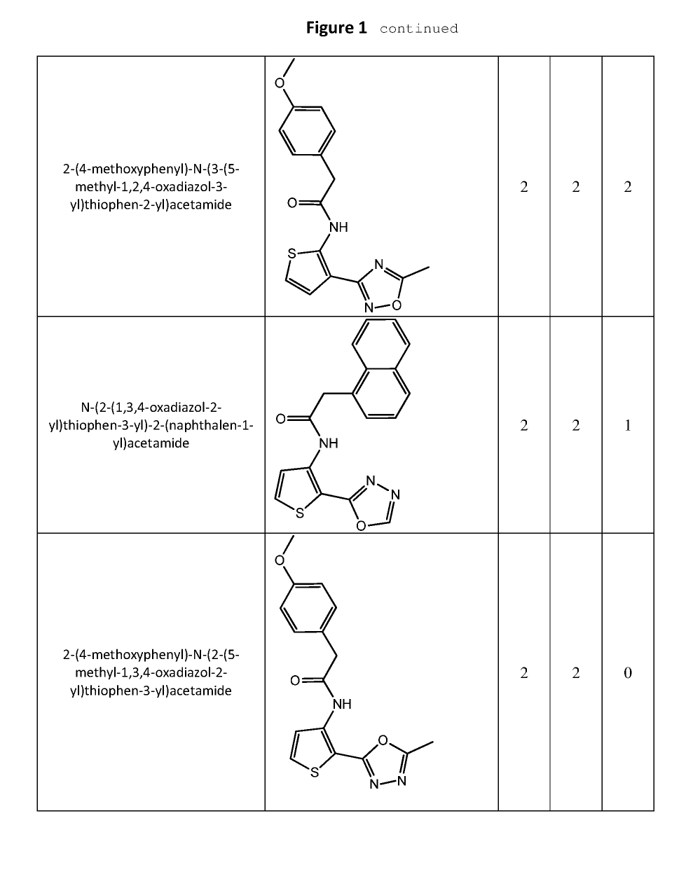

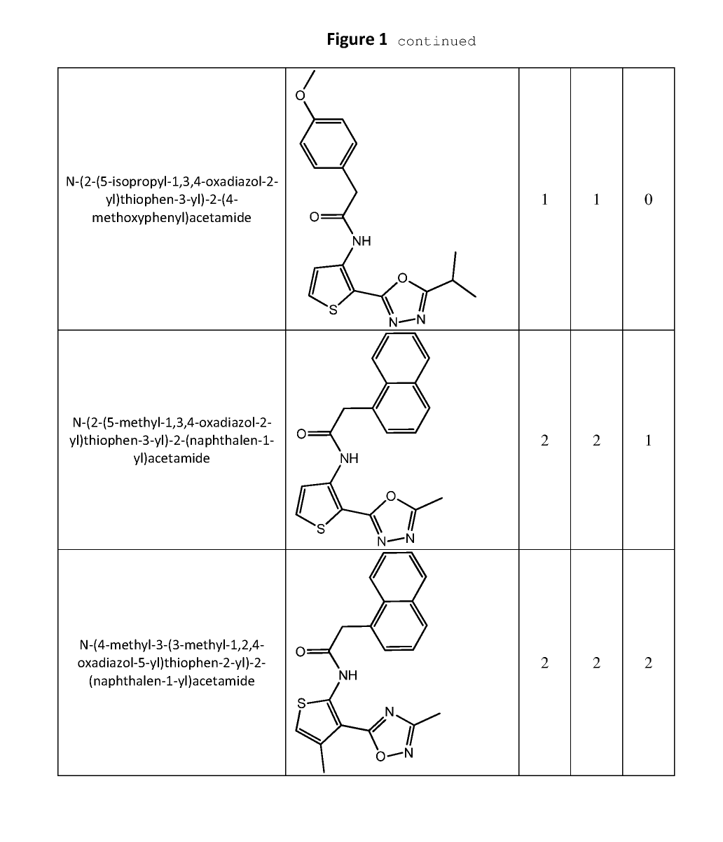

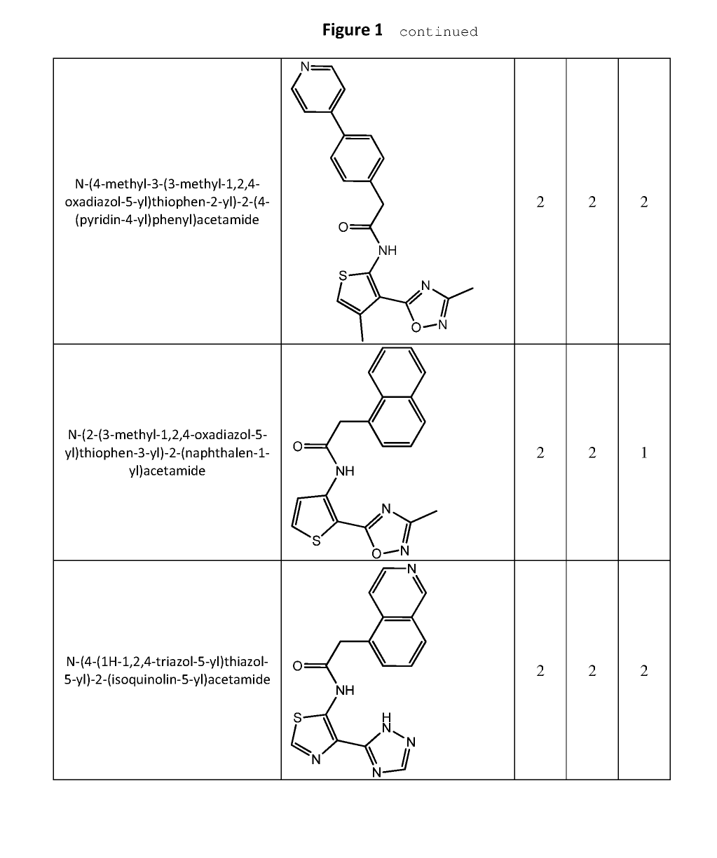

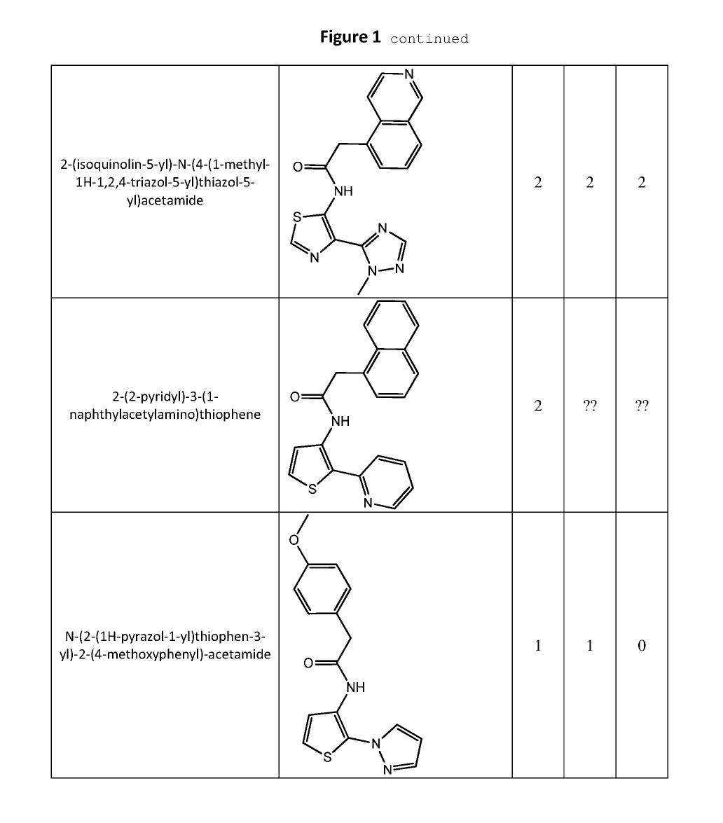

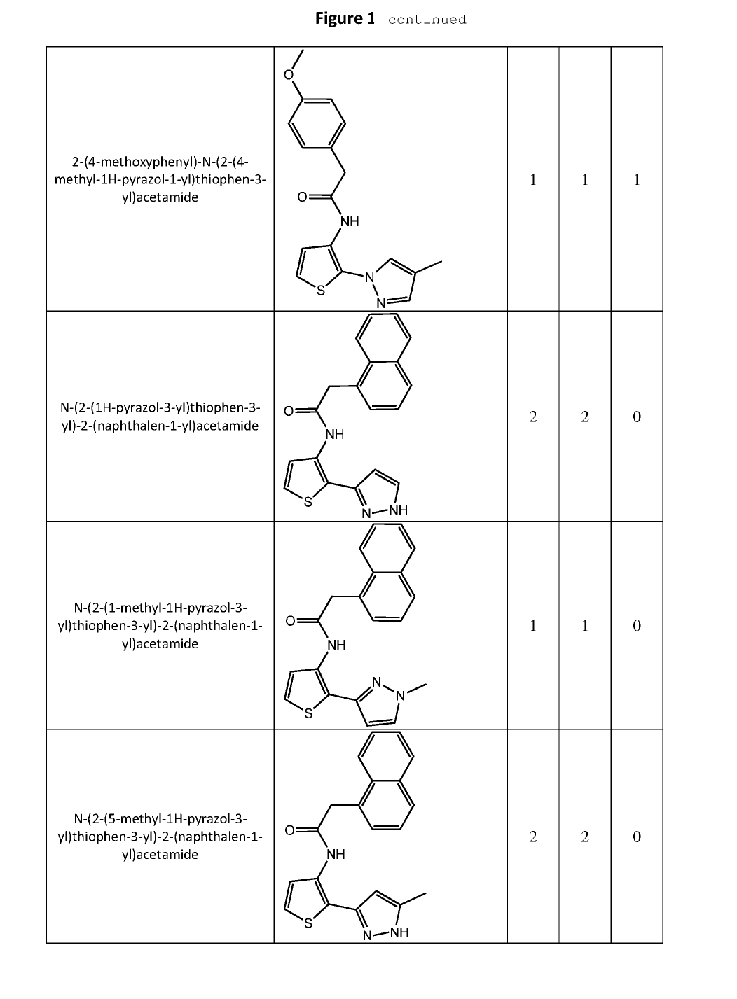

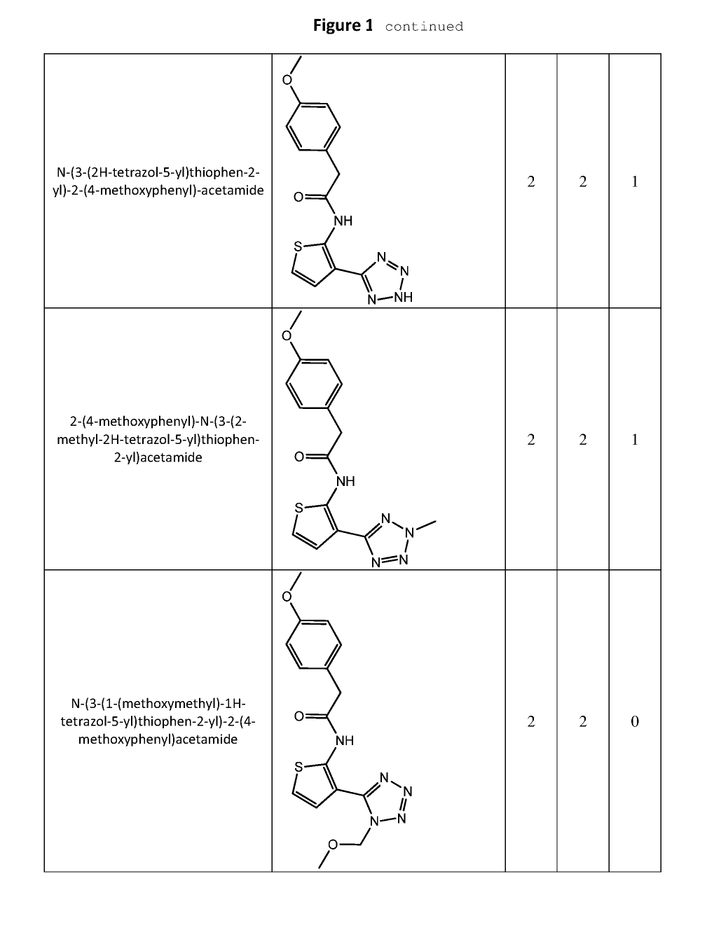

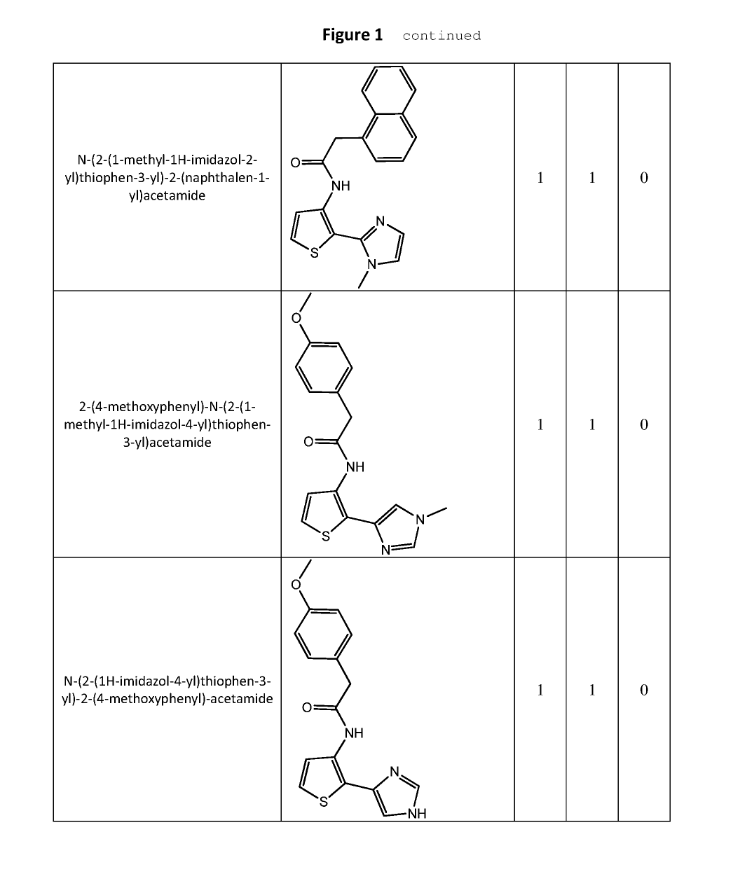

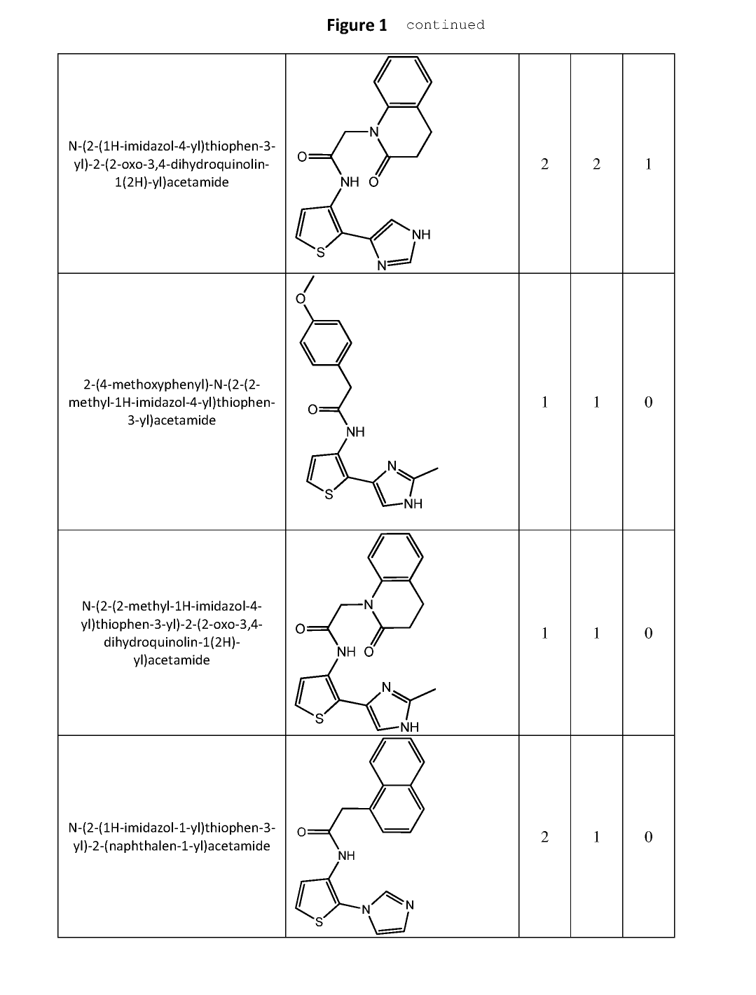

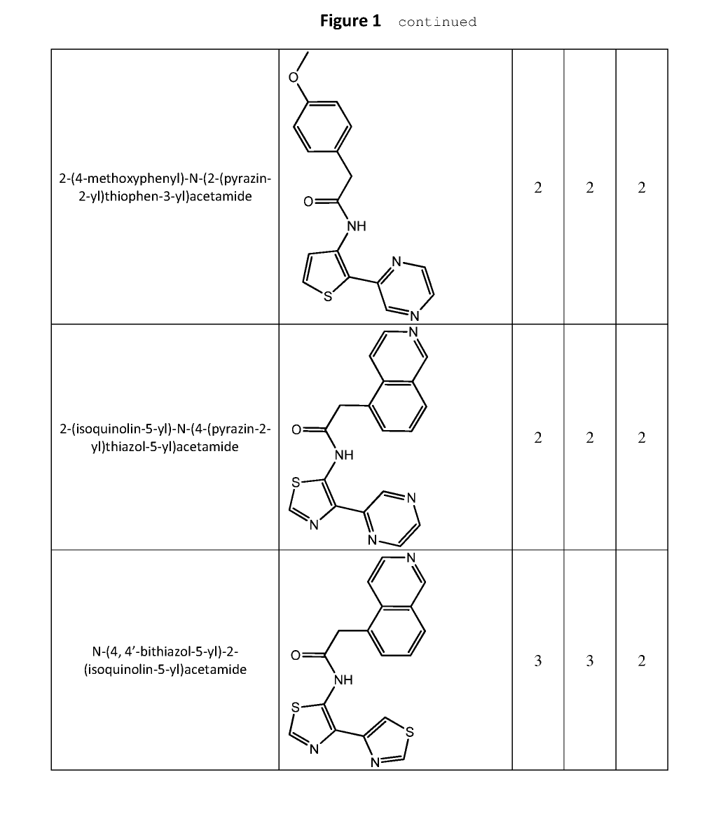

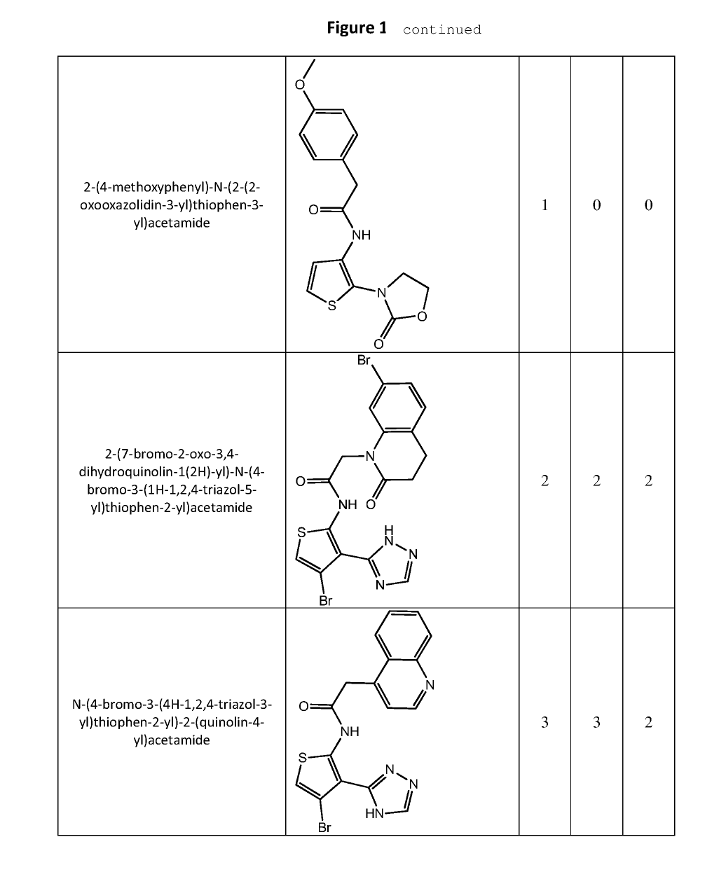

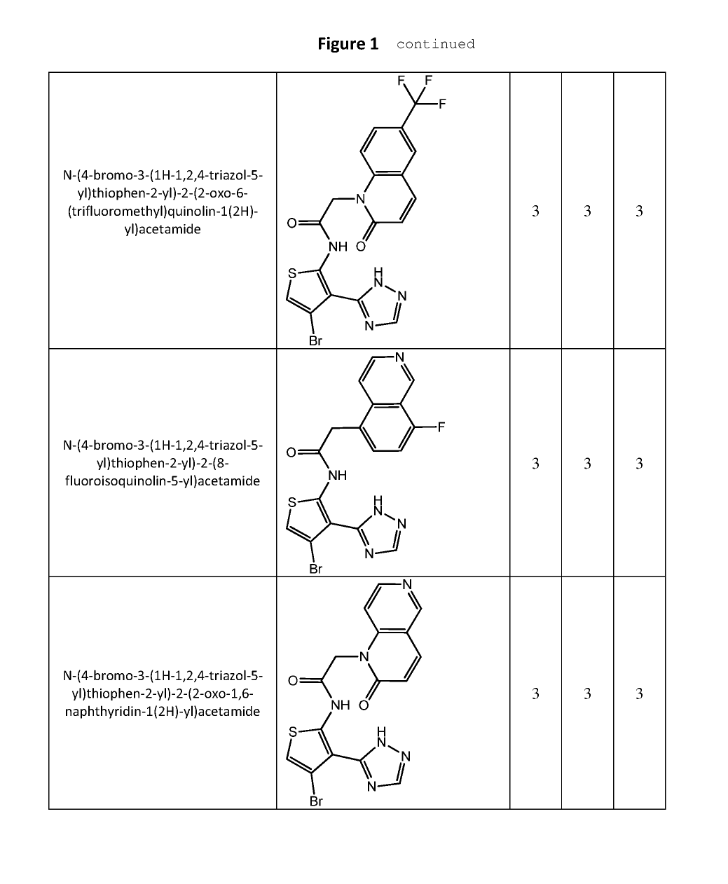

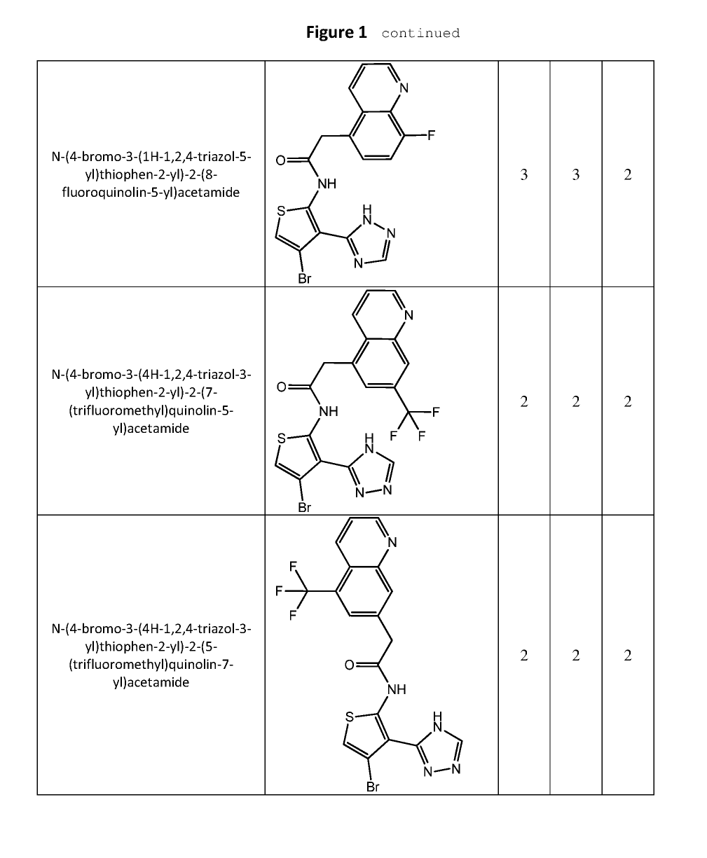

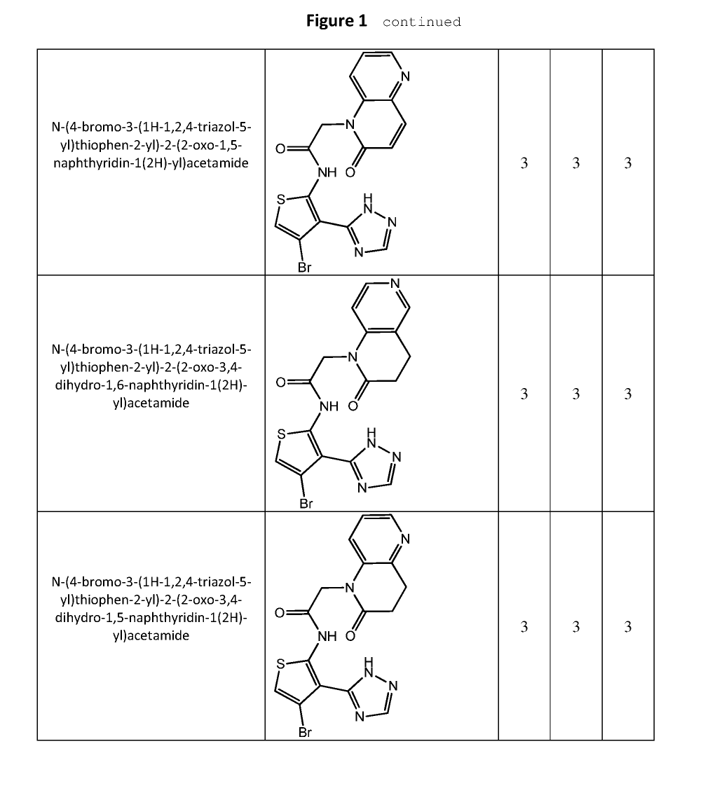

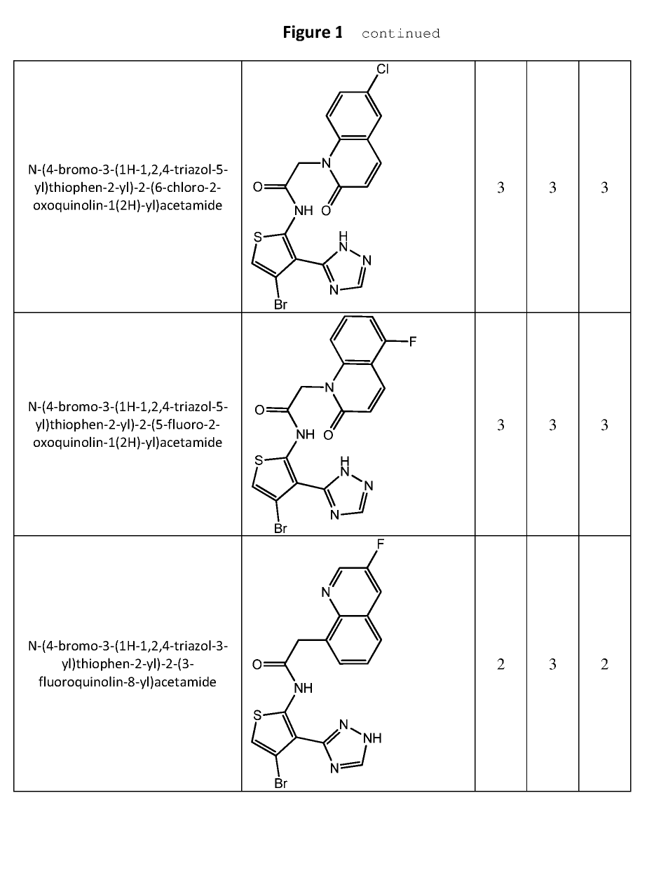

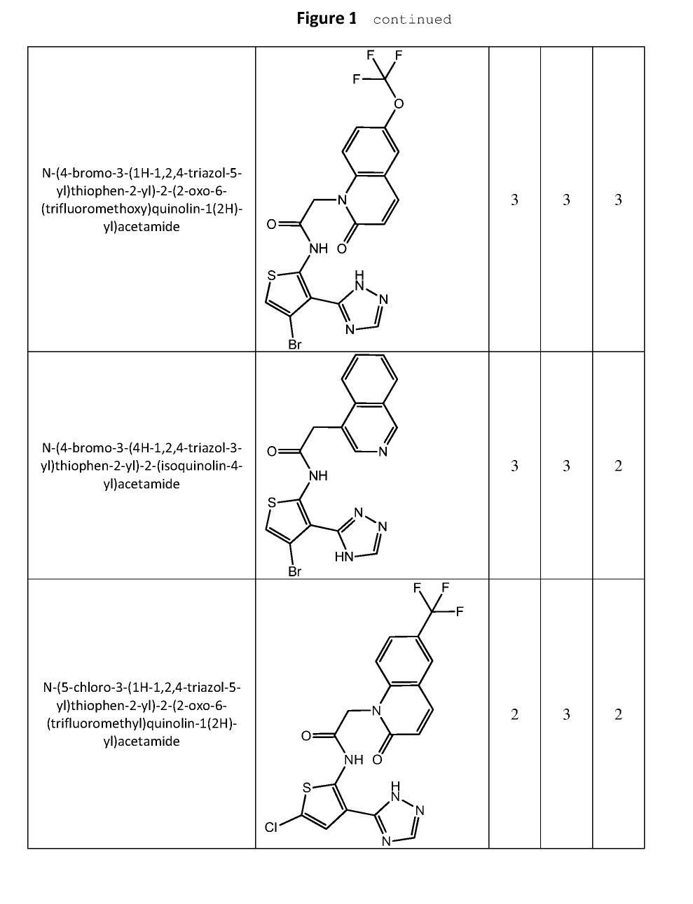

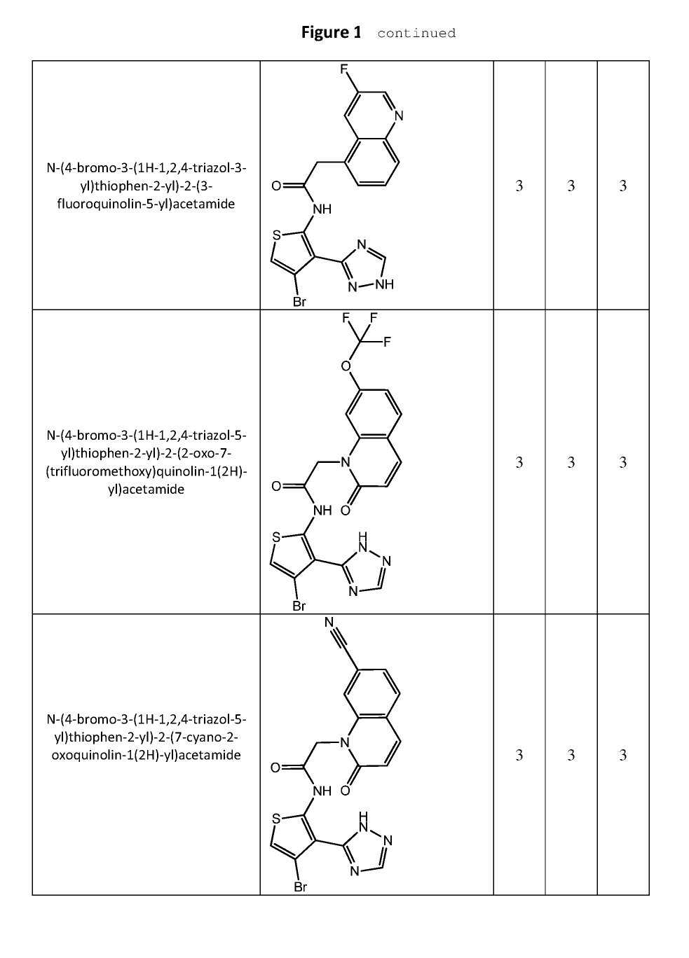

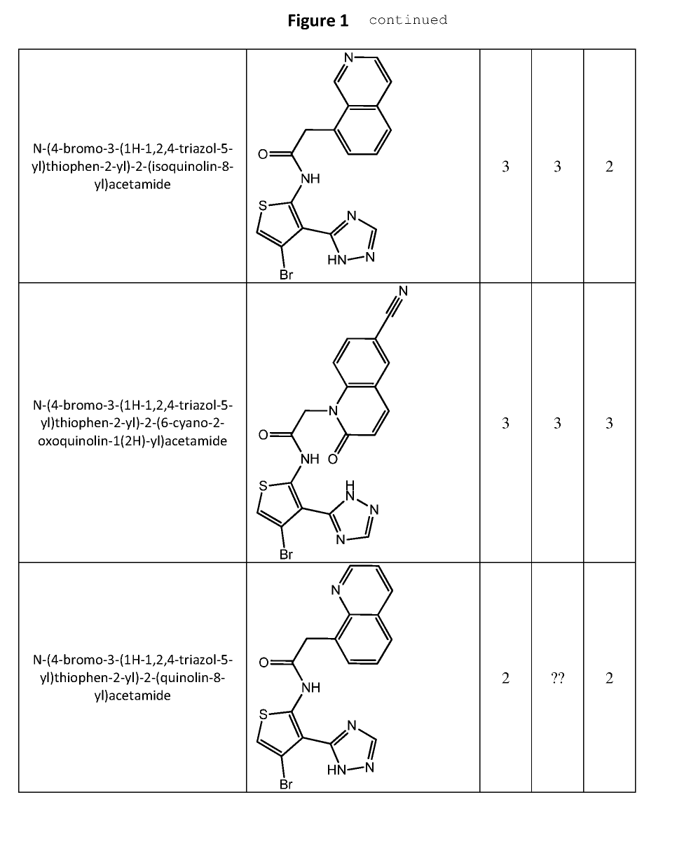

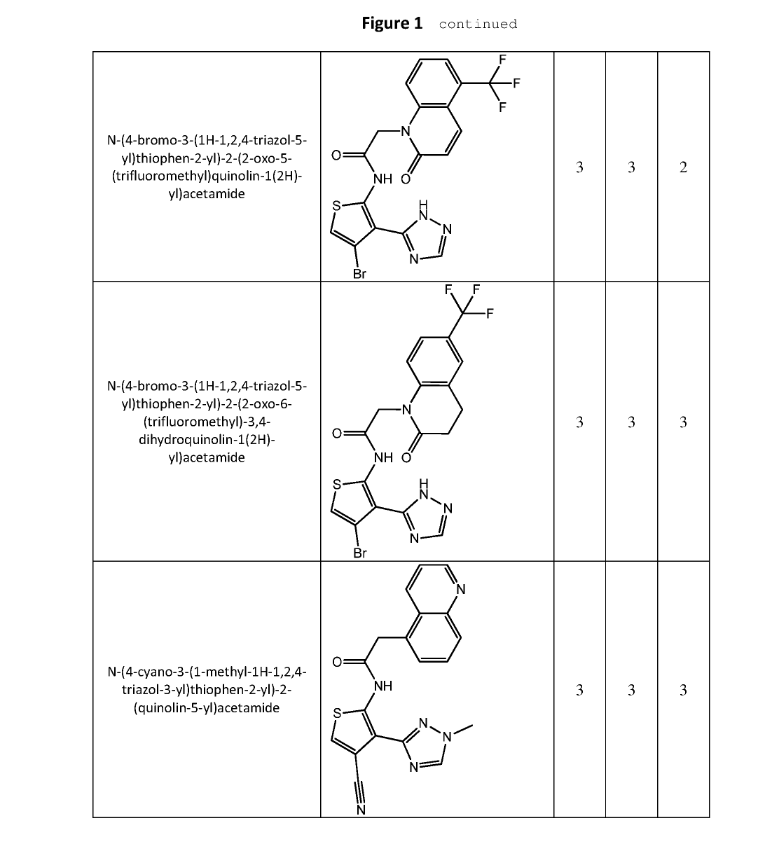

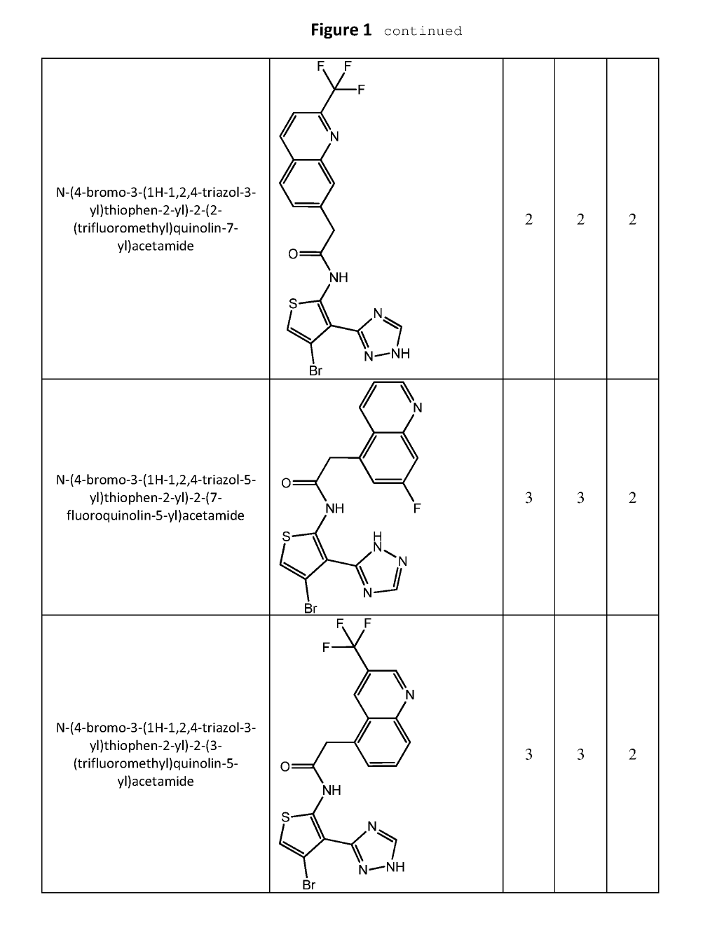

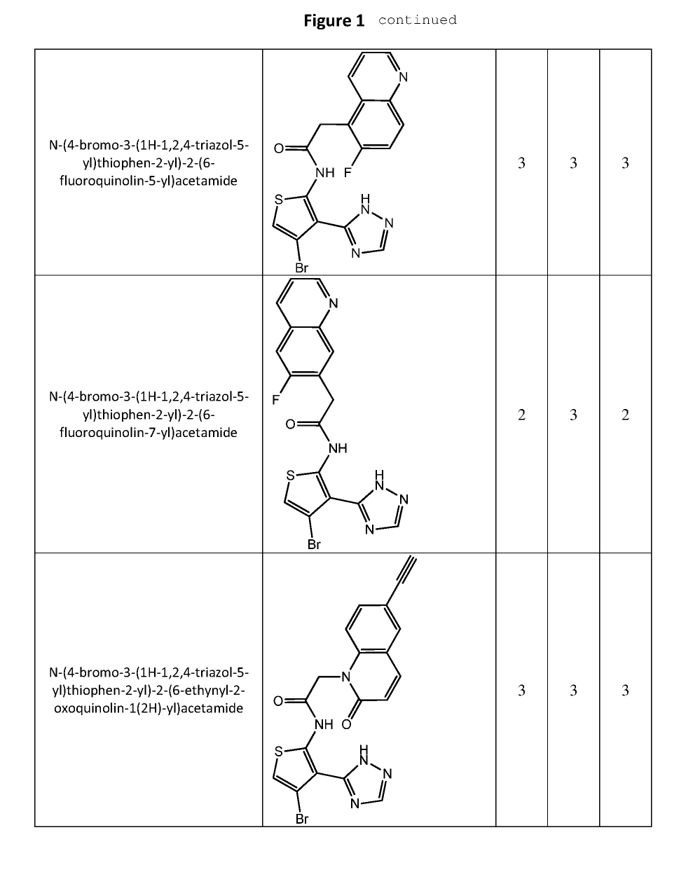

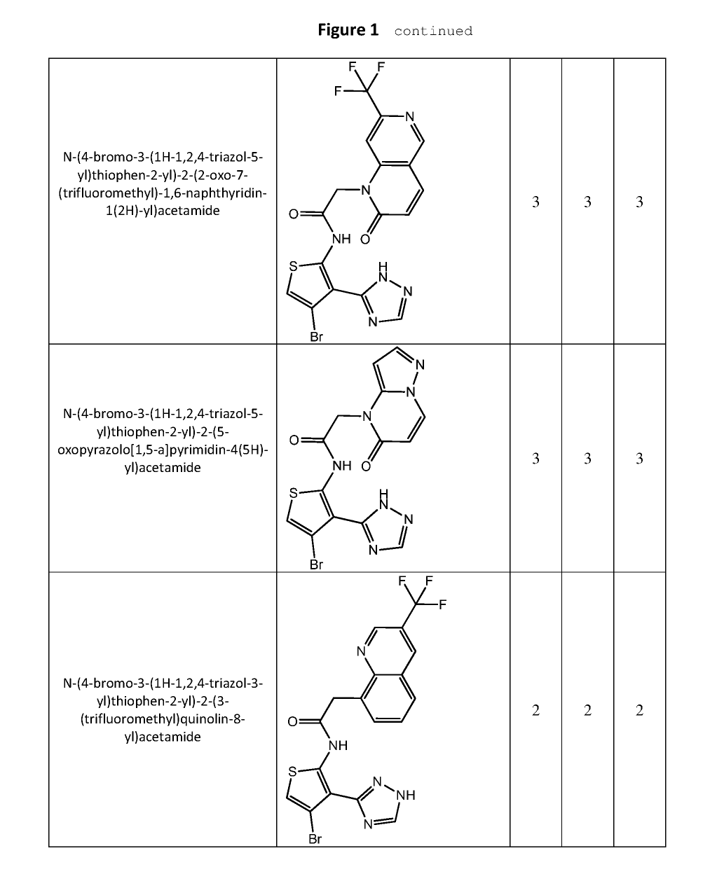

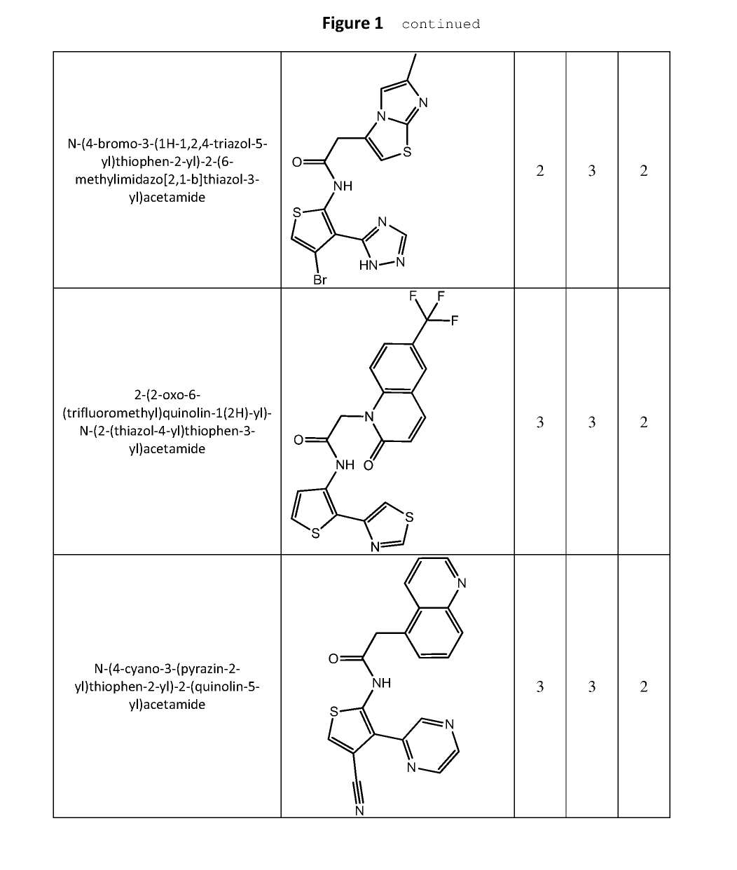

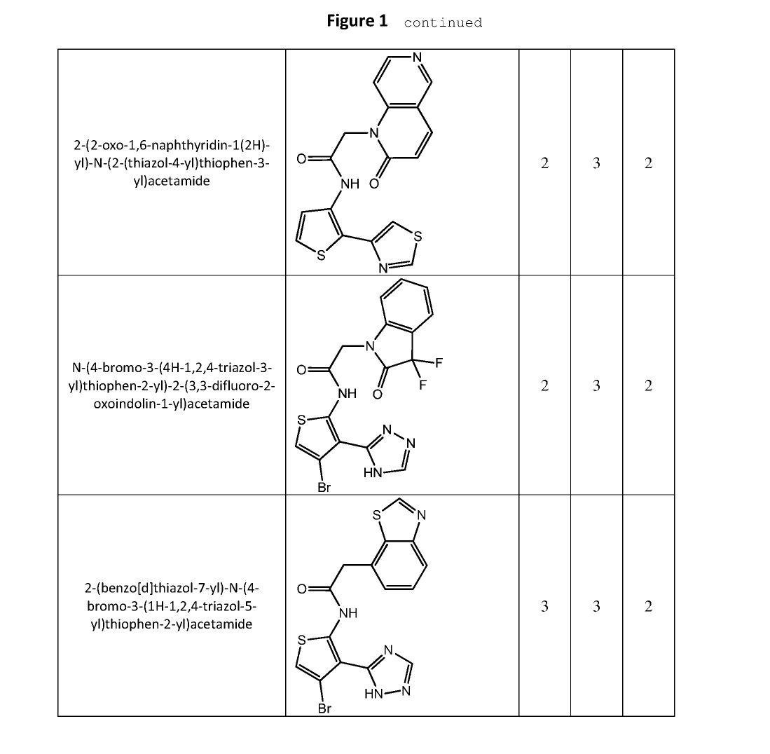

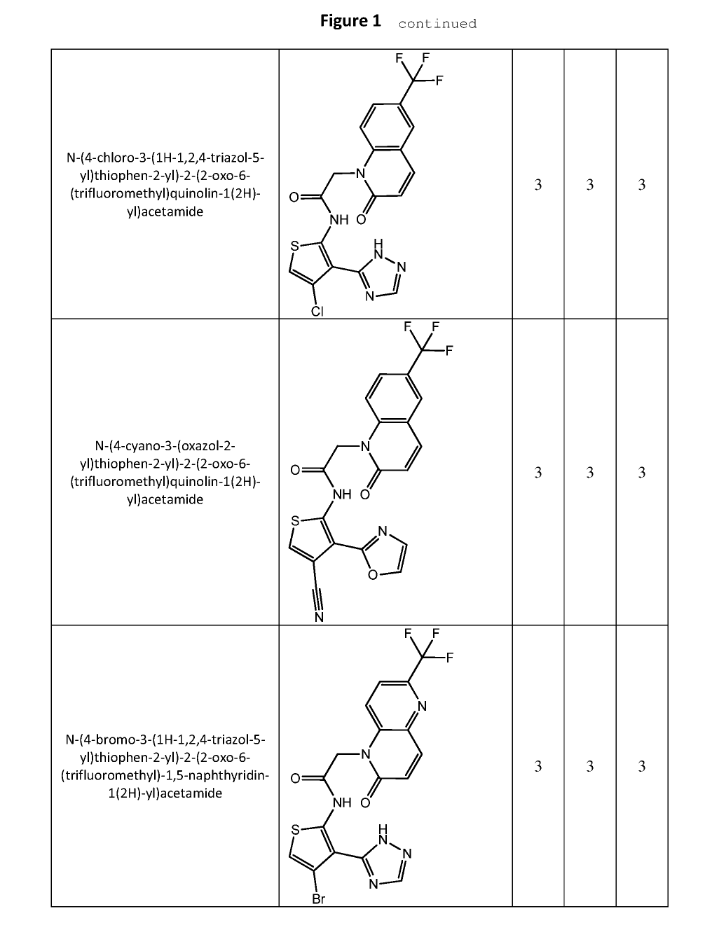

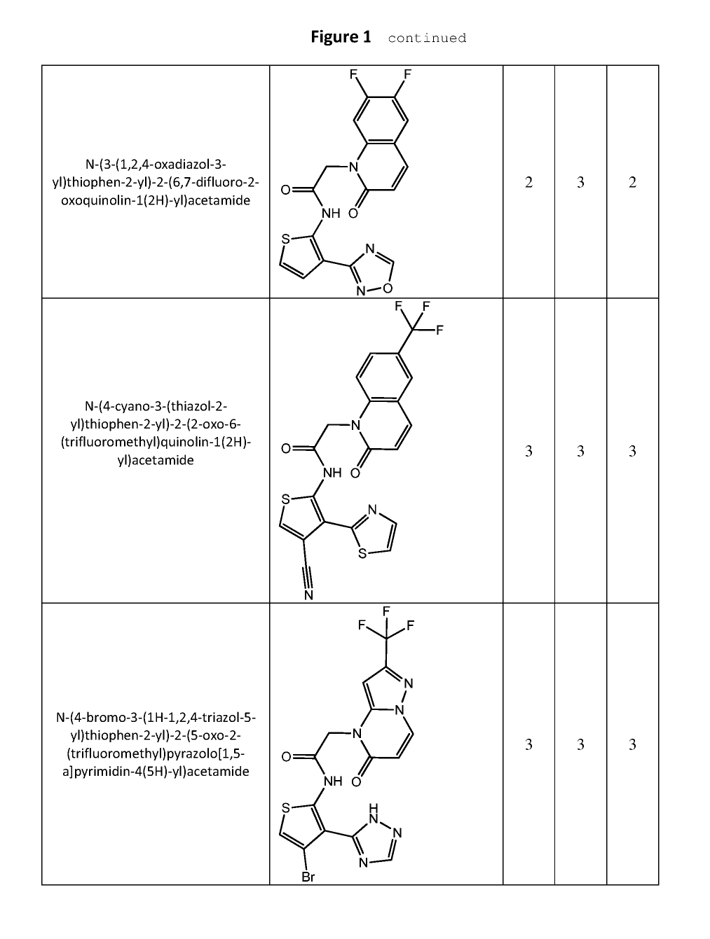

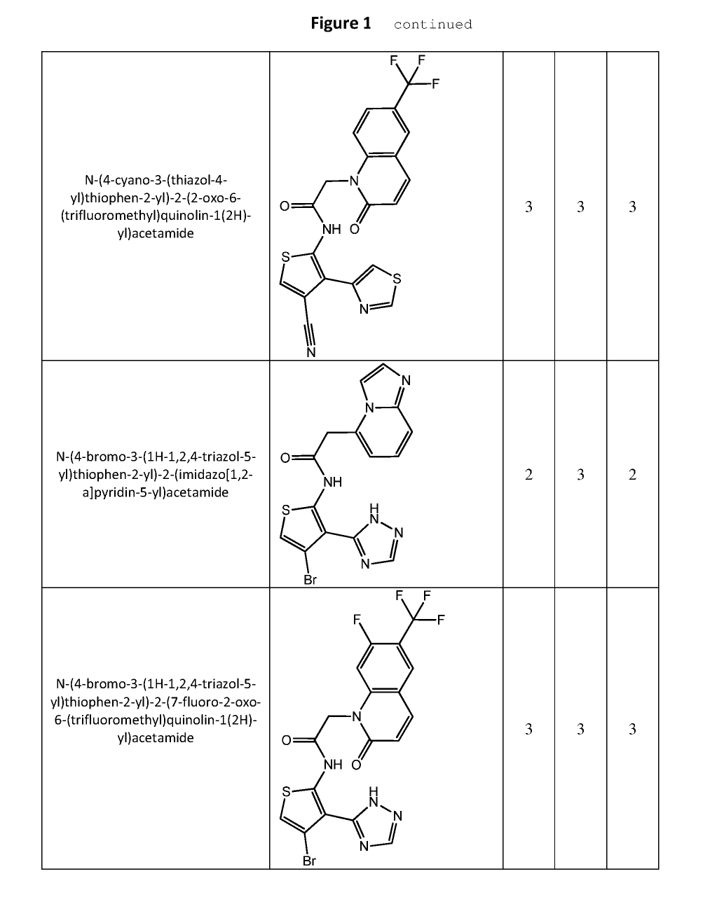

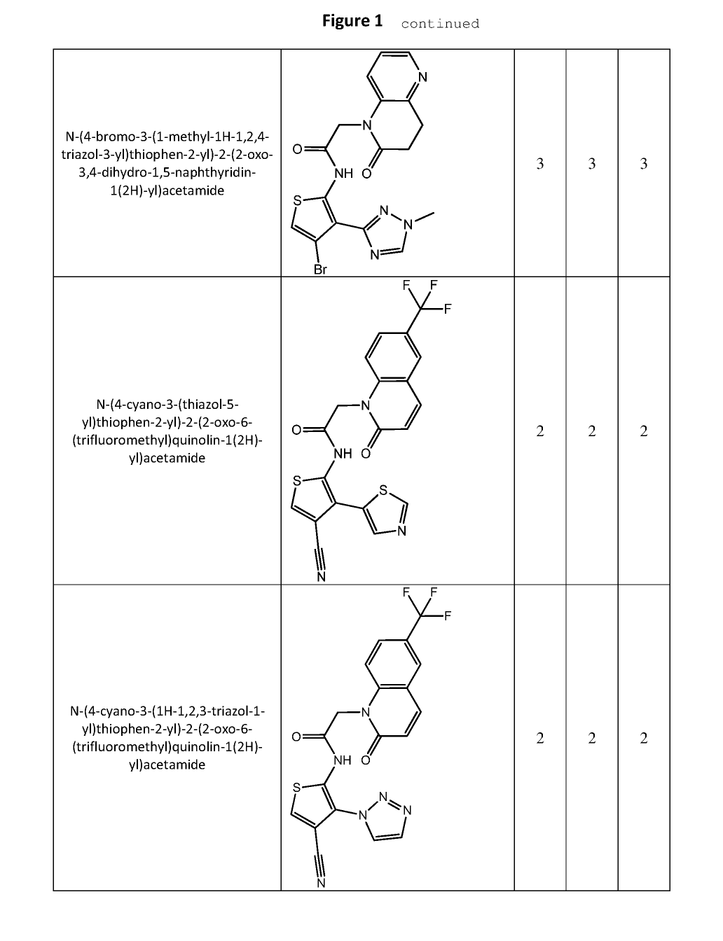

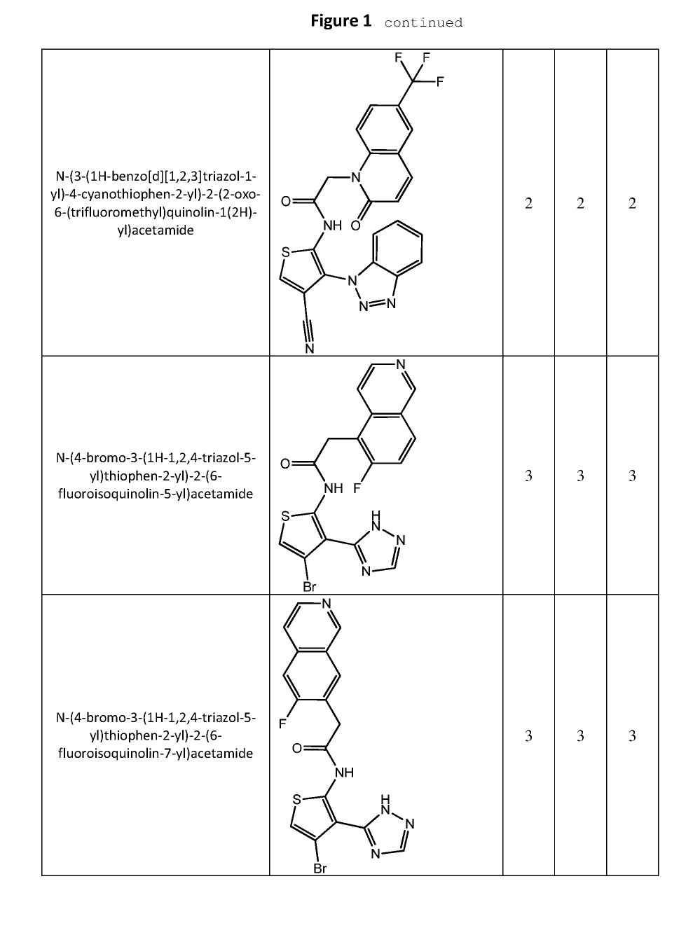

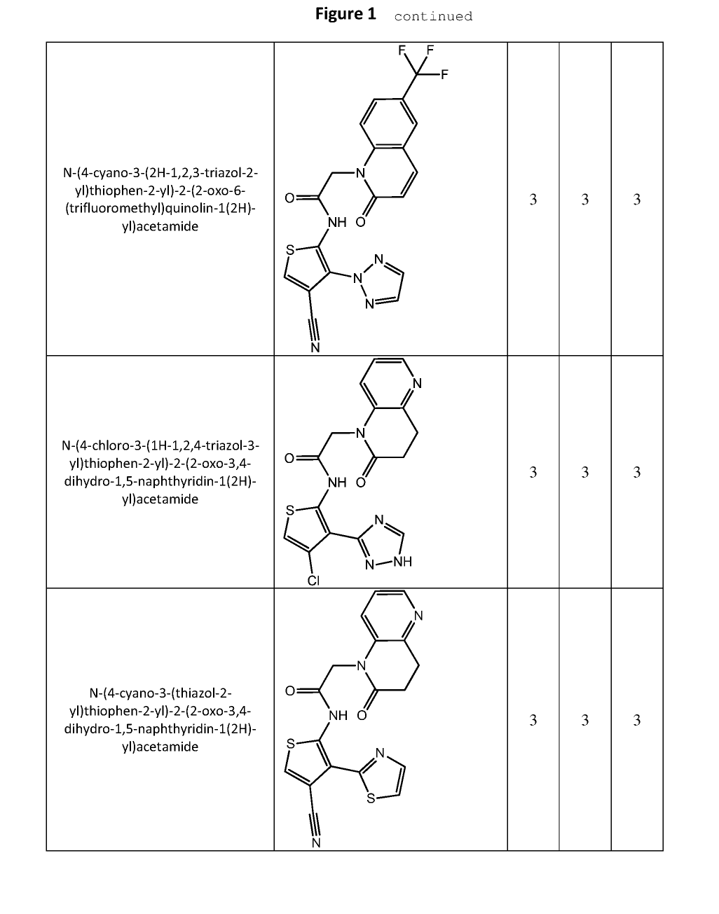

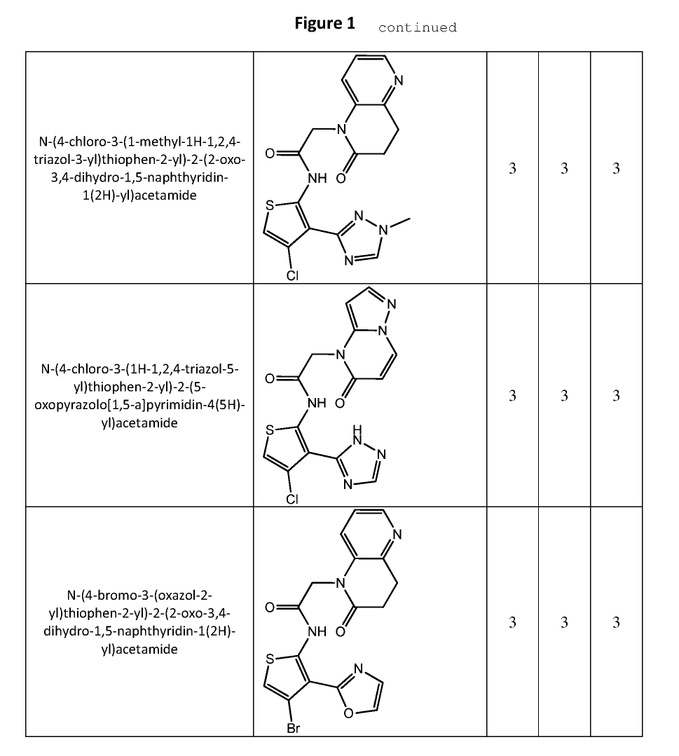

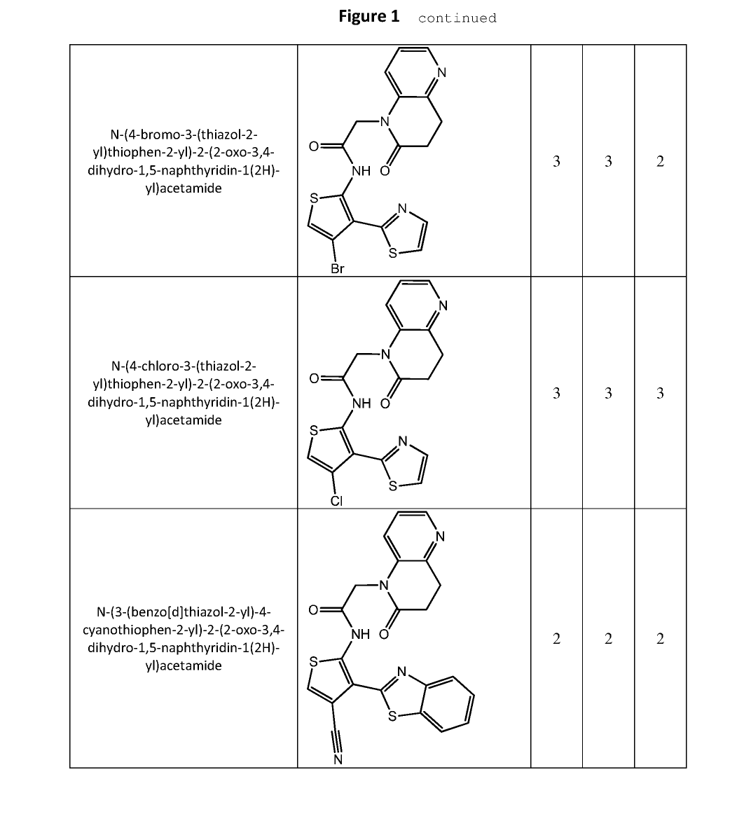

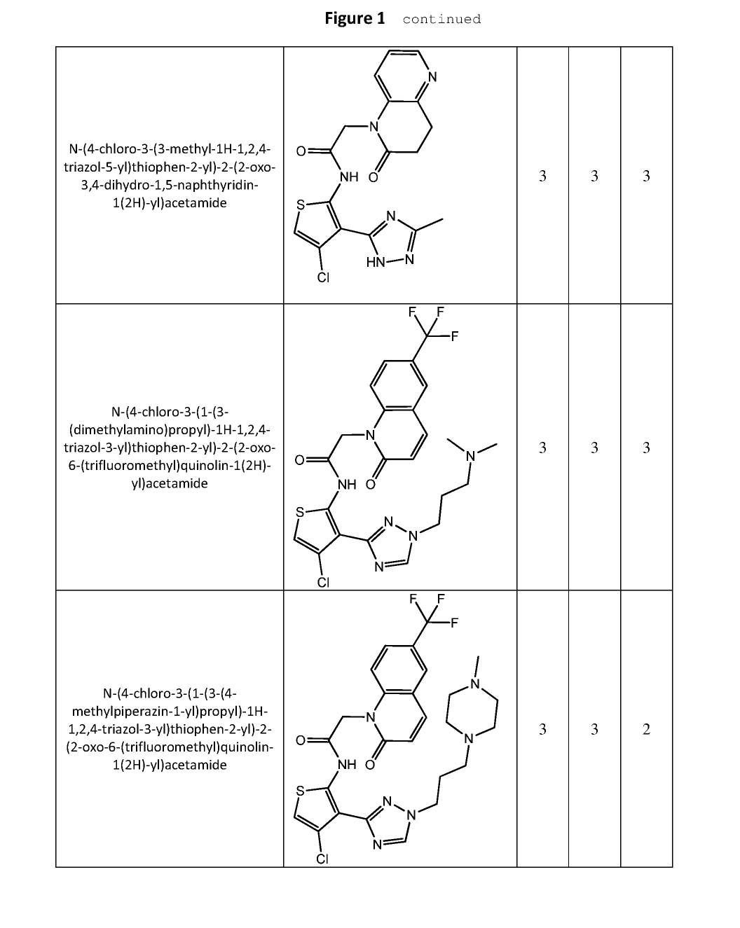

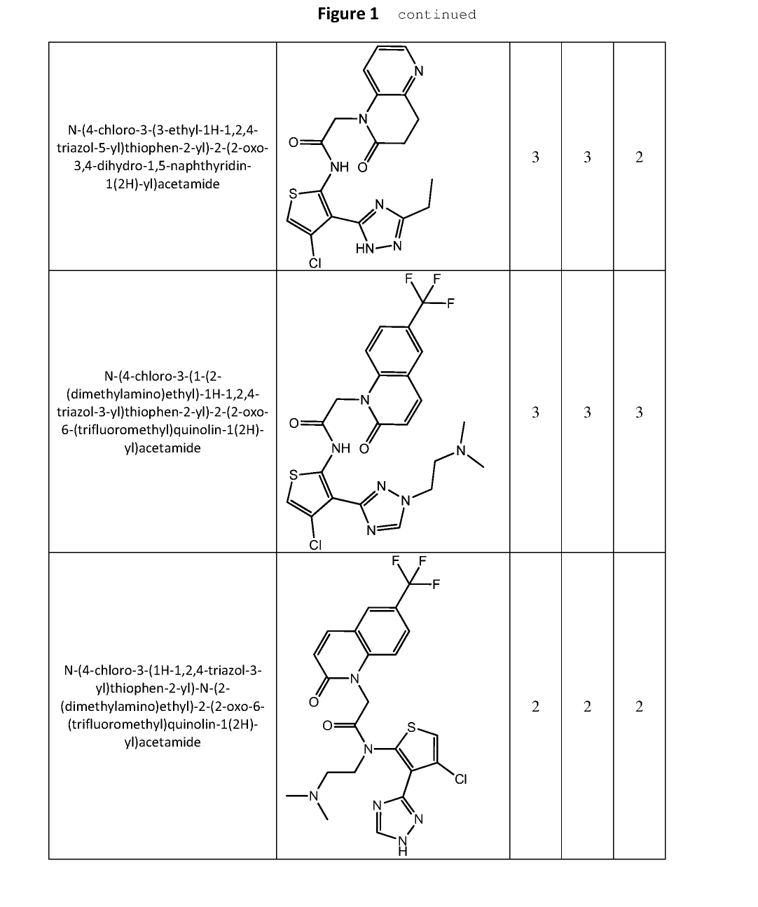

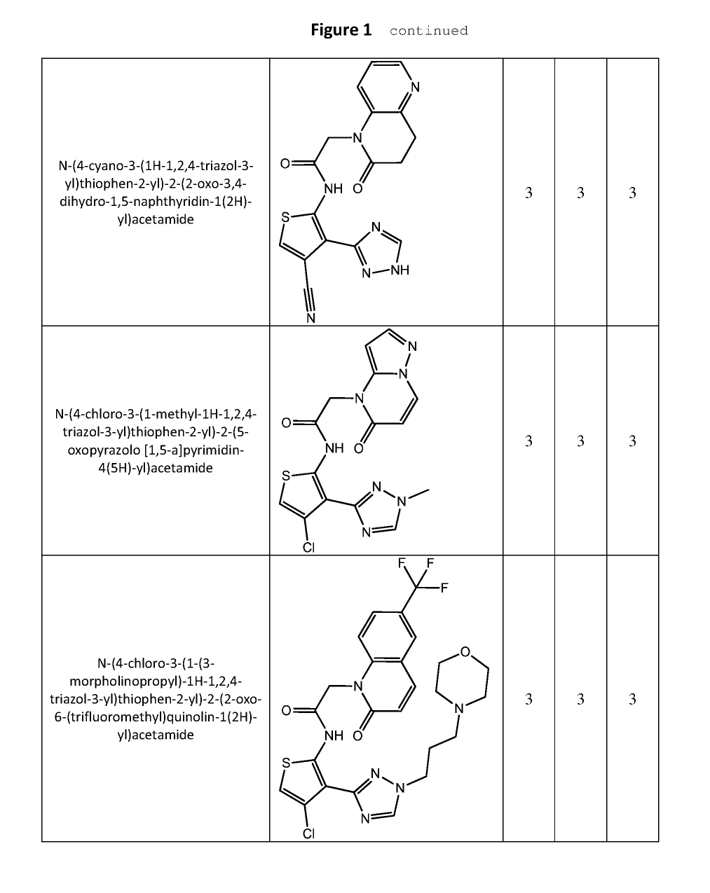

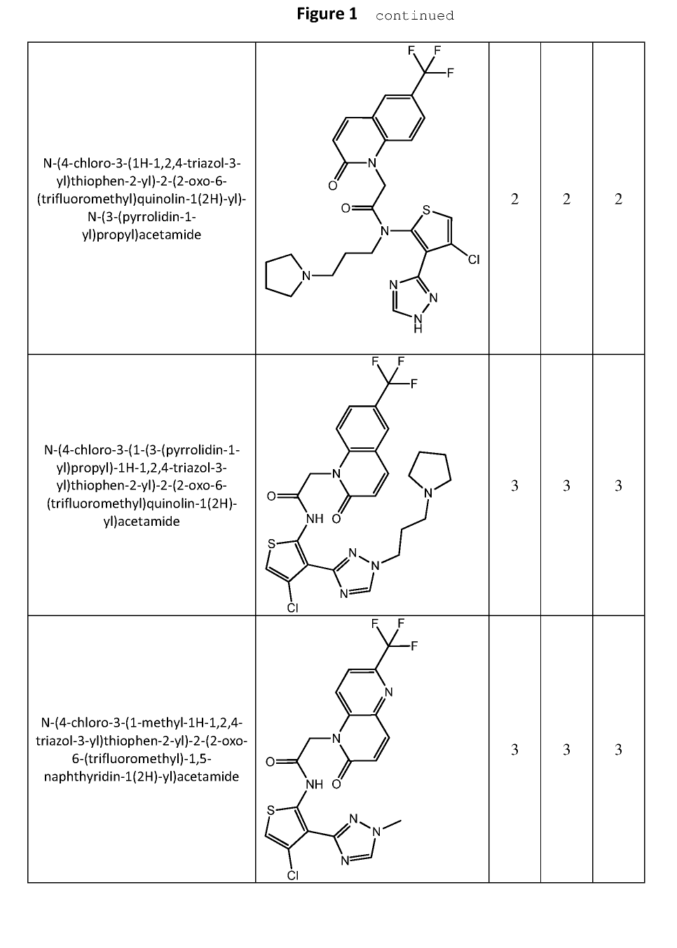

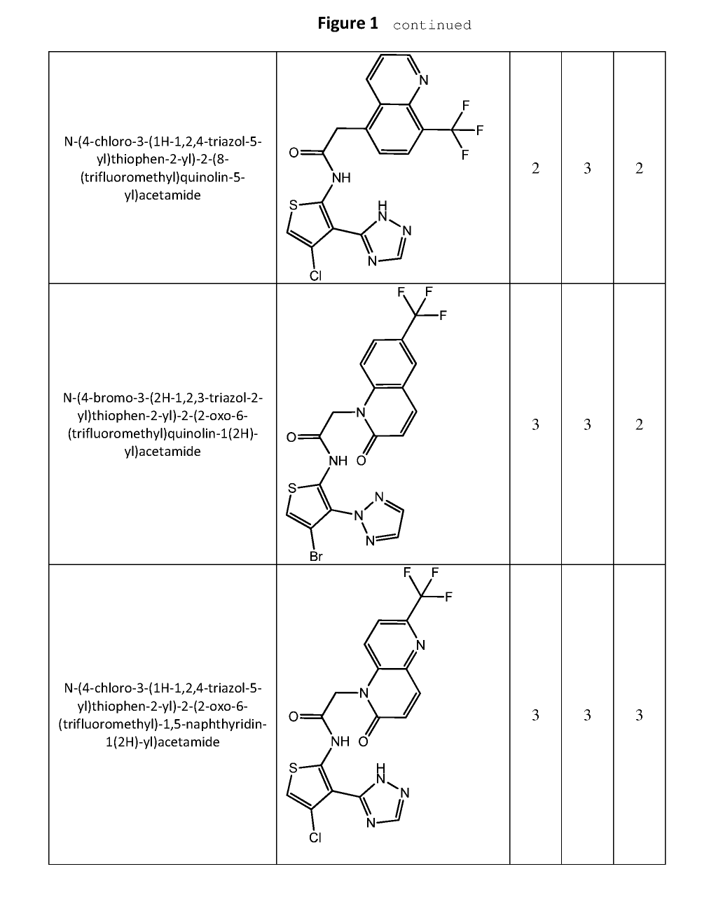

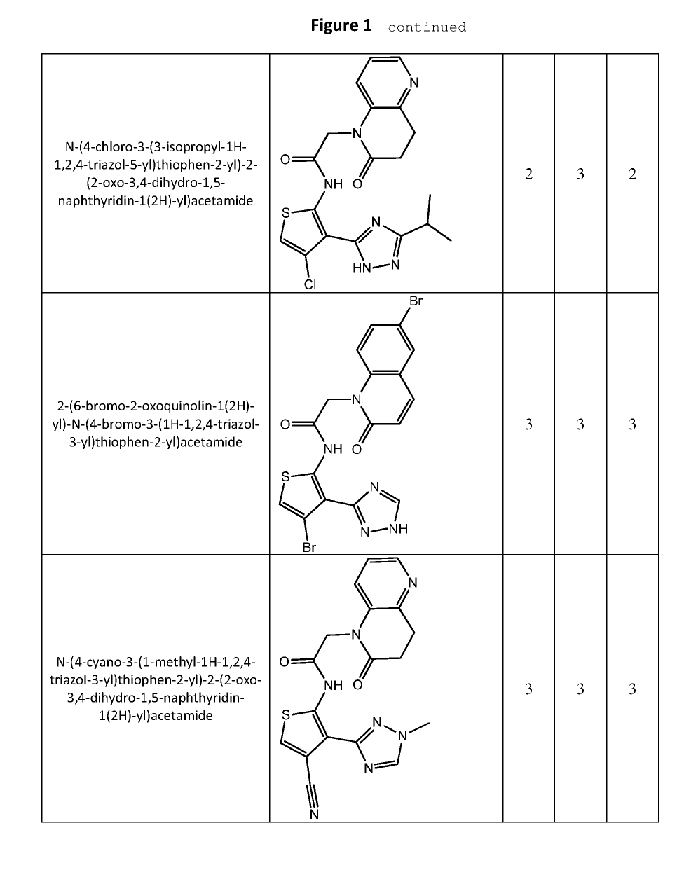

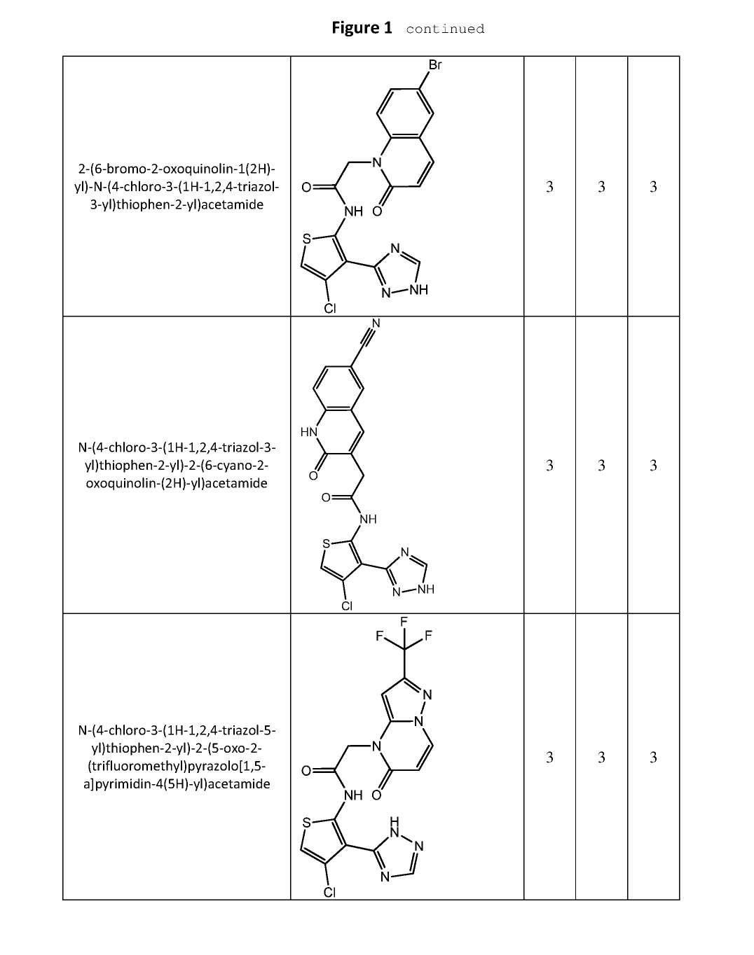

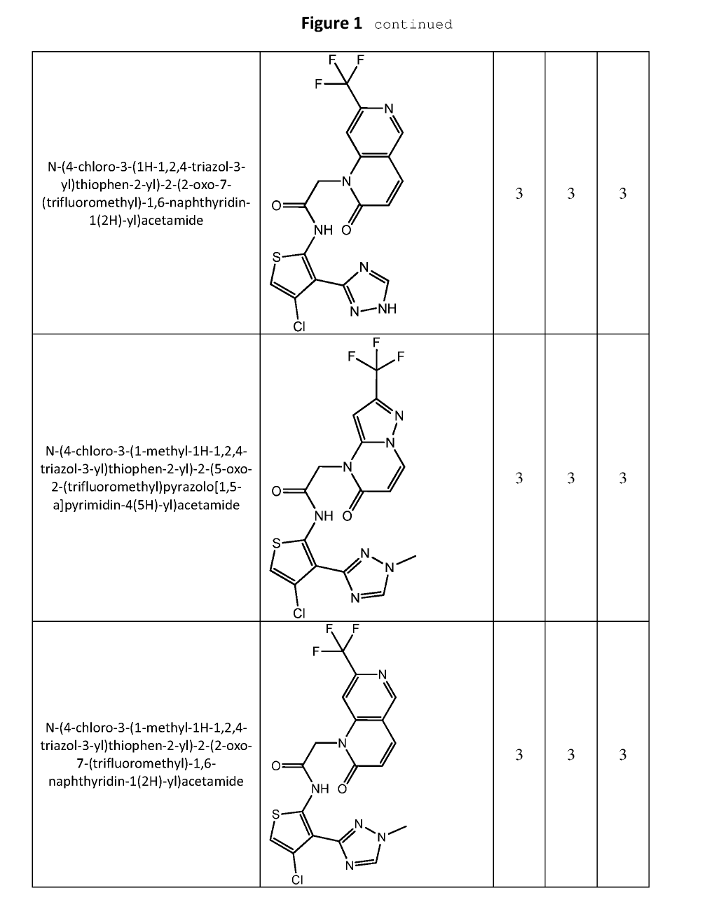

Certain compounds of the present disclosure exhibit various in vitro biological activities as demonstrated, e.g., FIG. 1. For example, certain compounds of the present disclosure exhibit inhibitory activity against Jun N-terminal kinases (JNKs). In vitro assays for the determination of JNK activities are known in the art and exemplary assay formats are described herein. Many compounds of the present disclosure are especially active against JNK3 (e.g., aJNK3 or cJNK3) but may also inhibit JNK1 and JNK2.

In one example, the compounds of the present disclosure may be inhibitors of aJNK3 with an IC50 of less than about 50 μM, less than about 40 μM, less than about 30 μM, less than about 20 μM or less than about 10 μM. In another example, the compounds of the present disclosure may exhibit inhibitory activity against aJNK3 with an IC50 of less than about 9 μM, less than about 8 μM, less than about 7 μM, less than about 6 μM, less than about 5 μM, less than about 4 μM, less than about 3 μM, less than about 2 μM, or less than about 1 μM. In yet another example, the compounds of the present disclosure may exhibit inhibitory activity against aJNK3 with an IC50 of less than about 0.9 μM, less than about 0.8 μM, less than about 0.7 μM, less than about 0.6 μM, less than about 0.5 μM, less than about 0.4 μM, less than about 0.3 μM, less than about

1.2 μM. For example, the compounds of the present disclosure may exhibit inhibitory activity against aJNK3 with an IC50 of less than about 0.1 μM (100 nM). In another example, the compounds of the present disclosure may exhibit inhibitory activity against
a JNK3 with an IC50 of less than about 90 nM, less than about 80 nM, less than about 70 nM, less than about 60 nM, less than about 50 nM, less than about 40 nM, less than about 30 nM or less than about 20 nM. In another example, the compounds of the present disclosure may exhibit inhibitory activity against aJNK3 with an IC50 of less than about lO nM.

Certain compounds of the present disclosure do not only exhibit inhibitory activity against JNK, but at the same time have little or no inhibitory activity against certain other members of the MAP kinase family of proteins. For example, certain compounds of the present disclosure are active against aJNK3 and show little or no inhibitory activity against p38 and/or MAPK. For the purpose of this application the selectivity of the instant compounds for JNK over other kinases is expressed in a ratio of IC50 values. Those can be determined using assays known in the art or those described herein (see e.g., Example 14).

Certain compounds of the present disclosure are characterized by the following inhibitory activities involving aJNK3 and p38. In one example, the ratio of IC50 (aJNK3)/IC50 (p38) is less than about 1, less than about 0.9, less than about 0.8, less than about 0.7, less than about 0.6, less than about 0.5, less than about 0.4, less than about 1.3, less than about 0.2 or less than about 0.1. In another example, the ratio of IC50 (aJNK3)/IC50 (p38) is less than about 0.09, less than about 0.08, less than about 0.07, less than about 0.06, less than about 0.05, less than about 0.04, less than about 0.03, less than about 0.02 or less than about 0.01. In a further example, the ratio of IC₅₀ (aJNK3)/IC₅₀ (p38) is less than about 0.009, less than about 0.008, less than about 0.007, less than about 0.006, less than about 0.005, less than about 0.004, less than about 0.003, less than about 0.002 or less than about 0.001. In yet another example, the ratio of IC₅₀ (aJNK3)/IC₅₀ (p38) is less than about 0.0009, less than about 0.0008, less than about 0.0007, less than about 0.0006, less than about 0.0005, less than about 0.0004, less than about 0.0003, less than about 0.0002 or less than about 0.0001.

Certain compounds of the present disclosure are characterized by the following inhibitory activities involving aJNK3 and MAPK. In one example, the ratio of IC₅₀ (aJNK3)/IC₅₀ (MAPK) is less than about 1, less than about 0.9, less than about 0.8, less than about 0.7, less than about 0.6, less than about 0.5, less than about 0.4, less than about 0.3, less than about 0.2 or less than about 0.1. In another example, the ratio of IC₅₀ (aJNK3)/IC₅₀ (MAPK) is less than about 0.09, less than about 0.08, less than about 0.07, less than about 0.06, less than about 0.05, less than about 0.04, less than about 0.03, less than about 0.02 or less than about 0.01. In a further example, the ratio of IC₅₀ (aJNK3)/IC₅₀ (MAPK) is less than about 0.009, less than about 0.008, less than about 0.007, less than about 0.006, less than about 0.005, less than about 0.004, less than about 0.003, less than about 0.002 or less than about 0.001. In yet another example, the ratio of IC₅₀ (aJNK3)/IC₅₀ (MAPK) is less than about 0.0009, less than about 0.0008, less than about 0.0007, less than about 0.0006, less than about 0.0005, less than about 0.0004, less than about 0.0003, less than about 0.0002 or less than about 0.0001.

Certain compounds of the present disclosure are characterized by the following inhibitory activities involving aJNK3, p38 and MAPK. In one example, the ratio of IC₅₀ (aJNK3)/IC₅₀ (MAPK) and the ratio of IC₅₀ (aJNK3)/IC₅₀ (p38) is each less than about 1, less than about 0.9, less than about 0.8, less than about 0.7, less than about 0.6, less than about 0.5, less than about 0.4, less than about 0.3, less than about 0.2 or less than about 0.1. In another example, the ratio of IC₅₀ (aJNK3)/IC₅₀ (MAPK) and the ratio of IC₅₀ (aJNK3)/IC₅₀ (p38) is each less than about 0.09, less than about 0.08, less than about 0.07, less than about 0.06, less than about 0.05, less than about 0.04, less than about 0.03, less than about 0.02 or less than about 0.01. In a further example, the ratio of IC₅₀ (aJNK3)/IC₅₀ (MAPK) and the ratio of IC₅₀ (aJNK3)/IC₅₀ (p38) is each less than about 0.009, less than about 0.008, less than about 0.007, less than about 0.006, less than about 0.005, less than about 0.004, less than about 0.003, less than about 0.002 or less than about 0.001. In yet another example, the ratio of IC₅₀ (aJNK3)/IC₅₀ (MAPK) and the ratio of IC₅₀ (aJNK3)/IC₅₀ (p38) is each less than about 0.0009, less than about 0.0008, less than about 0.0007, less than about 0.0006, less than about 0.0005, less than about 0.0004, less than about 0.0003, less than about 0.0002 or less than about 0.0001.

Exemplary compounds of the present disclosure and their in vitro biological activities are listed in FIG. 1 and Table 1, below. IC₅₀ values in Table 1 were determined using the procedures as described in U.S. Pat. No. 8,450,353.

TABLE 1
In Vitro Biological Activities

| | JNK3 | JNK1 | JNK2 |
|---|---|---|---|
| Compound Name | IC₅₀ (μm) | IC₅₀ (μm) | IC₅₀ (μm) |
| N-(2-(1H-1,2,4-triazol-5-yl)thiophen-3-yl)- | (++) | (++) | (++) |
| 2-(naphthalen-1-yl)acetamide | | | |
| N-(2-(3-methyl-1H-1,2,4-triazol-5-yl)thiophen-3-yl)-2- | (++) | (++) | (++) |
| (naphthalen-1-yl)acetamide | | | |
| N-(2-(1,3-dimethyl-1H-1,2,4-triazol-5-yl)thiophen-3- | (+) | (+) | (−) |
| yl)-2-(naphthalen-1-yl)acetamide | | | |
| N-(2-(1-methyl-1H-1,2,4-triazol-5-yl)thiophen-3-yl)-2- | (−) | (−) | (−) |
| (naphthalen-1-yl)acetamide | | | |
| 2-(4-methoxyphenyl)-N-(2-(3-methyl-1H-1,2,4-triazol- | (++) | (++) | (++) |
| 5-yl)thiophen-3-yl)acetamide | | | |
| N-(2-(1H-1,2,4-triazol-5-yl)thiophen-3-yl)-2-(4- | (++) | (++) | (++) |
| methoxyphenyl)acetamide | | | |
| N-(2-(1H-1,2,4-triazol-1-yl)thiophen-3-yl)-2-(4- | (+) | (+) | (−) |
| methoxyphenyl)acetamide | | | |
| 2-(4-methoxyphenyl)-N-(4-methyl-3-(3-metyl-1H- | (++) | (++) | (++) |
| 1,2,4-triazol-5-yl)thiophen-2-yl)acetam1de | | | |
| N-(2-(2H-1,2,3-triazol-2-yl)thiophen-3-yl)-2-(4- | (++) | (++) | (++) |
| methoxyphenyl)acetamide | | | |
| N-(2-(3-cyclopropyl-1H-1,2,4-triazol-5-yl)thiophen-3- | (++) | (++) | (++) |
| yl)-2-(4-methoxyphenyl)acetamide | | | |
| N-(2-(3-ethyl-1H-1,2,4-triazol-5-yl)thiophen-3-yl)-2-(4- | (++) | (++) | (++) |
| methoxyphenyl)acetamide | | | |
| N-(2-(3-tert-butyl-1H-1,2,4-triazol-5-yl)thiophen-3- | (++) | (++) | (+) |
| yl)-2-(4-methoxyphenyl)acetamide | | | |
| 2-(4-methoxyphenyl)-N-(2-(3-(tetrahydrofuran 2- | (++) | (++) | (++) |
| yl)-1H-1,2,4-triazol-5-yl)thiophen-3-yl)acetam | | | |
| [symbol] | | | |
| 2-(4-methoxyphenyl)-N-(2-(3-(trifluorometyl)-1H- | (++) | (++) | (+) |
| 1,2,4-triazol-5-yl)thiophen-3-yl)acetam1de | | | |
| N-(4-methyl-3-(1H-1,2,4-triazol-5-yl)thiophen-2yl-2- | (+++) | (+++) | (++)- |
| (2-oxo-3,4-dihydroquinolin-1 (2H)-yl)acetam1de | | | (+++ |
| N-(4-methyl-3-(3-methyl-1H-1,2,4-triazol-5- | (+++) | (+++) | (+++) |
| yl)thiophen-2-yl)-2-(2-oxo-3,4-dihydroquinolin-1 | | | |
| (2H)-yl)acetamide | | | |
| 2-(4-methoxyphenyl)-N-(2-(3-(pyridin-4-yl)-1H-1,2,4- | (+) | (++) | (−) |
| triazol-5-yl)thiophen-3-yl)acetamide | | | |
| N-(2-(3-amino-1H-1,2,4-triazol-5-yl)thiophen-3-yl)-2- | (++) | (++) | (++) |
| (4-methoxyphenyl)acetamide | | | |
| N-(4-chloro-3-(1H-1,2,4-triazol-5-yl)thiophen-2 yl)-2- | (+++) | (+++) | (+++) |
| (2-oxo-3,4-dihydroquinolin-1 (2H)-yl)acetam1de | | | |
| N-(3-(1H-1,2,4-triazol-5-yl)thiophen-2-yl)-2-(2-oxo-3, | (++) | (+++) | (++) |
| 4- | | | |
| dihydroquinolin-1 (2H)-yl)acetamide | | | |
| N-(4-chloro-3-(1H-1,2,4-triazol-5-yl)thiophen-2-yl)-2- | (+++) | (+++) | (+++) |
| (isoquinolin-5-yl)acetamide | | | |
| N-(4-chloro-3-(1H-1,2,4-triazol-5-yl)thiophen-2-yl)- | (+++) | (+++) | (+++) |
| 2-(quinolin-5-yl)acetamide | | | |
| N-(2-(1H-1,2,4-triazol-5-yl)thiophen-3-yl)--(2,3- | (++) | (++) | (++) |
| dihydrobenzo[b] [1,4]dioxin-6-yl)acetam1de | | | |
| N-(4-methyl-3-(1H-1,2,4-triazol-5-yl)thiophen-2-yl)- | (++) | (++) | (++) |
| 2-(quinolin-5-yl)acetamide | | | |
| 2-(2,3-dihydrobenzo[b][1,4]dioxin-6-yl)-N-(2-(3 | (++) | (++) | (++) |
| methyl-1H-1,2,4-triazol-5-yl)thiophen-3-yl)acetam | | | |
| [symbol] | | | |
| N-(2-(1H-1,2,4-triazol-5-yl)thiophen-3-yl)-2- | (++) | (++) | (++) |
| (quinolin-5-yl)acetamide | | | |
| N-(4-methyl-3-(3-methyl-1H-1,2,4-triazol- | (+++) | (+++) | (++) |
| 5-yl)thiophen-2-yl)-2-(quinolin-5- | | | |
| N-(4-methyl-3-(1H-1,2,4-triazol-5-yl)thiophen-2-yl)- | (++) | (++) | (++) |
| 2-(quinoxalin-5-yl)acetamide | | | |
| N-(4-methyl-3-(3-methyl-1H-1,2,4-triazol-5- | (++) | (++) | (++) |
| yl)thiophen-2-yl)-2-(quinoxalin-5- | | | |
| N-(4-methyl-3-(1H-1,2,4-triazol-5-yl)thiophen-2yl)- | (++) | (++) | (++) |
| 2-(4-(3-(piperidin-1-yl)propoxy)phenyl)acetamide | | | |
| N-(4-methyl-3-(3-methyl-1H-1,2,4-triazol-5- | (++) | (++) | (++) |
| yl)thiophen-2-yl)-2-(4-(3-(piperidin- | | | |
| 1-yl)propoxy)phenyl)acetamide | | | |
| 2-(4-(2-(1H-imidazol-1-yl)ethoxy)phenyl)-N-(4-ethyl- | (++) | (++) | (++) |
| 3-(1H-1,2,4-triazol-5-yl)thiophen-2-yl)acetam1de | | | |
| N-(4-bromo-3-(1H-1,2,4-triazol-5-yl)thiophen-2-yl)-2- | (+++) | (+++) | (+++) |
| (isoquinolin-5-yl)acetamide | | | |
| N-(4-bromo-3-(1H-1,2,4-triazol-5-yl)thiophen-2-yl)-2- | (+++) | (+++) | (+++) |
| (quinolin-5-yl)acetamide | | | |
| N-(4-bromo-3-(1H-1,2,4-triazol-5-yl)thiophen-2-yb1)-2- | (+++) | (+++) | (+++) |
| (2-oxo-3,4-dihydroquinolin-1 (2H)-yl)acetam1de | | | |
| N-(4-cyano-3-(1H-1,2,4-triazol-5-yl)thiophen-2-yl)-2- | (+++) | (+++) | (+++) |
| (isoquinolin-5-yl)acetamide | | | |
| N-(4-cyano-3-(1H-1,2,4-triazol-5-yl)thiophen-2y-1)-2- | (+++) | (+++) | (+++) |
| (2-oxo-3,4-dihydroquinolin-1 (2H)-yl)acetam1de | | | |
| N-(2-(3-methyl-1H-1,2,4-triazol-5-yl)thiophe-3-yl)-2- | (++) | (++) | (++) |
| (4-(2-oxopyrrolidin-1-yl)phenyl)acetamide | | | |
| N-(4-methyl-3-(5-methyl-4H-1,2,4-triazol-3- | (++) | (++) | (++) |
| yl)thiophen-2-yl)-2-(4-(pyridin-4-yl)phenyl)acetamide | | | |
| N-(4-cyano-3-(4H-1,2,4-triazol-3-yl)thiophen-2-yl)- | (+++) | (+++) | (+++) |
| 2-(quinolin-5-yl)acetamide | | | |
| N-(4-bromo-3-(1H-1,2,4-triazol-5-yl)thiophen-2-yl) | (+++) | (+++) | (+++) |
| 2-(2-oxo-7-(trifluoromethyl)quinolin-1 (2H)- | | | |
| N-(4-bromo-3-(1H-1,2,4-triazol-5-yl)thiophen-2-yl)- | (+++) | (+++) | (+++) |
| 2-(6-fluoro-2-oxo-3,4-dihydroquinolin-1 (2H)- | | | |
| yl)acetamide | | | |
| N-(4-bromo-3-(1H-1,2,4-triazol-5-yl)thiophen-2-yl)- | (+++) | (+++) | (+++) |
| 2-(7-fluoro-2-oxoquinolin-1 (2H)-yl)acetam1de | | | |
| N-(4-bromo-3-(1H-1,2,4-triazol-5-yl)thiophen-2-yl)- | (+++) | (+++) | (+++) |
| 2-(7-chloro-2-oxo-3,4-dihydroquinolin-1 (2H)- | | | |
| yl)acetamide | | | |
| N-(4-bromo-3-(1H-1,2,4-triazol-5-yl)thiophen-2y-1)- | (+++) | (+++) | (+++) |
| 2-(6,7-difluoro-2-oxoquinolin-1 (2H)- | | | |
| N-(4-bromo-3-(1H-1,2,4-triazol-5-yl)thiophen-2-yl) | (+++) | (+++) | (+++) |
| 2-(2-oxo-6-(trifluoromethyl)quinolin-1 (2H)- | | | |
| N-(4-bromo-3-(1H-1,2,4-triazol-5-yl)thiophen-2-yl)- | (+++) | (+++) | (+++) |
| 2-(6-fluoro-2-oxoquinolin-1 (2H)-yl)acetam1de | | | |
| 2-(isoquinolin-5-yl)-N-(2-(4-methylthiazol- | (++) | (++) | (++) |
| 2-yl)thiophen-3-yl)acetamide | | | |
| 2-(isoquinolin-5-yl)-N-(2-(thiazol-4-yl)thiophen- | (++) | (++) | (++) |
| 3-yl)acetamide | | | |
| 2-(2-oxo-3,4-dihydro-1,6-naphthyridin-1 (2H)-yl)-N- | (++) | (++) | (++) |
| (2-(thiazol-4-yl)thiophen-3-yl)acetamide | | | |
| 2-(2-oxo-3,4-dihydroquinolin-1 (2H)-yl)-N-(2-(thiazol-4- | (++) | (++) | (++) |
| yl)thiophen-3-yl)acetamide | | | |
| 2-(isoquinolin-5-yl)-N-(2-(2-methoxythiazol-4- | (++) | (++) | (+) |
| yl)thiophen-3-yl)acetamide | | | |
| N-(2-(2-chlorothiazol-4-yl)thiophen-3-yl)-2- | (++) | (++) | (+) |
| (isoquinolin-5-yl)acetamide | | | |
| 2-(isoquinolin-5-yl)-N-(2-(thiazol-2-yl)thiophen-3- | (++) | (++) | (++) |
| yl)acetamide | | | |
| 2-(isoquinolin-5-yl)-N-(2-(5-methylthiazol-2- | (++) | (++) | (++) |
| yl)thiophen-3-yl)acetamide | | | |
| 2-(4-(3-(piperidin-1-yl)propoxy)phenyl)-N-(2-(thiazol- | (++) | (++) | (++) |
| 4-yl)thiophen-3-yl)acetamide | | | |
| N-(3-(benzo[d]thiazol-2-yl)-4-methylthiophen-2-yl)-2- | (++) | (++) | (+) |
| (isoquinolin-5-yl)acetamide | | | |
| 2-(4-methoxyphenyl)-N-(2-(oxazol-2-yl)thiophen- | (++) | (++) | (+) |
| 3-yl)acetamide | | | |
| 2-(isoquinolin-5-yl)-N-(2-(oxazol-2-yl)thiophen- | (++) | (++) | (++) |
| 3-yl)acetamide | | | |
| 2-(4-methoxyphenyl)-N-(3-(5-methyl-1,2,4-oxadiazol- | (++) | (++) | (++) |
| 3-yl)thiophen-2-yl)acetamide | | | |
| N-(2-(1,3,4-oxadiazol-2-yl)thiophen-3-yl)- | (++) | (++) | (+) |
| 2-(naphthalen-1-yl)acetamide | | | |
| 2-(4-methoxyphenyl)-N-(2-(5-methyl-1,3,4-oxadiazol- | (++) | (++) | (−) |
| 2-yl)thiophen-3-yl)acetamide | | | |
| N-(2-(5-isopropyl-1,3,4-oxadiazol-2-yl)thiophen-3- | (+) | (+) | (−) |
| yl)-2-(4-methoxyphenyl)acetamide | | | |
| N-(2-(5-methyl-1,3,4-oxadiazol-2-yl)thiophen-3-yl)- | (++) | (++) | (+) |
| 2-(naphthalen-1-yl)acetamide | | | |
| N-(4-methyl-3-(3-methyl-1,2,4-oxadiazol-5- | (++) | (++) | (++) |
| yl)thiophen-2-yl)-2-(naphthalen-1- | | | |
| N-(4-methyl-3-(3-methyl-1,2,4-oxadiazol-5-yl | (++) | (++) | (++) |
| thiophen-2-yl)-2-(4-(pyridin-4- | | | |
| N-(2-(3-methyl-1,2,4-oxadiazol-5-yl)thiophen-3-yl)- | (++) | (++) | (−) |
| 2-(naphthalen-1-yl)acetamide | | | |
| N-(4-(1H-1,2,4-triazol-5-yl)thiazol-5-yl)-2- | (++) | (++) | (++) |
| (isoquinolin-5-yl)acetamide | | | |
| 2-(isoquinolin-5-yl)-N-(4-(1-methyl-1H-1,2,4-triazol-5- | (++) | (++) | (++) |
| yl)thiazol-5-yl)acetamide | | | |
| 2-(2-pyridyl)-3-(1-naphthylacetylamino)thiophene | (++) | | |
| N-(2-(1H-pyrazol-1-yl)thiophen-3-yl)-2-(4- | (+) | (+) | (−) |
| methoxyphenyl)-acetamide | | | |
| 2-(4-methoxyphenyl)-N-(2-(4-methyl-1H-pyrazol-1- | (+) | (+) | (+) |
| yl)thiophen-3-yl)acetamide | | | |
| N-(2-(1H-pyrazol-3-yl)thiophen-3-yl)-2-(naphthalen-1- | (++) | (++) | (−) |
| yl)acetamide | | | |
| N-(2-(1-methyl-1H-pyrazol-3-yl)thiophen-3-yl)-2- | (+) | (+) | (−) |
| (naphthalen-1-yl)acetamide | | | |
| N-(2-(5-methyl-1H-pyrazol-3-yl)thiophen-3-yl)-2- | (++) | (++) | (−) |
| (naphthalen-1-yl)acetamide | | | |
| N-(3-(2H-tetrazol-5-yl)thiophen-2-yl)-2-(4- | (++) | (++) | (+) |
| methoxyphenyl)-acetamide | | | |
| 2-(4-methoxyphenyl)-N-(3-(2-methyl-2H-tetrazol- | (++) | (++) | (+) |
| 5-yl)thiophen-2-yl)acetamide | | | |
| N-(3-(2-(methoxymethyl)-2H-tetrazol-5-yl)thiophen- | (−) | (−) | (−) |
| 2-yl)-2-(4-methoxyphenyl)acetamide | | | |
| N-(3-(1-(methoxymethyl)-1H-tetrazol-5-yl)thiophen-2- | (++) | (++) | (−) |
| yl)-2-(4-methoxyphenyl)acetamide | | | |
| N-(2-(1-methyl-1H-imidazol-2-yl)thiophen-3-yl)- | (+) | (+) | (−) |
| 2-(naphthalen-1-yl)acetamide | | | |
| 2-(4-methoxyphenyl)-N-(2-(1-methyl-1H-imidazol- | (+) | (+) | (−) |
| 4-yl)thiophen-3-yl)acetamide | | | |
| N-(2-(1H-imidazol-4-yl)thiophen-3-yl)-2- | (+) | (+) | (−) |
| (4-methoxyphenyl)-acetamide | | | |
| N-(2-(1H-imidazol-4-yl)thiophen-3-yl)-2-(2-oxo- | (++) | (++) | (+) |
| 3,4-dihydroquinolin-1 (2H)-yl)acetamide | | | |
| 2-(4-methoxyphenyl)-N-(2-(2-methyl-1H-imidazol- | (+) | (+) | (−) |
| 4-yl)thiophen-3-yl)acetamide | | | |
| N-(2-(2-methyl-1H-imidazol-4-yl)thiophen-3-yl)-2- | (+) | (+) | (−) |
| (2-oxo-3,4-dihydroquinolin-1 (2H)- | | | |
| N-(2-(1H-imidazol-1-yl)thiophen-3-yl)2-(naphthalen- | (++) | (+) | (−) |
| 1-yl)acetamide | | | |
| 2-(4-methoxyphenyl)-N-(2-(4-methyl-1H-imidazol-1- | (−) | (−) | (−) |
| yl)thiophen-3-yl)acetamide | | | |
| 2-(4-methoxyphenyl)-N-(2-(pyrazin-2-yl)thiophen- | (++) | (++) | (++) |
| 3-yl)acetamide | | | |
| 2-(isoquinolin-5-yl)-N-(4-(pyrazin-2-yl)thiazol- | (++) | (++) | (++) |
| 5-yl)acetamide | | | |
| N-(4,4′-bithiazol-5-yl)-2-(isoquinolin-5-yl)acetamide | (+++) | (+++) | (++) |
| 2-(4-methoxyphenyl)-N-(2-(2-oxooxazolidin-3- | (+) | (−) | (−) |
| yl)thiophen-3-yl)acetamide | | | |
| 2-(7-bromo-2-oxo-3,4-dihydroquinolin-1(2H)-yl)-N-(4- | (++) | (++) | (++) |
| bromo-3-(1H-1,2,4-triazol-5-yl)thiophen-2- | | | |
| N-(4-brornlo-3-(4H-1,2,4-triazol-3-yl)thiophen-2-yl)-2- | (+++) | (+++) | (++) |
| (quinolin-4-yl)acetamide | | | |
| N-(4-bromo-3-(1H-1,2,4-triazol-5-yl)thiophen-2-yl) 2- | (+++) | (+++) | (+++) |
| (2-oxo-6-(trifluoromethyl)quinolin-1 (2H)- | | | |
| [symbol] | | | |
| N-(4-bromo-3-(1H-1,2,4-triazol-5-yl)thiophen-2-yl)-2- | (+++) | (+++) | (+++) |
| (8-fluoroisoquinolin-5-yl)acetamide | | | |
| N-(4-bromo-3-(1H-1,2,4-triazol-5-yl)thiophen-2-yl)- | (+++) | (+++) | (+++) |
| 2-(2-oxo-1,6-naphthyridin-1 (2H)-yl)acetamide | | | |
| N-(4-bromo-3-(1H-1,2,4-triazol-5-yl)thiophen-2-yl)- | (+++) | (+++) | (++) |
| 2-(8-fluoroquinolin-5-yl)acetamide | | | |
| N-(4-bromo-3-(4H-1,2,4-triazol-3-yl)thiophen-2-yl)- | (++) | (++) | (++) |
| 2-(7-(trifluoromethyl)quinolin-5-yl)acetamide | | | |
| N-(4-bromo-3-(4H-1,2,4-triazol-3-yl)thiophen-2-yl)- | (++) | (++) | (++) |
| 2-(5-(trifluoromethyl)quinolin-7-yl)acetamide | | | |
| N-(4-bromo-3-(1H-1,2,4-triazol-5-yl)thiophen-2-yl)- | (+++) | (+++) | (+++) |
| 2-(2-oxo-1,5-naphthyridin-1 (2H)-yl)acetamide | | | |
| N-(4-bromo-3-(1H-1,2,4-triazol-5-yl)thiophen-2-yl)- | (+++) | (+++) | (+++) |
| 2-(2-oxo-3,4-dihydro-1,6-naphthyridin-1 (2H)- | | | |
| yl)acetamide | | | |
| N-(4-bromo-3-(1H-1,2,4-triazol-5-yl)thiophen-2-yl)- | (+++) | (+++) | (+++) |
| 2-(2-oxo-3,4-dihydro-1,5-naphthyridin-1 (2H)- | | | |
| yl)acetamide | | | |
| N-(4-bromo-3-(1H-1,2,4-triazol-5-yl)thiophen-2-yl)- | (+++) | (+++) | (+++) |
| 2-(6-chloro-2-oxoquinolin-1 (2H)-yl)acetamide | | | |
| N-(4-bromo-3-(1H-1,2,4-triazol-5-yl)thiophen-2-yl)- | (+++) | (+++) | (+++) |
| 2-(5-fluoro-2-oxoquinolin-1(2H)-yl)acetamide | | | |
| N-(4-bromo-3-(1H-1,2,4-triazol-3-yl)thiophen-2-yl)- | (++) | (+++) | (++) |
| 2-(3-fluoroquinolin-8-yl)acetamide | | | |
| N-(4-bromo-3-(1H-1,2,4-triazol-5-yl)thiophen-2-yl)- | (+++) | (+++) | (+++) |
| 2-(2-oxo-6-(trifluoromethoxy)quinolin-1(2H)- | | | |
| yl)acetamide | | | |
| N-(4-bromo-3-(4H-1,2,4-triazol-3-yl)thiophen-2-yl)- | (+++) | (+++) | (++) |
| 2-(isoquinolin-4-yl)acetamide | | | |
| N-(5-chloro-3-(1H-1,2,4-triazol-5-yl)thiophen-2-yl)- | (++) | (+++) | (++) |
| 2-(2-oxo-6-(trifluoromethyl)quinolin-1 (2H)- | | | |
| N-(4-bromo-3-(1H-1,2,4-triazol-3-yl)thiophen-2-yl)- | (+++) | (+++) | (+++) |
| 2-(3-fluoroquinolin-5-yl)acetamide | | | |
| N-(4-bromo-3-(1H-1,2,4-triazol-5-yl)thiophen-2-yl)- | (+++) | (+++) | (+++) |
| 2-(2-oxo-7-(trifluoromethoxy)quinolin-1 (2H)- | | | |
| yl)acetamide | | | |
| N-(4-bromo-3-(1H-1,2,4-triazol-5-yl)thiophen-2-yl)- | (+++) | (+++) | (+++) |
| 2-(7-cyano-2-oxoquinolin-1 (2H)-yl)acetamide | | | |
| N-(4-bromo-3-(1H-1,2,4-triazol-5-yl)thiophen-2-yl)- | (+++) | (+++) | (++) |
| 2-(isoquinolin-8-yl)acetamide | | | |
| N-(4-bromo-3-(1H-1,2,4-triazol-5-yl)thiophen-2-yl)- | (+++) | (+++) | (+++) |
| 2-(6-cyano-2-oxoquinolin-1 (2H)-yl)acetamide | | | |
| N-(4-bromo-3-(1H-1,2,4-triazol-5-yl)thiophen-2-yl)- | (++) | | (++) |
| 2-(quinolin-8-yl)acetamide | | | |
| N-(4-bromo-3-(1H-1,2,4-triazol-5-yl)thiophen-2-yl) | (+++) | (+++) | (++) |
| 2-(2-oxo-5-(trifluoromethyl)quinolin-1 (2H)- | | | |
| N-(4-bromo-3-(1H-1,2,4-triazol-5-yl)thiophen-2-yl)- | (+++) | (+++) | (+++) |
| 2-(2-oxo-6-(trifluoromethyl)-3,4-dihydroquinolin-1 | | | |
| (2H)-yl)acetamide | | | |
| N-(4-cyano-3-(1-methyl-1H-1,2,4-triazol-3- | (+++) | (+++) | (+++) |
| yl)thiophen-2-yl)-2-(quinolin-5- | | | |
| N-(4-bromo-3-(1H-1,2,4-triazol-3-yl)thiophen-2-yl)- | (++) | (++) | (++) |
| 2-(2-(trifluoromethyl)quinolin-7-yl)acetamide | | | |
| N-(4-bromo-3-(1H-1,2,4-triazol-5-yl)thiophen-2-yl)- | (+++) | (+++) | (++) |
| 2-(7-fluoroquinolin-5-yl)acetamide | | | |
| N-(4-bromo-3-(1H-1,2,4-triazol-3-yl)thiophen 2-yl)- | (+++) | (+++) | (++) |
| 2-(3-(trifluoromethyl)quinolin-5-yl)acetamide | | | |
| N-(4-bromo-3-(1H-1,2,4-triazol-5-yl)thiophen-2-yl)- | (+++) | (+++) | (+++) |
| 2-(6-fluoroquinolin-5-yl)acetamide | | | |
| N-(4-bromo-3-(1H-1,2,4-triazol-5-yl)thiophen-2-yl)- | (++) | (+++) | (++) |
| 2-(6-fluoroquinolin-7-yl)acetamide | | | |
| N-(4-bromo-3-(1H-1,2,4-triazol-5-yl)thiophen-2-yl)- | (+++) | (+++) | (+++) |
| 2-(6-ethynyl-2-oxoquinolin-1 (2H)-yl)acetamide | | | |
| N-(4-bromo-3-(1H-1,2,4-triazol-5-yl)thiophen-2-yl)-2- | (+++) | (+++) | (+++) |
| (2-oxo-7-(trifluoromethyl)-1,6-naphthyridin-1 | | | |
| (2H)-yl)acetamide | | | |
| N-(4-bromo-3-(1H-1,2,4-triazol-5-yl)thiophen-2-y)- | (+++) | (+++) | (+++) |
| 2-(5-oxopyrazolo[1,5-a]pyrimidin-4(5H)-yl)acetam | | | |
| [symbol] | | | |
| N-(4-bromo-3-(1H-1,2,4-triazol-3-yl)thiophen 2-yl)-2- | (++) | (++) | (++) |
| (3-(trifluoromethyl)quinolin-8-yl)acetamide | | | |
| N-(4-bromo-3-(1H-1,2,4-triazol-5-yl)thiophen-2-yl)-2- | (++) | (+++) | (++) |
| (6-methylimidazo[2,1-b]thiazol-3-yl)acetamide | | | |
| 2-(2-oxo-6-(trifluoromethyl)quinolin-1 (2H)-yl)-N-(2- | (+++) | (+++) | (++) |
| (thiazol-4-yl)thiophen-3-yl)acetamide | | | |
| N-(4-cyano-3-(pyrazin-2-yl)thiophen-2-yl)-2-(quinolin- | (+++) | (+++) | (++) |
| 5-yl)acetamide | | | |
| 2-(2-oxo-1,6-naphthyridin-1 (2H)-yl)-N-(2-(thiazol-4- | (++) | (+++) | (++) |
| yl)thiophen-3-yl)acetamide | | | |
| N-(4-bromo-3-(4H-1,2,4-triazol-3-yl)thiophen-2-yl)- | (++) | (+++) | (++) |
| 2-(3,3-difluoro-2-oxoindolin-1-yl)acetamide | | | |
| 2-(benzo[d]thiazol-7-yl)-N-(4-bromo-3-(1H- | (+++) | (+++) | (++) |
| 1,2,4-triazol-5-yl)thiophen-2-yl)acetamide | | | |
| N-(4-chloro-3-(1H-1,2,4-triazol-5-yl)thiophen-2-yl)- | (+++) | (+++) | (+++) |
| 2-(2-oxo-6-(trifluoromethyl)quinolin-1 (2H)- | | | |
| N-(4-cyano-3-(oxazol-2-yl)thiophen-2-yl)-2-(2-oxo- | (+++) | (+++) | (+++) |
| 6-(trifluoromethyl)quinolin-1 (2H)- | | | |
| N-(4-bromo-3-(1H-1,2,4-triazol-5-yl)thiophen-2-yl)- | (+++) | (+++) | (+++) |
| 2-(2-oxo-6-(trifluoromethyl)-1,5-naphthyridin-1 | | | |
| (2H)-yl)acetamide | | | |
| N-(3-(1,2,4-oxadiazol-3-yl)thiophen-2-yl)-2-(6, | (++) | (+++) | (++) |
| 7-difluoro-2-oxoquinolin-1 (2H)- | | | |
| N-(4-cyano-3-(thiazol-2-yl)thiophen-2-yl)-2-(2-oxo- | (+++) | (+++) | (+++) |
| 6-(trifluoromethyl)quinolin-1 (2H)- | | | |
| N-(4-bromo-3-(1H-1,2,4-triazol-5-yl)thiophen-2-yl)- | (+++) | (+++) | (+++) |
| 2-(5-oxo-2-(trifluoromethyl)pyrazolo[1,5- | | | |
| a]pyrimidin-4(5H)-yl)acetamide | | | |
| N-(4-cyano-3-(thiazol-4-yl)thiophen-2-yl)-2-(2-oxo- | (+++) | (+++) | (+++) |
| 6-(trifluoromethyl)quinolin-1(2H)- | | | |
| N-(4-bromo-3-(1H-1,2,4-triazol-5-yl)thiophen-2-yl)- | (++) | (+++) | (++) |
| 2-(imidazo[1,2-a]pyridin-5-yl)acetamide | | | |
| N-(4-bromo-3-(1H-1,2,4-triazol-5-yl)thiophen-2-yl)- | (+++) | (+++) | (+++) |
| 2-(7-fluoro-2-oxo-6-(trifluoromethyl)quinolin-1 | | | |
| (2H)-yl)acetamide | | | |
| N-(4-bromo-3-(1-methyl-1H-1,2,4-triazol-3- | (+++) | (+++) | (+++) |
| yl)thiophen-2-yl)-2-(2-oxo-3,4-dihydro- | | | |
| 1,5-naphthyridin-1 (2H)- | | | |
| N-(4-cyano-3-(thiazol-5-yl)thiophen-2-yl)-2-(2-oxo- | (++) | (++) | (++) |
| 6-(trifluoromethyl)quinolin-1 (2H)- | | | |
| N-(4-cyano-3-(1H-1,2,3-triazol-1-yl)thiophen-2-yl)- | (++) | (++) | (++) |
| 2-(2-oxo-6-(trifluoromethyl)quinolin-1 (2H)- | | | |
| N-(3-(1H-benzo[d][1,2,3]triazol-1-yl)-4- | (++) | (++) | (++) |
| cyanothiophen-2-yl)-2-(2-oxo-6-(trifluoromethyl)quinolin- | | | |
| 1 (2H)-yl)acetamide | | | |
| N-(4-bromo-3-(1H-1,2,4-triazol-5-yl)thiophen-2-yl)- | (+++) | (+++) | (+++) |
| 2-(6-fluoroisoquinolin-5-yl)acetamide; and N-(4- | | | |
| bromo-3- | | | |
| (1H-1,2,4-triazol-5-yl)thiophen-2-yl)-2-( | | | |
| N-(4-cyano-3-(2H-1,2,3-triazol-2-yl)thiophen-2-yl)-2- | (+++) | (+++) | (+++) |
| (2-oxo-6-(trifluoromethyl)quinolin-1 (2H)- | | | |
| N-(4-chloro-3-(1H-1,2,4-triazol-3-yl)thiophen-2-yl)- | (+++) | (+++) | (+++) |
| 2-(2-oxo-3,4-dihydro-1,5-naphthyridin-1 (2H)- | | | |
| yl)acetamide | | | |
| N-(4-cyano-3-(thiazol-2-yl)thiophen-2-yl)-2-(2-oxo- | (+++) | (+++) | (+++) |
| 3,4-dihydro-1,5-naphthyridin-1 (2H)- | | | |
| N-(4-chloro-3-(1-methyl-1H-1,2,4-triazol-3- | (+++) | (+++) | (+++) |
| yl)thiophen-2-yl)-2-(2-oxo-3,4-dihydro-1,5- | | | |
| naphthyridin-1 (2H)-yl)acetamide | | | |
| N-(4-chloro-3-(1H-1,2,4-triazol-5-yl)thiophen-2-yl)- | (+++) | (+++) | (+++) |
| 2-(5-oxopyrazolo[1,5-a]pyrimidin-4(5H)- | | | |
| N-(4-bromo-3-(oxazol-2-yl)thiophen-2-yl)-2-(2- | (+++) | (+++) | (+++) |
| oxo-3,4-dihydro-1,5-naphthyridin-1 (2H)- | | | |
| N-(4-bromo-3-(thiazol-2-yl)thiophen-2-yl)-2-(2- | (+++) | (+++) | (++) |
| oxo-3,4-dihydro-1,5-naphthyridin-1 (2H)- | | | |
| N-(4-chloro-3-(thiazol-2-yl)thiophen-2-yl)-2-(2- | (+++) | (+++) | (+++) |
| oxo-3,4-dihydro-1,5-naphthyridin-1 (2H)- | | | |
| N-(3-(benzo[d]thiazol-2-yl)-4-cyanothiophen-2-yl)-2- | (++) | (++) | (++) |
| (2-oxo-3,4-dihydro-1,5-naphthyridin-1 (2H)- | | | |
| N-(4-chloro-3-(3-methyl-1H-1,2,4-triazol-5- | (+++) | (+++) | (+++) |
| yl)thiophen-2-yl)-2-(2-oxo-3,4-dihydro-1,5- | | | |
| naphthyridin-1 (2H)-yl)acetamide | | | |
| N-(4-chloro-3-(1-(3-(dimethylamino)propyl)-1H-1,2,4- | (+++) | (+++) | (+++) |
| triazol-3-yl)thiophen-2-yl)-2-(2-oxo-6- | | | |
| (trifluoromethyl)quinolin-1 (2H)- | | | |
| N-(4-chloro-3-(1-(3-(4-methylpiperazin-1-yl)propyl)- | (+++) | (+++) | (++) |
| 1H-1,2,4-triazol-3-yl)thiophen-2-yl)-2-(2-oxo- | | | |
| 6-(trifluoromethyl)quinolin-1 (2H)- | | | |
| N-(4-chloro-3-(3-ethyl-1H-1,2,4-triazol-5- | (+++) | (+++) | (++) |
| yl)thiophen-2-yl)-2-(2-oxo-3,4-dihydro-1,5- | | | |
| naphthyridin-1 (2H)-yl)acetamide | | | |
| N-(4-chloro-3-(1-(2-(dimethylamino)ethyl)-1H-1,2,4- | (+++) | (+++) | (+++) |
| triazol-3-yl)thiophen-2-yl)-2-(2-oxo-6- | | | |
| (trifluoromethyl)quinolin-1 (2H)- | | | |
| N-(4-chloro-3-(1H-1,2,4-triazol-3-yl)thiophen-2-yl)- | (++) | (++) | (++) |
| N-(2-(dimethylamino)ethyl)-2-(2-oxo-6- | | | |
| (trifluoromethyl)quinolin-1 (2H)-yl)acetamide | | | |
| N-(4-cyano-3-(1H-1,2,4-triazol-3-yl)thiophen-2-yl)- | (+++) | (+++) | (+++) |
| 2-(2-oxo-3,4-dihydro-1,5-naphthyridin-1 (2H)- | | | |
| yl)acetamide | | | |
| N-(4-chloro-3-(1-methyl-1H-1,2,4-triazol-3- | (+++) | (+++) | (+++) |
| yl)thiophen-2-yl)-2-(5-oxopyrazolo[1,5-a]pyrimidin- | | | |
| 4(5H)-yl)acetamide | | | |
| N-(4-chloro-3-(1-(3-morpholinopropyl)-1H-1,2,4- | (+++) | (+++) | (+++) |
| triazol-3-yl)thiophen-2-yl)-2-(2-oxo-6- | | | |
| (trifluoromethyl)quinolin-1 (2H)- | | | |
| N-(4-chloro-3-(1H-1,2,4-triazol-3-yl)thiophen-2-yl)- | (++) | (++) | (++) |
| 2-(2-oxo-6-(trifluoromethyl)quinolin-1 (2H)-yl)-N- | | | |
| (3-(pyrrolidin-1-yl)propyl)acetamide | | | |
| N-(4-chloro-3-(1-(3-(pyrrolidin-1-yl)propyl)-1H- | (+++) | (+++) | (+++) |
| 1,2,4-triazol-3-yl)thiophen-2-yl)-2-(2-oxo-6- | | | |
| (trifluoromethyl)quinolin-1 (2H)-yl)acetamide | | | |
| N-(4-chloro-3-(1-methyl-1H-1,2,4-triazol-3- | (+++) | (+++) | (+++) |
| yl)thiophen-2-yl)-2-(2-oxo-6-(trifluoromethyl)-1,5- | | | |
| naphthyridin-1(2H)-yl)acetamide | | | |
| N-(4-chloro-3-(1H-1,2,4-triazol-5-yl)thiophen-2-yl)- | (++) | (+++) | (++) |
| 2-(8-(trifluoromethyl)quinolin-5-yl)acetamide | | | |
| N-(4-bromo-3-(2H-1,2,3-triazol-2-yl)thiophen-2-yl)- | (+++) | (++) | (+++) |
| 2-(2-oxo-6-(trifluoromethyl)quinolin-1 (2H)- | | | |
| N-(4-chloro-3-(1H-1,2,4-triazol-5-yl)thiophen-2-yl)- | (+++) | (+++) | (+++) |
| 2-(2-oxo-6-(trifluoromethyl)-1,5-naphthyridin-1 | | | |
| (2H)-yl)acetamide | | | |
| N-(4-chloro-3-(3-isopropyl-1H-1,2,4-triazol-5- | (++) | (+++) | (++) |
| yl)thiophen-2-yl)-2-(2-oxo-3,4-dihydro- | | | |
| 1,5-naphthyridin-1 (2H)-yl)acetamide | | | |
| 2-(6-bromo-2-oxoquinolin-1(2H)-yl)-N-(4-bromo- | (+++) | (+++) | (+++) |
| 3-(1H-1,2,4-triazol-3-yl)thiophen-2- | | | |
| N-(4-cyano-3-(1-methyl-1H-1,2,4-triazol-3- | (+++) | (+++) | (+++) |
| yl)thiophen-2-yl)-2-(2-oxo-3,4-dihydro-1,5- | | | |
| naphthyridin-1 (2H)-yl)acetamide | | | |
| 2-(6-bromo-2-oxoquinolin-1 (2H)-yl)-N-(4-chloro- | (+++) | (+++) | (+++) |
| 3-(1H-1,2,4-triazol-3-yl)thiophen-2-yl)acetamide | | | |
| N-(4-chloro-3-(1H-1,2,4-triazol-3-yl)thiophen-2-yl)- | (+++) | (+++) | (+++) |
| 2-(6-cyano-2-oxoquinolin-1 (2H)-yl)acetamide | | | |
| N-(4-chloro-3-(1H-1,2,4-triazol-5-yl)thiophen-2-yl)- | (+++) | (+++) | (+++) |
| 2-(5-oxo-2-(trifluoromethyl)pyrazolo[1,5- | | | |
| a]pyrimidin-4(5H)-yl)acetamide | | | |
| N-(4-chloro-3-(1H-1,2,4-triazol-3-yl)thiophen-2-yl)- | (+++) | (+++) | (+++) |
| 2-(2-oxo-7-(trifluoromethyl)-1,6-naphthyridin-1 | | | |
| (2H)-yl)acetamide | | | |
| N-(4-chloro-3-(1-methyl-1H-1,2,4-triazol-3- | (+++) | (+++) | (+++) |
| yl)thiophen-2-yl)-2-(5-oxo-2- | | | |
| (trifluoromethyl)pyrazolo[1,5-a]pyrimidin-4(5H)- | | | |
| N-(4-chloro-3-(1-methyl-1H-1,2,4-triazol-3- | (+++) | (+++) | (+++) |
| yl)thiophen-2-yl)-2-(2-oxo-7-(trifluoromethyl)-1,6- | | | |
| naphthyridin-1(2H)-yl)acetamide | | | |

(+++) IC₅₀ <0.1 μM
(++) IC₅₀ 0.1 μM-10 μM
(+) IC₅₀ >10 μM
(−) Activity below level of detection in assay used (IC₅₀ >50 μM) [symbol]
indicates data missing or illegible when filed

### Pharmaceutical Compositions

The disclosure further provides pharmaceutical compositions including a compound of the present disclosure, e.g., those of Formulae (I) to (XV) (or any embodiment thereof), and at least one pharmaceutically acceptable carrier. The term “pharmaceutically acceptable carrier” means all pharmaceutically acceptable ingredients known to those of skill in the art, which are typically considered non-active ingredients. The term “pharmaceutically acceptable carrier” includes solvents, solid or liquid diluents, vehicles, adjuvants, excipients, glidants, binders, granulating agents, dispersing agents, suspending agents, wetting agents, lubricating agents, disintegrants, solubilizers, stabilizers, emulsifiers, fillers, preservatives (e.g., anti-oxidants), flavoring agents, sweetening agents, thickening agents, buffering agents, coloring agents and the like, as well as any mixtures thereof. Exemplary carriers (i.e., excipients) are described in, e.g., Handbook of Pharmaceutical Manufacturing Formulations, Volumes 1-6, Niazi, Sarfaraz K., Taylor & Francis Group 2005, which is incorporated herein by reference in its entirety. A pharmaceutical composition of the present disclosure may include one or more compounds of the present disclosure in association with one or more pharmaceutically acceptable carrier and optionally other active ingredients.

The compounds of the present disclosure may be administered orally, topically, parenterally, by inhalation or spray or rectally in dosage unit formulations containing at least one pharmaceutically acceptable carrier. The term “parenteral” as used herein includes percutaneous, subcutaneous, intravascular (e.g., intravenous), intramuscular, or intrathecal injection or infusion techniques and the like. The pharmaceutical compositions containing compounds of the present disclosure may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents chosen from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil. Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of the present disclosure may be administered parenterally in a sterile medium. The compound, depending on the vehicle and concentration used, can either be suspended or dissolved in the vehicle. In one embodiment, adjuvants such as local anesthetics, preservatives and buffering agents can be dissolved in the vehicle.

For disorders of the eye or other external tissues, e.g., mouth and skin, the formulations are applied, for example, as a topical gel, spray, ointment or cream, or as a scleral suppository, containing the active ingredients in a total amount of, for example, 0.075 to 30% w/w, 0.2 to 20% w/w or such as 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound, which enhances absorption or penetration of the active ingredient through the skin or other affected areas. Examples of such dermal penetration enhancers include dimethylsulfoxide and related analogs. The compounds of this present disclosure can also be administered by a transdermal device. In one embodiment, topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this present disclosure may be constituted from known ingredients in a known manner. While the phase may comprise merely an emulsifier, it may comprise a mixture of at least one emulsifier with a fat or oil or with both a fat and an oil. In one embodiment, a hydrophilic emulsifier is included together with a lipophilic emulsifier, which acts as a stabilizer. The phase may, for example, include both an oil and a fat. Together, the emulsifier(s) with or without stabilizer(s) make-up the so-called emulsifying wax, and the wax together with the oil and fat make up the so-called emulsifying ointment base, which forms the oily, dispersed phase of the cream formulations. Emulsifiers and emulsion stabilizers suitable for use in the formulation of the present disclosure include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream may, for example, be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The anti-inflammatory active ingredients may, for example, be present in such formulations in a concentration of 0.5 to 20%, such as 0.5 to 10%, for example about 1.5% w/w. For therapeutic purposes, the active compounds of the present disclosure are ordinarily combined with one or more adjuvants appropriate to the indicated route of administration. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.005 mg to about 80 mg per kilogram of body weight per day are useful in the treatment of the diseases and conditions described herein (e.g., about 0.35 mg to about 5.6 g per human patient per day, based on an average adult person weight of 70 kg). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may, for example, be applied as a topical preparation of compounds of this present disclosure on the affected area one to four times a day.

Formulations suitable for inhalation or insufflation include solutions and suspensions in pharmaceutically acceptable aqueous or organic solvents, or mixtures thereof, and powders. The liquid or solid compositions may contain suitable pharmaceutically acceptable excipients as describe above. The compositions may be administered by oral or nasal respiratory route for local or systemic effect. Compositions may be nebulized by use of inert gases or vaporized, and breathed directly from the nebulizing/vaporizing device or the nebulizing device may be attached to a facemask tent or intermittent positive pressure-breathing machine.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water.

In addition to the compounds of this disclosure, pharmaceutically acceptable derivatives or prodrugs of the compounds of this disclosure may also be employed in compositions to treat or prevent the above-identified disorders. In some embodiments, the composition is co-administered with an antifibrotic agent, selected from the group consisting of: pentoxyphiline, tocopherol, vitamin E, pioglitazone, INT 747, peginterferon 2b, infliximab, ribavirin, glycyrrhizin, candesartan, losartan, irbesartan, ambrisentan, FG-3019, warfarin, insulin, colchicines, peginterferon 2a, etanercept, pirfenidone, nintedanib, and IL-10.

The disclosures in this document of all articles and references, including patents, are incorporated herein by reference in their entirety.

The instant disclosure is illustrated further by the following examples, which are not to be construed as limiting the present disclosure in scope or spirit to the specific procedures described in them. Analogous methods and compositions within the scope of the present disclosure will be apparent to those skilled in the art.

### EXAMPLES

### Example 1: IGP001 and IGP002 Plasma Compound Levels Exceed Cellular EC₅₀'s in Rat PK/PD Study

Plasma compound levels for two of the JNK inhibitors, IGP001 (Formula (XXI)) and IGP002 (Formula (XXII)), were obtained using a rat model for pharmaceutical administration of the compounds. Female Wistar rats, 7-9 weeks old, were obtained (SLAC Laboratory Animal Co. Ltd., Shanghai, China), and three groups of animals (n=3 each) received 1) kainic acid and 2) either IGP001 in vehicle, IGP002 in vehicle, or vehicle alone. The animals received the kainic acid 60 minutes after they received the IGP001 in vehicle, IGP002 in vehicle, and or vehicle alone.

For administration, the kainic acid was dissolved in saline, and introduced to the rats using the protocol as generally disclosed in Tse K, Puttachary S, Beamer E, Sills G J, Thippeswamy T (2014) PLoS One 9:e96622. Animals in each group were surgically implanted with catheters in carotid artery using polyurethane tubing and heparin (50 IU/mL)/glucose (50%) solutions as the lumen lock solution. Anaesthetic barbital was used during the surgery. The animals were allowed to recover seven days after surgery.

The animals were euthanized 4 hours post kainic acid administration using CO₂ inhalation and blood was collected via cardiac puncture. 5 mL blood was transferred into pre-labled plastic microcentrifuge tubes containing K₂-EDTA (0.5M) as anti-coagulant. Plasma levels were determined by a validated LC/MS analysis.

The levels of IGP001 and IGP002 detected in the plasma of the treated rats are shown in FIG. 2. The results of the plasma concentration studies demonstrated that there is sufficient compound in the plasma of treated animals to exceed cellular EC₅₀ in the periphery. Thus, IGP001 and IGP002 are present in the periphery of treated animals at clinically relevant doses.

### Example 2: Both IGP001 and IGP002 Reduce Jun Phosphorylation in PBMC in a Rat PK/PD Study

Blood from the animals treated with kainic acid as per Example 1 above was processed for the characterization of peripheral blood mononuclear cells (PBMC) from the treated animals. Briefly, the final blood samples from the exsanguinated rats were immediately centrifuged at 3,000 g for 10 minutes at 4° C. The plasma in upper layer was removed, and the remaining portion used for the isolation of the PBMC.

The buffy coat of PBMCs at the erythrocyte-plasma interface and the top red cell layer were collected into a 15 mL falcon tube containing 3 ml chilled PBS. The sample was resuspended and transferred to other 15 mL falcon tubes containing 6 ml Ficoll-Paque plus density gradient, and centrifuged at 18° C. for 10 minutes at 800 g. This resulted in three layers: plasma (upper), Ficoll (middle) and RBCs (lower). The middle ‘cloudy’ interface layer between plasma and Ficoll containing the PBMCs was transferred to a 15 mL tube, washed with 10 mL of cold PBS (4° C., 1×PBS), and centrifuged for 5 min at 4° C. at 1000 g. The supernatant was discarded.

The isolated PBMC were subjected to an ELISA assay using a kit for the detection of c-Jun phosphorylation at serine 63. Detection of phospho-Jun was carried out for the PMBCs of each kainic acid administered rat group: those receiving IGP001 in vehicle, those receiving IGP002 in vehicle, or those receiving vehicle alone.

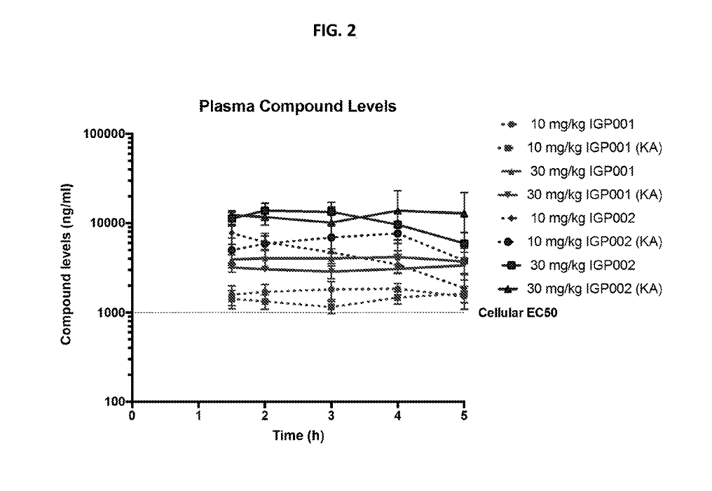

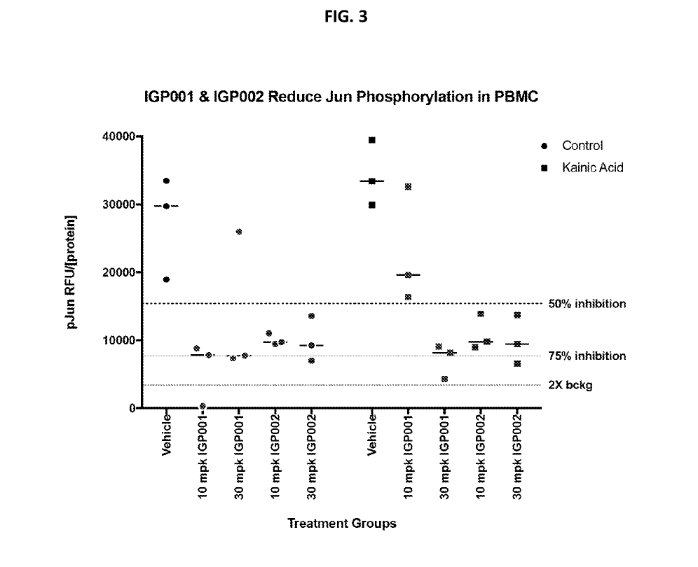

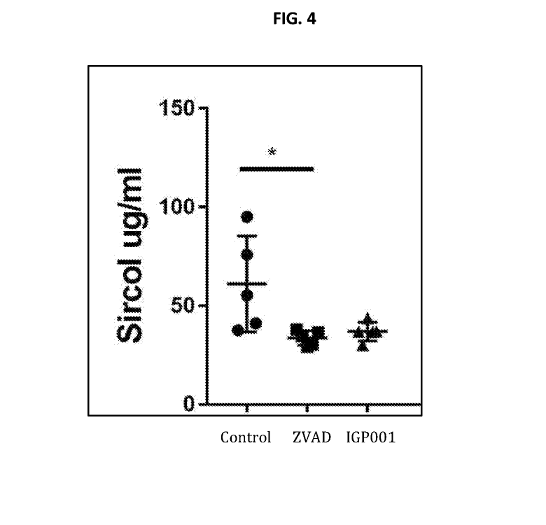

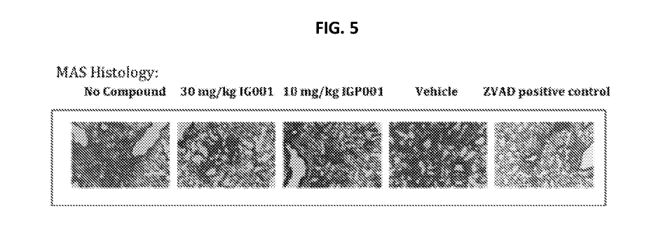

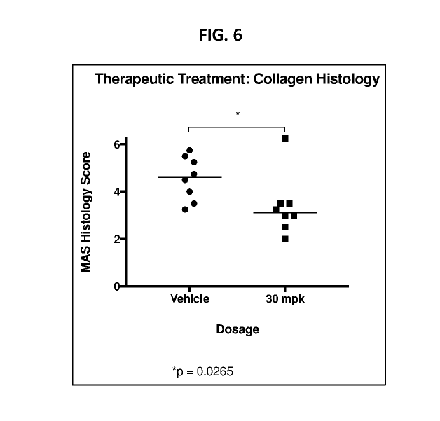

The results, as shown in FIG. 3, demonstrate that a decrease in c-Jun phosphorylation consistent with efficacious target engagement of JNK in the periphery was achieved for both IGP001 and IGP002 in PBMC at 10 and 30 mg/kg. This range confirms the viability of IGP001 and IGP002 to engage with and modulate JNK in the mammalian periphery. Thus JNK inhibition of c-Jun phosphorylation in the periphery is achievable with both IGP001 and IGP002 at clinically relevant doses.

### Example 3: IGP001 and IGP002 Suppress TGF-β1-Induced JNK, but not p38 MAP Kinase or ERK Activation in Human Lung Fibroblasts

IGP001 and IGP002 are shown to affect TGF-β1-induced MAP kinase activities in human lung fibroblasts. Human fetal lung fibroblasts (HFL-1) (ATCC, Rockville, Md.) are cultured in Ham's F12K medium (American Type Culture Collection) supplemented with 50 U/ml penicillin G sodium, 50 μg/ml streptonycin sulfate (GIBCO BRL, Life Technologies, Inc., Rockville, Md.) and 10% fetal bovine serum (FBS; Equitech-Bio. Inc., Ingram, Tex.) and maintained in humidified 5% CO₂ at 37° C. After reaching confluence, the serum content of the medium is reduced to 0.4% FBS for 24 h. The cells are then stimulated with 1 ng/ml TGF-β1 to produce CTGF.

To test the ability of IGP001 and IGP002 to suppress activation of JNK, HFL-1 cells are cultured with 0.1% DMSO (control vehicle), IGP001 or IGP002 at a concentration of 30 mg/kg for 30 minutes, followed by stimulation with TGF-β1 (1 ng/ml) for 30 minutes. Activities of JNK, p38 MAP kinase and ERK are determined by Western blotting whole cell lysates using antibodies specific for phosphorylated, activated forms of JNK, p38 MAP kinase, and ERK. Two identical experiments independently performed yielded similar results.

Phosphorylated JNK protein levels are quantified by densitometry. The optical density of the p46 and p54 phosphorylated protein band are corrected for total p46 and p54 protein, respectively, and results are expressed as ratio (%) of control value (TGF-β1-stimulated HFL-1 cells). Both IGP001 and IGP002 suppress TGF-β1-induced JNK phosphorylation in a dose-dependent fashion. These results demonstrate that IGP001 and IGP002 can inhibit the JNK regulation of TGF-β1-induced CTGF expression.

### Example 4: IGP001 and IGP002 Suppress TGF-β31-Induced SMAD2 Phosphorylation

TGF-β1 factors initiate signaling by assembling receptor complexes that activate SMAD proteins. IGP001 and IGP002 are tested for their ability to suppress TGF-031 induction of SMAD signaling pathway by detecting the dually phosphorylated (Ser 465/Ser 467) form of SMAD2 by Western blotting against specific anti-phosphokinase antibody.

HFL-1 cells are created as described above in Example 3. HFL-1 cells are cultured with 0.1% DMSO (control vehicle), IGP001 or IGP002 for 30 min before stimulation with TGF-β1 (1 ng/ml) for 4 hours. Total RNA is extracted and hybridized with human CTGF probes, and messenger RNA levels are quantified by densitometry and optical density of CTGF mRNA band was corrected for G3PDH, and results are expressed as ratio (%) of control value (TGF-β1-stimulated HFL-1 cells).

Phosphorylation of SMAD2 following treatment with IGP001 or IGP002 are also determined by Western blotting whole cell lysates using an antibody specific for phosphorylated forms of SMAD2.

In HFL-1 cells treated with IGP001 and IGP002, amounts of phosphorylated SMAD2 significantly increase, reaching a maximum at 60 minutes after adding TGF-β1. These JNK inhibitors suppressed CTGF mRNA expression by HFL-1 cells stimulated with TGF-β1 in a dose-dependent fashion.

### Example 5: Prevention of Experimentally Induced Lung Fibrosis Using an Orally Administered JNK Inhibitor

The JNK inhibitors were also show efficacy in the treatment of lung fibrosis in a bleomycin injury model of idiopathic lung fibrosis.

An experiment was designed to determine whether an orally administered JNK inhibitor can prevent experimentally induced lung fibrosis. Age and gender-matched wild type mice on the C57BL/6 background were utilized for this study, which is outlined in Table 1:

TABLE 1
Experimental Plan

| Experimental | |
|---|---|
| Agent | IT bleo |
| IGP001 10 mg/kg | n = 10 |
| b.i.d. | |
| IGP001 30 mg/kg | n = 10 |
| b.i.d. | |
| No Compound | n = 10 |
| control | |
| Negative Control | n = 10 |
| Compound | |
| ZVAD apoptosis | n = 10 |
| inhibitor, i.p. | |

Mice are housed in a sterile barrier facility at the Yale University School of Medicine and all proposed procedures are approved on existing mouse protocols. Mice were given a single inhaled dose of 1.25 Units/kg pharmacologic grade bleomycin (Teva Pharmaceuticals, Israel) in a volume of 50 μl. Mice received twice daily administered IGP001, via oral gavage, on a b.i.d. schedule starting prior to bleomycin treatment in this preventative study. Mice were sacrificed at 14 days following the administration of bleomycin and assessed as appropriate for the outcomes shown in Table 2:

TABLE 2
Outcomes of interest

```
Sircol Assay
    Total soluble collagen content
Lung Histology
    Masson's Trichrome Stains -
    deposited collagen
```

Briefly, lung inflammation was assessed by Bronchoalveolar lavage cell counts. Total soluble lung collagen will be quantified using Sircol Assay (Biocolour, Ireland). Formalin fixed, paraffin embedded mouse lung sections were stained with hematoxylin and eosin to assess gross morphology, morphometry, and Ashcroft scores and with Masson's trichrome stains to visualize collagen deposition.

As shown in FIG. 1, 10 mg/kg IGP001 worked as well as ZVAD, an apoptosis inhibitor, in reducing bleomycin induced soluble collagen levels in female lungs. Both 10 mg/kg and 30 mg/kg IGP001 reduced deposited collagen as demonstrated in FIG. 2, which show tissue samples visualized using Masson's trichrome stain compared to No Compound treated and Negative control/vehicle treated lungs. Data was reported as the mean+/−SEM and assessed for significance by Student's t-test or analysis of variance as appropriate. Data that are not normally distributed will be assessed for significance using the Mann-Whitney U test. Significance will be determined by p-values ≤0.05.

This experiment demonstrated that IGP001 administration can prevent lung fibrosis in a belomycin model of IPF.

### Example 6: Treatment of Experimentally Induced Lung Fibrosis Using an Orally Administered JNK Inhibitors

A subsequent experiment was performed to determine whether an orally administered JNK inhibitor can reduce experimentally induced lung fibrosis in a therapeutic model, i.e. in mice with previously induced IPF. Age and gender-matched wild type mice on the C57BL/6 background were utilized for this study, as outlined in Table 3:

TABLE 3
Experimental Plan

| Experimental | |
|---|---|
| Agent | IT bleo |
| IGP001 30 mg/kg | n = 8 |
| b.i.d. | |
| Vehicle control | n = 8 |

Mice were housed in a sterile barrier facility and given a single inhaled dose of 1.25 Units/kg pharmacologic grade bleomycin (Teva Pharmaceuticals, Israel) in a volume of 50 μl. Mice received twice daily administered IGP001, via oral gavage, on a b.i.d. schedule starting 7 days after bleomycin treatment in this therapeutic study. Mice were sacrificed at 14 days following the administration of bleomycin and assessed as appropriate for deposited collagen in the lungs using Masson's Trichrome stain.

As shown in FIG. 3, 30 mg/kg IGP001 significantly reduced deposited collagen in the lungs of both male and female mice in this study compared to vehicle controls. Data was reported as the mean+/−SEM and assessed for significance by Student's t-test or analysis of variance as appropriate. Data that were not normally distributed were assessed for significance using the Mann-Whitney U test. Significance was determined by p-values ≤0.05.

### Example 7: Treatment of Experimentally Induced Kidney Fibrosis Using JNK Inhibitor Treatment

To study the effects of JNK inhibitors on development of long-term fibrosis after acute kidney injury, a mouse model of unilateral kidney IRI is established (Furuichi et al., 2006; Feitoza et al., 2008; Ko et al., 2010). Clamping of one, rather than both, renal pedicle(s) permits prolongs survival of experimental mice and thus allows evaluation of late fibrosis and renal function (Furuichi et al., 2006; Feitoza et al., 2008; Ko et al., 2010). The left renal pedicle is cross-clamped for 45 min (Furuichi et al., 2006; Feitoza et al., 2008; Ko et al., 2010). Upon release of the clamp, the kidney is observed to ensure that reperfusion occurs. Sham surgery is similarly performed, without cross-clamping the renal vessels. FUA prolonged period of ischemia (e.g., 45 min) at the outset is needed to produce sufficient injury to cause late interstitial fibrosis (Furuichi et al., 2006; Feitoza et al., 2008; Ko et al., 2010). In this model, early fibrotic changes are visible by 14 days and more robust fibrosis is evident at 28 days after induction of acute ischemia (Furuichi et al., 2006; Feitoza et al., 2008; Ko et al., 2010).

Mice are subjected to renal artery clamping and release, or to sham surgery, of only the left kidney (Furuichi et al., 2006; Feitoza et al., 2008; Ko et al., 2010). A significant increase in renal fibrosis is seen in the clamped kidney as early as 14 days.

IGP001, IGP002, (10 mg/kg) or control vehicle is p.o. dosed 1 hour prior to inducing IRI and intraperitoneally (ip) every 3 days thereafter. To study the function of the injured kidney, the uninjured kidney is resected on Day 13. The injured kidney and blood are collected 1 day later, that is, 14 days after IRI (Furuichi et al., 2006; Feitoza et al., 2008; Ko et al., 2010).

Mice are subjected to nephrectomy of the uninjured kidney 1 day prior to measurement of renal function, so that the plasma creatinine and urea reflect the function of the injured kidney, and not that of the uninjured kidney (Furuichi et al., 2006; Ko et al., 2010). Even at this early time, mice that received vehicle control have lower kidney weights than their sham-operated counterparts, reflecting loss of normal kidney tissue and development of more fibrosis. Plasma creatinine and urea concentration are measured using standard autoanalyzer bethods.

To assess fibrosis, the injured kidney is sectioned and stained with picrosirius red (PSR) to identify fibrillar collagen with red staining. Mice that received the JNK inhibitors display preserved kidney weight, less renal fibrosis and better renal function than mice treated with vehicle control.

### Example 8: Treatment of Experimentally Induced Cardiac Fibrosis Using JNK Inhibitors

To study the effects of JNK inhibitors on prevention and/or progression of cardiac infarct size following myocardial infarction, a rat model of cardiac ischemia and remodeling was used. Briefly, adult male Sprague-Dawley rats (Charles River labs) are subjected to 45 minutes normothermic ischemia (left coronary artery occlusion) followed by 48 hours reperfusion. In rodents, myocardial infarct size stabilizes by 48 hour post-ischemia following which events relating to left ventricular (LV) dilatation and remodeling begin. At the onset of reperfusion, animals receive vehicle, IGP001 or IGP002 (10 mg/kg/day) p.o. Infarct size as a percentage of region-at-risk (RAR) is determined using standard Evans Blue and tetrazolium chloride staining of the hearts.

Treatment with IGP001 and IGP002, but not with vehicle, results in a significant reduction in postischemic myocardial infarct size, an indicator of reduced fibrotic activity in the post-infarct heart.

Rats are next tested for ischemic ventricular remodeling and cardiac function following treatment with IGP001 and IGP002. Rats subjected to 45 minutes normothermic myocardial ischemia receive vehicle, IGP001 or IGP002 p.o. (10 mg/kg/day, p.o.; twice/day for remainder of study) at onset of reperfusion. Following a 48 hour treatment period, animals are subjected to echocardiography (Philips Envisor C Ultrasound) at week 1 reperfusion and sacrificed at week 8 reperfusion. Myocardial ischemia-reperfusion is associated with the hall mark indices of LV remodeling: increased end-diastolic volume or LV dilatation; increased end-systolic volume (i.e. reduced cardiac output); reduced fractional shortening and ejection fraction. Treatment of the rats with either IGP001 or IGP002 for 48 hours following myocardial ischemia reduces adverse LV remodeling and improved cardiac function at week 1.

To better mimic the clinical scenario of delayed start of treatment, rats are administered with vehicle, IGP001 or IGP002 (10 mg/kg/day) p.o. 3 hours after the onset of reperfusion following a 45 minute myocardial ischemia. Animals are sacrificed at 48 hours reperfusion. Administration of IGP001 or IGP002, but not vehicle, is associated with a statistically significant decrease in the size of infarction as compared to the rats receiving vehicle alone.

### Example 9: Treatment of Experimentally Induced NASH Using JNK Inhibitors

The ability of IGP001 and IGP002 are tested in the transgenic SREBP-1c model of NASH. SREBP-1c is a lipogenic transcription factor, and overexpression creates a model of congenital lipodystrophy in which severe insulin resistance and diabetes develop secondary to impaired adipose differentiation (Shimomura I et al., Genes Dev 1998; 12: 3182-3194). In these mice, decreased fat tissue with lipid accumulation in the liver is observed, and marked hepatic steatosis occurs by 8 days of age. When fed a standard diet, steatosis, lobular inflammation, and perivenular and pericellular fibrosis develop at 20 weeks (Nakayama H, et al., Metabolism 2007; 56: 470-475; Takahashi Y, World J Gastroenterol. 2012 May 21; 18(19): 2300-2308).

The antifibrotic effects of IGP001 and IGP002 are also evaluated in a thioacetamide-induced rat model of liver fibrosis. Study assessments include body and liver/kidney weight, liver function test, liver/kidney morphology and collagen deposition, fibrogenic gene and protein expression, and pharmacokinetic analyses.

Animals are treated with vehicle, IGP001 or IGP002 (10 mg/kg/day) p.o. Both IGP001 and IGP002 significantly reduce monocyte/macrophage recruitment in vivo. At these doses, IGP001 and IGP002 show antifibrotic effects, with significant reductions in collagen deposition, and collagen type 1 protein and mRNA expression across the two animal models of fibrosis. In the thioacetamide-induced NASH model, IGP001 and IGP002 significantly reduce the non-alcoholic fatty liver disease activity score.

While this invention is satisfied by aspects in many different forms, as described in detail in connection with preferred aspects of the invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present disclosure, as their purpose is to enable the appropriate authorities, as well as the general public, to quickly determine the general nature of the invention. All references cited herein are incorporated by their entirety for all purposes. In the claims that follow, unless the term “means” is used, none of the features or elements recited therein should be construed as means-plus-function limitations pursuant to 35 U.S.C. § 112, 16.

What is claimed is:

1. A method of treating or preventing a JNK-mediated fibrotic disease in a mammal, comprising administering to the subject a therapeutically effective amount of a compound having a structure according to Formula (I): 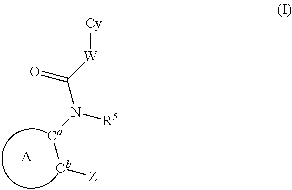
or a salt or solvate thereof, wherein ring A is 5-membered heteroaryl comprising a sulfur atom, wherein the heteroaryl is optionally substituted with 1 or 2 substituents independently chosen from alkyl, alkenyl, alkynyl, haloalkyl, heteroalkyl, C₃-C₁₀-cycloalkyl, 3- to 8-membered heterocycloalkyl, aryl, 5- or 6-membered heteroaryl, CN, halogen, OR¹², SR¹², NR¹²R¹³, C(O)R¹⁴, C(O)NR¹²R¹³, OC(O)NR¹²R¹³, C(O)OR¹², NR¹⁵C(O)R¹⁴, NR¹⁵C(O)OR¹², NR¹⁵C(O)NR¹²R¹³, NR¹⁵C(S)NR¹²R¹³, NR¹⁵S(O)₂R¹⁴, S(O)₂NR¹²R¹³, S(O)R¹⁴ and S(O)₂R¹⁴, wherein R¹², R¹³ and R¹⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹² and R¹³, together with the nitrogen atom to which they are bound form a 5- to 7-membered heterocyclic ring; and R¹⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; C^{a} and C^{b} are carbon atoms, which are adjacent to each other and are part of ring A; Z is 5- or 6-membered heteroaryl, with the proviso that (i) when ring A is thiophene, then Z is not a heteroaryl chosen from benzoimidazole, thiazole, and benzothiazole; (ii) when ring A is thiazole, then Z is not benzoimidazole; (iii) when ring A is thiophene, then Z is not substituted oxadiazole; and (iv) when ring A is thiophene, then Z is not pyrimidinone; R⁵ is chosen from H, acyl, substituted or unsubstituted C₁-C₆ alkyl, and C₃-C₆ cycloalkyl; W is chosen from C₁-C₄ alkylene, wherein the alkylene is optionally substituted with 1-4 substituents independently chosen from alkyl, alkenyl, alkynyl, haloalkyl, heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R⁴⁴, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R⁴⁴, and S(O)₂R⁴⁴, wherein R⁴², R⁴³ and R⁴⁵ are members independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁴² and R⁴³, together with the nitrogen atom to which they are bound are optionally joined to form a 5- to 7-membered heterocyclic ring; and R⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; Cy is chosen from cycloalkyl, heterocycloalkyl, aryl, and heteroaryl, wherein the cycloalkyl, heterocycloalkyl, aryl or heteroaryl is optionally substituted with 1-6 substituents independently chosen from substituted or unsubstituted alkyl, substituted or unsubstituted alkenyl, substituted or unsubstituted alkynyl, haloalkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, CN, halogen, OR⁵², SR⁵², ═O, NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl.

2. The method of claim 1, wherein the compound has a structure according to Formula (II): 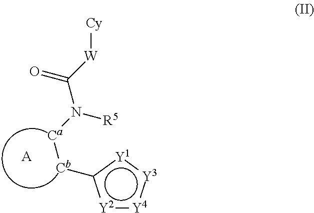
or a salt or solvate
thereof, wherein
Y¹ is chosen from N, O and S; and
Y², Y³ and Y⁴ are independently chosen from S, O, N, NR³ and CR⁴, wherein
each R³ is independently chosen from H, alkyl, alkenyl, alkynyl, haloalkyl, cycloalkyl, 3- to 8-membered heterocycloalkyl, aryl, and 5- or 6-membered heteroaryl; and
each R⁴ is independently chosen from H, alkyl, alkenyl, alkynyl, haloalkyl, cycloalkyl, 3- to 8-membered heterocycloalkyl, aryl, 5- or 6-membered heteroaryl, CN, halogen, OR¹⁷, SR¹⁷ and NR¹⁷R¹⁸,
wherein
R¹⁷ and R¹⁸ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹⁷ and R¹⁸ together with the nitrogen atom to which they are bound are optionally joined to form a 5- to 7-membered heterocyclic ring,
or wherein adjacent R⁴ groups or adjacent R⁴ and R³, together with the atoms to which they are attached, form a 5- to 7-membered ring,
with the proviso that:
(i) at least one of Y¹ and Y² is N;
(ii) when Y³ and Y⁴ are both CR⁴ and Y¹ is N, then Y² is other than S; and
(iii) when Y³ and Y⁴ are both CR⁴ and Y² is N, then Y¹ is other than S.

3. The method of claim 1, wherein ring A is chosen from thiophene, thiazole, and pyrazole, wherein the thiophene, the thiazole, or the pyrazole is optionally substituted with 1 or 2 substituents chosen from C₁-C₄-alkyl, C₁-C₄-alkenyl, C₁-C₄-alkynyl, C₁-C₄-haloalkyl, 2- to 4-membered heteroalkyl, C₃-C₆-cycloalkyl, 3- to 6-membered heterocycloalkyl, CN, and halogen.

4. The method of claim 1, wherein said compound is at least one selected from the group consisting of: N-(4-methyl-3-(1H-1,2,4-triazol-5-yl)thiophen-2-yl)-2-(2-oxo-3,4-dihydroquinolin-1(2H)-yl)acetamide; N-(4-methyl-3-(3-methyl-1H-1,2,4-triazol-5-thiophen-2-yl)-2-(2-oxo-3,4-dihydroquinolin-1(2H)-yl)acetamide; N-(4-chloro-3-(1H-1,2,4-triazol-5-yl)thiophen-2-yl)-2-(2-oxo-3,4-dihydroquinolin-1-(2H)-yl)acetamide; N-(4-chloro-3-(1H-1,2,4-triazol-5-yl)thiophen-2-yl)-2-(isoquinolin-5-yl)acetamide; N-(4-chloro-3-(1H-1,2,4-triazol-5-yl)thiophen-2-yl)-2-(quinolin-5-yl)acetamide; N-(4-methyl-3-(3-methyl-1H-1,2,4-triazol-5-yl)thiophen-2-yl)-2-(quinolin-5-yl)acetamide;
N-(4-bromo-3-(1H-1,2,4-triazol-5-yl)thiophen-2-yl)-2-(isoquinolin-5-yl)acetamide;
N-(4-bromo-3-(1H-1,2,4-triazol-5-yl)thiophen-2-yl)-2-(quinolin-5-yl)acetamide; N-(4-bromo-3-(1H-1,2,4-triazol-5-yl)thiophen-2-yl)-2-(2-oxo-3,4-dihydroquinolin-1(2H)-yl)acetamide; N-(4-cyano-3-(1H-1,2,4-triazol-5-yl)thiophen-2-yl)-2-(isoquinolin-5-yl)acetamide; N-(4-cyano-3-(1H-1,2,4-triazol-5-yl)thiophen-2-yl)-2-(2-oxo-3,4-dihydroquinolin-1(2H)-yl)acetamide; N-(4-cyano-3-(4H-1,2,4-triazol-3-yl)thiophen-2-yl)-2-(quinolin-5-yl)acetamide; N-(4-bromo-3-(1H-1,2,4-triazol-5-yl)thiophen-2-yl)-2-(2-oxo-7-(trifluoromethyl)quinolin-1(2H)-yl)acetamide; N-(4-bromo-3-(1H-1,2,4-triazol-5-yl)thiophen-2-yl)-2-(6-fluoro-2-oxo-3,4-dihydroquinolin-1(2H)-yl)acetamide; N-(4-bromo-3-(1H-1,2,4-triazol-5-yl)thiophen-2-yl)-2-(7-fluoro-2-oxoquinolin-1(2H)-yl)acetamide; N-(4-bromo-3-(1H-1,2,4-triazol-5-yl)thiophen-2-yl)-2-(7-chloro-2-oxo-3,4-dihydroquinolin-1(2H)-yl)acetamide; N-(4-bromo-3-(1H-1,2,4-triazol-5-yl)thiophen-2-yl)-2-(6,7-difluoro-2-oxoquinolin-1(2H)-yl)acetamide; N-(4-bromo-3-(1H-1,2,4-triazol-5-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(6-fluoro-2-oxoquinolin-1(2H)-yl)acetamide; N-(4,4′-bithiazol-5yl)-2(isoquinolin-5yl)acetamide; N-(4-bromo-3-(4H-1,2,4-triazol-3-yl)thiophen-2-yl)-2-(quinolin-4-yl)acetamide; N-(4-bromo-3-(1H-1,2,4-triazol-5-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(8-fluoroisoquinolin-5-yl)acetamide; N-(4-bromo-3-(1H-1,2,4-triazol-5-yl)thiophen-2-yl)-2-(2-oxo-1,6-naphthyridin-1(2H)-yl)acetamide; N-(4-bromo-3-(1H-1,2,4-triazol-5-yl)thiophen-2-yl)-2-(8-fluoroquinolin-5-yl)acetamide; N-(4-bromo-3-(1H-1,2,4-triazol-5-yl)thiophen-2-yl)-2-(2-oxo-1,5-naphthyridin-1(2H)-yl)acetamide; N-(4-bromo-3-(1H-1,2,4-triazol-5-yl)thiophen-2-yl)-2-(2-oxo-3,4-dihydro-1-, 6-naphthyridin-1(2H)-yl)acetamide; N-(4-bromo-3-(1H-1,2,4-triazol-5-yl)thiophen-2-yl)-2-(2-oxo-3,4-dihydro-1-, 5-naphthyridin-1(2H)-yl)acetamide; N-(4-bromo-3-(1H-1,2,4-triazol-5-yl)thiophen-2-yl)-2-(6-chloro-2-oxoquinolin-1(2H)-yl)acetamide; N-(4-bromo-3-(1H-1,2,4-triazol-5-yl)thiophen-2-yl)-2-(5-fluoro-2-oxoquinolin-1(2H)-yl)acetamide; N-(4-bromo-3-(1H-1,2,4-triazol-5-yl)thiophen-2-yl)-2-(2-oxo-6-(trifluoromethoxy)quinolin-1(2H)-yl)acetamide; N-(4-bromo-3-(4H-1,2,4-triazol-3-yl)thiophen-2-yl)-2-(isoquinolin-4-yl)acetamide; N-(4-bromo-3-(1H-1,2,4-triazol-3-yl)thiophen-2-yl)-2-(3-fluoroquinolin-5-yl)acetamide; N-(4-bromo-3-(1H-1,2,4-triazol-5-yl)thiophen-2-yl)-2-(2-oxo-7-(trifluoromethoxy)quinolin-1(2H)-yl)acetamide; N-(4-bromo-3-(1H-1,2,4-triazol-5-yl)thiophen-2-yl)-2-(7-cyano-2-oxoquinolin-1(2H)-yl)acetamide; N-(4-bromo-3-(1H-1,2,4-triazol-5-yl)thiophen-2-yl)-2-(isoquinolin-8-yl)acetamide; N-(4-bromo-3-(1H-1,2,4-triazol-5-yl)thiophen-2-yl)-2-(6-cyano-2-oxoquinolin-1(2H)-yl)acetamide; N-(4-bromo-3-(1H-1,2,4-triazol-5-yl)thiophen-2-yl)-2-(2-oxo-5-(trifluoromethyl)quinolin-1(2H)-yl)acetamide; N-(4-bromo-3-(1H-1,2,4-triazol-5-yl)thiophen-2-yl)-2-(2-oxo-6-(trifluoromethyl)-3,4-dihydroquinolin-1(2H)-yl)acetamide; N-(4-cyano-3-(1-methyl-1H-1,2,4-triazol-3-yl)thiophen-2-yl)-2-(quinolin-5-yl)acetamide; N-(4-bromo-3-(1H-1,2,4-triazol-5-yl)thiophen-2-yl)-2-(7-fluoroquinolin-5-yl)acetamide; N-(4-bromo-3-(1H-1,2,4-triazol-3-yl)thiophen-2-yl)-2-(3-(trifluoromethyl)-quinolin-5-yl)acetamide; N-(4-bromo-3-(1H-1,2,4-triazol-5-yl)thiophen-2-yl)-2-(6-fluoroquinolin-5-yl)acetamide; N-(4-bromo-3-(1H-1,2,4-triazol-5-yl)thiophen-2-yl)-2-(6-ethynyl-2-oxoquinolin-1(2H)-yl)acetamide; N-(4-bromo-3-(1H-1,2,4-triazol-5-yl)thiophen-2-yl)-2-(2-oxo-7-(trifluoromethyl)-1,6-naphthyridin-1(2H)-yl)acetamide; N-(4-bromo-3-(1H-1,2,4-triazol-5-yl)thiophen-2-yl)-2-(5-oxopyrazolo[1,5-a]pyrimidin-4(5H)-yl)acetamide; 2-(2-oxo-6-(trifluoromethyl)quinolin-1(2H)-yl)-N-(2-(thiazol-4-yl)thiophen-3-yl)acetamide; N-(4-cyano-3-(pyrazin-2-yl)thiophen-2-yl)-2-(quinolin-5-yl)acetamide; 2-(benzo[d]thiazol-7-yl)-N-(4-bromo-3-(1H-1,2,4-triazol-5-yl)thiophen-2-yl)acetamide; N-(4-chloro-3-(1H-1,2,4-triazol-5-yl)thiophen-2-yl)-2-(2-oxo-6-(trifluoromethyl)quinolin-1(2H)-yl)acetamide; N-(4-cyano-3-(oxazol-2-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(2-oxo-6-(trifluoromethyl)-1,5-naphthyridin-1(2H)-yl)acetamide; N-(4-cyano-3-(thiazol-2-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(5-oxo-2-(trifluoromethyl)pyrazolo[1,5-a]pyrimidin-4(5H)-yl)acetamide; N-(4-cyano-3-(thiazol-4-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(7-fluoro-2-oxo-6-(trifluoromethyl)quinolin-1(2H)-yl)acetamide; N-(4-bromo-3-(1-methyl-1H-1,2,4-triazol-3-yl)thiophen-2-yl)-2-(2-oxo-3,4-dihydro-1,5-naphthyridin-1(2H)-yl)acetamide; N-(4-bromo-3-(1H-1,2,4-triazol-5-yl)thiophen-2-yl)-2-(6-fluoroisoquinolin-5-yl)acetamide; N-(4-bromo-3-(1H-1,2,4-triazol-5-yl)thiophen-2-yl)-2-(6-fluoroisoquinolin-7-yl)acetamide; N-(4-cyano-3-(2H-1,2,3-triazol-2-yl)thiophen-2-yl)-2-(2-oxo-6-(trifluoromethyl)quinolin-1(2H)-yl)acetamide; N-(4-chloro-3-(1H-1,2,4-triazol-3-yl)thiophen-2-yl)-2-(2-oxo-3,4-dihydro-1,5-naphthyridin-1(2H)-yl)acetamide; N-(4-cyano-3-(thiazol-2-yl)thiophen-2-yl)-2-(2-oxo-3,4-dihydro-1,5-naphthyridin-1(2H)-yl)acetamide; N-(4-chloro-3-(1-methyl-1H-1,2,4-triazol-3-yl)thiophen-2-yl)-2-(2-oxo-3,4-dihydro-1,5-naphthyridin-1(2H)-yl)acetamide; N-(4-chloro-3-(1H-1,2,4-triazol-5-yl)thiophen-2-yl)-2-(5-oxopyrazolo[1,5-a]pyrimidin-4(5H)-yl)acetamide; N-(4-bromo-3-(oxazol-2-yl)thiophen-2-yl)-2-(2-oxo-3,4-dihydro-1,5-naphthyridin-1(2H)-yl)acetamide; N-(4-bromo-3-(thiazol-2-yl)thiophen-2-yl)-2-(2-oxo-3,4-dihydro-1,5-naphthyridin-1(2H)-yl)acetamide; N-(4-chloro-3-(thiazol-2-yl)thiophen-2-yl)-2-(2-oxo-3,4-dihydro-1,5-naphthyridin-1(2H)-yl)acetamide; N-(4-chloro-3-(3-methyl-1H-1,2,4-triazol-5-yl)thiophen-2-yl)-2-(2-oxo-3,4-dihydro-1,5-naphthyridin-1(2H)-yl)acetamide; N-(4-chloro-3-(1-(3-(dimethylamino)propyl)-1H-1,2,4-triazol-3-yl)thiophen-2-yl)-2-(2-oxo-6-(trifluoromethyl)quinolin-1(2H)-yl)acetamide; N-(4-chloro-3-(1-(3-(4-methylpiperazin-1-yl)propyl)-1H-1,2,4-triazol-3-yl-)thiophen-2-yl)-2-(2-oxo-6-(trifluoromethyl)quinolin-1(2H)-yl)acetamide; N-(4-chloro-3-(3-ethyl-1H-1,2,4-triazol-5-yl)thiophen-2-yl)-2-(2-oxo-3,4-dihydro-1,5-naphthyridin-1(2H)-yl)acetamide; N-(4-chloro-3-(1-(2-(dimethylamino)ethyl)-1H-1,2,4-triazol-3-yl)thiophen-2-yl)-2-(2-oxo-6-(trifluoromethyl)quinolin-1(2H)-yl)acetamide; N-(4-cyano-3-(1H-1,2,4-triazol-3-yl)thiophen-2-yl)-2-(2-oxo-3,4-dihydro-1-, 5-naphthyridin-1(2H)-yl)acetamide; N-(4-chloro-3-(1-methyl-1H-1,2,4-triazol-3-yl)thiophen-2-yl)-2-(5-oxopyrazolo[1,5-a]pyrimidin-4(5H)-yl)acetamide; N-(4-chloro-3-(1-(3-morpholinopropyl)-1H-1,2,4-triazol-3-yl)thiophen-2-yl-)-2-(2-oxo-6-(trifluoromethyl)quinolin-1(2H)-yl)acetamide; N-(4-chloro-3-(1-(3-(pyrrolidin-1-yl)propyl)-1H-1,2,4-triazol-3-yl)thiophen-2-yl)-2-(2-oxo-6-(trifluoromethyl)quinolin-1(2H)-yl)acetamide; N-(4-chloro-3-(1-methyl-1H-1,2,4-triazol-3-yl)thiophen-2-yl)-2-(2-oxo-6-(trifluoromethyl)-1,5-naphthyridin-1(2H)-yl)acetamide; N-(4-bromo-3-(2H-1,2,3-triazol-2-yl)thiophen-2-yl)-2-(2-oxo-6-(trifluoromethyl)quinolin-1(2H)-yl)acetamide; N-(4-chloro-3-(1H-1,2,4-triazol-5-yl)thiophen-2-yl)-2-(2-oxo-6-(trifluoromethyl)-1,5-naphthyridin-1(2H)-yl)acetamide; 2-(6-bromo-2-oxoquinolin-1(2H)-yl)-N-(4-bromo-3-(1H-1,2,4-triazol-3-yl)thiophen-2-yl)acetamide; N-(4-cyano-3-(1-methyl-1H-1,2,4-triazol-3-yl)thiophen-2-yl)-2-(2-oxo-3,4-dihydro-1,5-naphthyridin-1(2H)-yl)acetamide; 2-(6-bromo-2-oxoquinolin-1(2H)-yl)-N-(4-chloro-3-(1H-1,2,4-triazol-3-yl)thiophen-2-yl)acetamide; N-(4-chloro-3-(1H-1,2,4-triazol-3-yl)thiophen-2-yl)-2-(6-cyano-2-oxoquinolin-1(2H)-yl)acetamide; N-(4-chloro-3-(1H-1,2,4-triazol-5-yl)thiophen-2-yl)-2-(5-oxo-2-(trifluoromethyl)pyrazolo[1,5-a]pyrimidin-4(5H)-yl)acetamide; N-(4-chloro-3-(1H-1,2,4-triazol-3-yl)thiophen-2-yl)-2-(2-oxo-7-(trifluoromethyl)-1,6-naphthyridin-1(2H)-yl)acetamide; N-(4-chloro-3-(1-methyl-1H-1,2,4-triazol-3-yl)thiophen-2-yl)-2-(5-oxo-2-(trifluoromethyl)pyrazolo[1,5-a]pyrimidin-4(5H)-yl)acetamide; and N-(4-chloro-3-(1-methyl-1H-1,2,4-triazol-3-yl)thiophen-2-yl)-2-(2-oxo-7-(trifluoromethyl)-1,6-naphthyridin-1(2H)-yl)acetamide, or a pharmaceutically acceptable salt thereof.

5. The method of claim 1, wherein the compound according to Formula (I) exhibits an IC₅₀<10 μM.

6. The method of claim 3, wherein the compound according to Formula (I) exhibits an IC₅₀<0.1 μM.

7. A method of treating idiopathic pulmonary fibrosis, organ fibrosis, interstitial lung disease, skin fibrosis, diabetic nephropathy, liver fibrosis, liver cirrhosis, nonalcoholic steatohepatitis (NASH), rheumatoid arthritis, fibrosarcomas, keloids and hypertrophic scars, arteriosclerosis, kidney disease, macular degeneration, retinal and vitreal retinopathy, surgical complications, chemotherapeutic drug-induced fibrosis, radiation-induced fibrosis, accidental injury, burns, local scleroderma, or systemic scleroderma, the method comprising administering to the subject a therapeutically effective amount of a compound having a structure according to Formula (I): 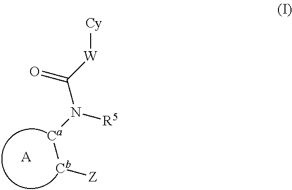
or a salt or solvate thereof, wherein ring A is 5-membered heteroaryl comprising a sulfur atom, wherein the heteroaryl is optionally substituted with 1 or 2 substituents independently chosen from alkyl, alkenyl, alkynyl, haloalkyl, heteroalkyl, C₃-C₁₀-cycloalkyl, 3- to 8-membered heterocycloalkyl, aryl, 5- or 6-membered heteroaryl, CN, halogen, OR¹², SR¹², NR¹²R¹³, C(O)R¹⁴, C(O)NR¹²R¹³, OC(O)NR¹²R¹³, C(O)OR¹², NR¹⁵C(O)R¹⁴, NR¹⁵C(O)OR¹², NR¹⁵C(O)NR¹²R¹³, NR¹⁵C(S)NR¹²R¹³, NR¹⁵S(O)₂R¹⁴, S(O)₂NR¹²R¹³, S(O)R¹⁴ and S(O)₂R¹⁴, wherein R¹², R¹³ and R¹⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹² and R¹³, together with the nitrogen atom to which they are bound form a 5- to 7-membered heterocyclic ring; and R¹⁴ is chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; C^{a} and C^{b} are carbon atoms, which are adjacent to each other and are part of ring A; Z is 5- or 6-membered heteroaryl, with the proviso that (i) when ring A is thiophene, then Z is not a heteroaryl chosen from benzoimidazole, thiazole, and benzothiazole; (ii) when ring A is thiazole, then Z is not benzoimidazole; (iii) when ring A is thiophene, then Z is not substituted oxadiazole; and (iv) when ring A is thiophene, then Z is not pyrimidinone; R⁵ is chosen from H, acyl, substituted or unsubstituted C₁-C₆ alkyl, and C₃-C₆ cycloalkyl; W is chosen from C₁-C₄ alkylene, wherein the alkylene is optionally substituted with 1-4 substituents independently chosen from alkyl, alkenyl, alkynyl, haloalkyl, heteroalkyl, C₃-C₆-cycloalkyl, 3- to 8-membered heterocycloalkyl, aryl, 5- or 6-membered heteroaryl, CN, halogen, OR⁴², SR⁴², NR⁴²R⁴³, C(O)R⁴⁴, C(O)NR⁴²R⁴³, OC(O)NR⁴²R⁴³, C(O)OR⁴², NR⁴⁵C(O)R⁴⁴, NR⁴⁵C(O)OR⁴², NR⁴⁵C(O)NR⁴²R⁴³, NR⁴⁵C(S)NR⁴²R⁴³, NR⁴⁵S(O)₂R⁴⁴, S(O)₂NR⁴²R⁴³, S(O)R⁴, and S(O)₂R⁴⁴, wherein R⁴², R⁴³ and R⁴⁵ are members independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁴² and R⁴³, together with the nitrogen atom to which they are bound are optionally joined to form a 5- to 7-membered heterocyclic ring; and R⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl; Cy is chosen from cycloalkyl, heterocycloalkyl, aryl, and heteroaryl, wherein the cycloalkyl, heterocycloalkyl, aryl or heteroaryl is optionally substituted with 1-6 substituents independently chosen from substituted or unsubstituted alkyl, substituted or unsubstituted alkenyl, substituted or unsubstituted alkynyl, haloalkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, CN, halogen, OR⁵², SR⁵², ═O, NR⁵²R⁵³, C(O)R⁵⁴, C(O)NR⁵²R⁵³, OC(O)NR⁵²R⁵³, C(O)OR⁵², NR⁵⁵C(O)R⁵⁴, NR⁵⁵C(O)OR⁵², NR⁵⁵C(O)NR⁵²R⁵³, NR⁵⁵C(S)NR⁵²R⁵³, NR⁵⁵S(O)₂R⁵⁴, S(O)₂NR⁵²R⁵³, S(O)R⁵⁴ and S(O)₂R⁵⁴, wherein R⁵², R⁵³ and R⁵⁵ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, wherein R⁵² and R⁵³, together with the nitrogen atom to which they are bound are optionally joined to form a 5- to 7-membered heterocyclic ring; and R⁵⁴ is independently chosen from acyl, C₁-C₆-alkyl, 2- to 6-membered heteroalkyl, aryl, 5- or 6-membered heteroaryl, C₃-C₈ cycloalkyl and 3- to 8-membered heterocycloalkyl.

8. The method of claim 7, wherein the compound has a structure according to Formula (II): 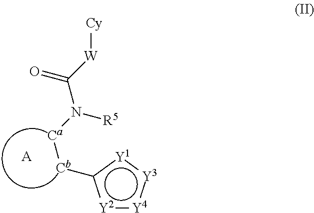
or a salt or solvate
thereof, wherein
Y¹ is chosen from N, O and S; and
Y², Y³ and Y⁴ are independently chosen from S, O, N, NR³ and CR⁴, wherein
each R³ is independently chosen from H, alkyl, alkenyl, alkynyl, haloalkyl, cycloalkyl, 3- to 8-membered heterocycloalkyl, aryl, and 5- or 6-membered heteroaryl; and
each R⁴ is independently chosen from H, alkyl, alkenyl, alkynyl, haloalkyl, cycloalkyl, 3- to 8-membered heterocycloalkyl, aryl, 5- or 6-membered heteroaryl, CN, halogen, OR¹⁷, SR¹⁷ and NR¹⁷R¹⁸,
wherein
R¹⁷ and R¹⁸ are independently chosen from H, acyl, C₁-C₆-alkyl, 2- to 6-membered heteroalkyl, aryl, 5- or 6-membered heteroaryl, C₃-C₈ cycloalkyl and 3- to 8-membered heterocycloalkyl, or R¹⁷ and R¹⁸ together with the nitrogen atom to which they are bound are optionally joined to form a 5- to 7-membered heterocyclic ring,
or wherein adjacent R⁴ groups or adjacent R⁴ and R³, together with the atoms to which they are attached, form a 5- to 7-membered ring,
with the proviso that:
(i) at least one of Y¹ and Y² is N;
(ii) when Y³ and Y⁴ are both CR⁴ and Y¹ is N, then Y² is other than S; and
(iii) when Y³ and Y⁴ are both CR⁴ and Y² is N, then Y¹ is other than S.

9. The method of claim 7, wherein ring A is chosen from thiophene, thiazole, and pyrazole, wherein the thiophene, the thiazole, or the pyrazole is optionally substituted with 1 or 2 substituents chosen from C₁-C₄-alkyl, C₁-C₄-alkenyl, C₁-C₄-alkynyl, C₁-C₄-haloalkyl, 2- to 4-membered heteroalkyl, C₃-C₆-cycloalkyl, 3- to 6-membered heterocycloalkyl, CN, and halogen.

10. The method of claim 7, wherein said compound is at least one selected from the group consisting of: N-(4-methyl-3-(1H-1,2,4-triazol-5-yl)thiophen-2-yl)-2-(2-oxo-3,4-dihydroquinolin-1(2H)-yl)acetamide; N-(4-methyl-3-(3-methyl-1H-1,2,4-triazol-5-thiophen-2-yl)-2-(2-oxo-3,4-dihydroquinolin-1(2H)-yl)acetamide; N-(4-chloro-3-(1H-1,2,4-triazol-5-yl)thiophen-2-yl)-2-(2-oxo-3,4-dihydroquinolin-(2H)-yl)acetamide; N-(4-chloro-3-(1H-1,2,4-triazol-5-yl)thiophen-2-yl)-2-(isoquinolin-5-yl)acetamide; N-(4-chloro-3-(1H-1,2,4-triazol-5-yl)thiophen-2-yl)-2-(quinolin-5-yl)acetamide; N-(4-methyl-3-(3-methyl-1H-1,2,4-triazol-5-yl)thiophen-2-yl)-2-(quinolin-5-yl)acetamide; N-(4-bromo-3-(1H-1,2,4-triazol-5-yl)thiophen-2-yl)-2-(isoquinolin-5-yl)acetamide; N-(4-bromo-3-(1H-1,2,4-triazol-5-yl)thiophen-2-yl)-2-(quinolin-5-yl)acetamide; N-(4-bromo-3-(1H-1,2,4-triazol-5-yl)thiophen-2-yl)-2-(2-oxo-3,4-dihydroquinolin-1(2H)-yl)acetamide; N-(4-cyano-3-(1H-1,2,4-triazol-5-yl)thiophen-2-yl)-2-(isoquinolin-5-yl)acetamide; N-(4-cyano-3-(1H-1,2,4-triazol-5-yl)thiophen-2-yl)-2-(2-oxo-3,4-dihydroquinolin-1(2H)-yl)acetamide; N-(4-cyano-3-(4H-1,2,4-triazol-3-yl)thiophen-2-yl)-2-(quinolin-5-yl)acetamide; N-(4-bromo-3-(1H-1,2,4-triazol-5-yl)thiophen-2-yl)-2-(2-oxo-7-(trifluoromethyl)quinolin-1(2H)-yl)acetamide; N-(4-bromo-3-(1H-1,2,4-triazol-5-yl)thiophen-2-yl)-2-(6-fluoro-2-oxo-3,4-dihydroquinolin-1(2H)-yl)acetamide; N-(4-bromo-3-(1H-1,2,4-triazol-5-yl)thiophen-2-yl)-2-(7-fluoro-2-oxoquinolin-1(2H)-yl)acetamide; N-(4-bromo-3-(1H-1,2,4-triazol-5-yl)thiophen-2-yl)-2-(7-chloro-2-oxo-3,4-dihydroquinolin-1(2H)-yl)acetamide; N-(4-bromo-3-(1H-1,2,4-triazol-5-yl)thiophen-2-yl)-2-(6,7-difluoro-2-oxoquinolin-1(2H)-yl)acetamide; N-(4-bromo-3-(1H-1,2,4-triazol-5-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(6-fluoro-2-oxoquinolin-1(2H)-yl)acetamide; N-(4,4′-bithiazol-5yl)-2(isoquinolin-5yl)acetamide; N-(4-bromo-3-(4H-1,2,4-triazol-3-yl)thiophen-2-yl)-2-(quinolin-4-yl)acetamide; N-(4-bromo-3-(1H-1,2,4-triazol-5-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(8-fluoroisoquinolin-5-yl)acetamide; N-(4-bromo-3-(1H-1,2,4-triazol-5-yl)thiophen-2-yl)-2-(2-oxo-1,6-naphthyridin-1(2H)-yl)acetamide; N-(4-bromo-3-(1H-1,2,4-triazol-5-yl)thiophen-2-yl)-2-(8-fluoroquinolin-5-yl)acetamide; N-(4-bromo-3-(1H-1,2,4-triazol-5-yl)thiophen-2-yl)-2-(2-oxo-1,5-naphthyridin-1(2H)-yl)acetamide; N-(4-bromo-3-(1H-1,2,4-triazol-5-yl)thiophen-2-yl)-2-(2-oxo-3,4-dihydro-1-, 6-naphthyridin-1(2H)-yl)acetamide; N-(4-bromo-3-(1H-1,2,4-triazol-5-yl)thiophen-2-yl)-2-(2-oxo-3,4-dihydro-1,5-naphthyridin-1(2H)-yl)acetamide; N-(4-bromo-3-(1H-1,2,4-triazol-5-yl)thiophen-2-yl)-2-(6-chloro-2-oxoquinolin-1(2H)-yl)acetamide; N-(4-bromo-3-(1H-1,2,4-triazol-5-yl)thiophen-2-yl)-2-(5-fluoro-2-oxoquinolin-1(2H)-yl)acetamide; N-(4-bromo-3-(1H-1,2,4-triazol-5-yl)thiophen-2-yl)-2-(2-oxo-6-(trifluoromethoxy)quinolin-1(2H)-yl)acetamide; N-(4-bromo-3-(4H-1,2,4-triazol-3-yl)thiophen-2-yl)-2-(isoquinolin-4-yl)acetamide; N-(4-bromo-3-(1H-1,2,4-triazol-3-yl)thiophen-2-yl)-2-(3-fluoroquinolin-5-yl)acetamide; N-(4-bromo-3-(1H-1,2,4-triazol-5-yl)thiophen-2-yl)-2-(2-oxo-7-(trifluoromethoxy)quinolin-1(2H)-yl)acetamide; N-(4-bromo-3-(1H-1,2,4-triazol-5-yl)thiophen-2-yl)-2-(7-cyano-2-oxoquinolin-1(2H)-yl)acetamide; N-(4-bromo-3-(1H-1,2,4-triazol-5-yl)thiophen-2-yl)-2-(isoquinolin-8-yl)acetamide; N-(4-bromo-3-(1H-1,2,4-triazol-5-yl)thiophen-2-yl)-2-(6-cyano-2-oxoquinolin-1(2H)-yl)acetamide; N-(4-bromo-3-(1H-1,2,4-triazol-5-yl)thiophen-2-yl)-2-(2-oxo-5-(trifluoromethyl)quinolin-1(2H)-yl)acetamide; N-(4-bromo-3-(1H-1,2,4-triazol-5-yl)thiophen-2-yl)-2-(2-oxo-6-(trifluoromethyl)-3,4-dihydroquinolin-1(2H)-yl)acetamide; N-(4-cyano-3-(1-methyl-1H-1,2,4-triazol-3-yl)thiophen-2-yl)-2-(quinolin-5-yl)acetamide; N-(4-bromo-3-(1H-1,2,4-triazol-5-yl)thiophen-2-yl)-2-(7-fluoroquinolin-5-yl)acetamide; N-(4-bromo-3-(1H-1,2,4-triazol-3-yl)thiophen-2-yl)-2-(3-(trifluoromethyl)-quinolin-5-yl)acetamide; N-(4-bromo-3-(1H-1,2,4-triazol-5-yl)thiophen-2-yl)-2-(6-fluoroquinolin-5-yl)acetamide; N-(4-bromo-3-(1H-1,2,4-triazol-5-yl)thiophen-2-yl)-2-(6-ethynyl-2-oxoquinolin-1(2H)-yl)acetamide; N-(4-bromo-3-(1H-1,2,4-triazol-5-yl)thiophen-2-yl)-2-(2-oxo-7-(trifluoromethyl)-1,6-naphthyridin-1(2H)-yl)acetamide; N-(4-bromo-3-(1H-1,2,4-triazol-5-yl)thiophen-2-yl)-2-(5-oxopyrazolo[1,5-a]pyrimidin-4(5H)-yl)acetamide; 2-(2-oxo-6-(trifluoromethyl)quinolin-1(2H)-yl)-N-(2-(thiazol-4-yl)thiophen-3-yl)acetamide; N-(4-cyano-3-(pyrazin-2-yl)thiophen-2-yl)-2-(quinolin-5-yl)acetamide; 2-(benzo[d]thiazol-7-yl)-N-(4-bromo-3-(1H-1,2,4-triazol-5-yl)thiophen-2-yl)acetamide; N-(4-chloro-3-(1H-1,2,4-triazol-5-yl)thiophen-2-yl)-2-(2-oxo-6-(trifluoromethyl)quinolin-1(2H)-yl)acetamide; N-(4-cyano-3-(oxazol-2-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(2-oxo-6-(trifluoromethyl)-1,5-naphthyridin-1(2H)-yl)acetamide; N-(4-cyano-3-(thiazol-2-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(5-oxo-2-(trifluoromethyl)pyrazolo[1,5-a]pyrimidin-4(5H)-yl)acetamide; N-(4-cyano-3-(thiazol-4-yl)thiophen-2-yl)-2-(2-oxo-6-(trifluoromethyl)quinolin-1(2H)-yl)acetamide; N-(4-bromo-3-(1H-1,2,4-triazol-5-yl)thiophen-2-yl)-2-(7-fluoro-2-oxo-6-(trifluoromethyl)quinolin-1(2H)-yl)acetamide; N-(4-bromo-3-(1-methyl-1H-1,2,4-triazol-3-yl)thiophen-2-yl)-2-(2-oxo-3,4-dihydro-1,5-naphthyridin-1(2H)-yl)acetamide; N-(4-bromo-3-(1H-1,2,4-triazol-5-yl)thiophen-2-yl)-2-(6-fluoroisoquinolin-5-yl)acetamide; N-(4-bromo-3-(1H-1,2,4-triazol-5-yl)thiophen-2-yl)-2-(6-fluoroisoquinolin-7-yl)acetamide; N-(4-cyano-3-(2H-1,2,3-triazol-2-yl)thiophen-2-yl)-2-(2-oxo-6-(trifluoromethyl)quinolin-1(2H)-yl)acetamide; N-(4-chloro-3-(1H-1,2,4-triazol-3-yl)thiophen-2-yl)-2-(2-oxo-3,4-dihydro-1,5-naphthyridin-1(2H)-yl)acetamide; N-(4-cyano-3-(thiazol-2-yl)thiophen-2-yl)-2-(2-oxo-3,4-dihydro-1,5-naphthyridin-1(2H)-yl)acetamide; N-(4-chloro-3-(1-methyl-1H-1,2,4-triazol-3-yl)thiophen-2-yl)-2-(2-oxo-3,4-dihydro-1,5-naphthyridin-1(2H)-yl)acetamide; N-(4-chloro-3-(1H-1,2,4-triazol-5-yl)thiophen-2-yl)-2-(5-oxopyrazolo[1,5-a]pyrimidin-4(5H)-yl)acetamide; N-(4-bromo-3-(oxazol-2-yl)thiophen-2-yl)-2-(2-oxo-3,4-dihydro-1,5-naphthyridin-1(2H)-yl)acetamide; N-(4-bromo-3-(thiazol-2-yl)thiophen-2-yl)-2-(2-oxo-3,4-dihydro-1,5-naphthyridin-1(2H)-yl)acetamide; N-(4-chloro-3-(thiazol-2-yl)thiophen-2-yl)-2-(2-oxo-3,4-dihydro-1,5-naphthyridin-1(2H)-yl)acetamide; N-(4-chloro-3-(3-methyl-1H-1,2,4-triazol-5-yl)thiophen-2-yl)-2-(2-oxo-3,4-dihydro-1,5-naphthyridin-1(2H)-yl)acetamide; N-(4-chloro-3-(1-(3-(dimethylamino)propyl)-1H-1,2,4-triazol-3-yl)thiophen-2-yl)-2-(2-oxo-6-(trifluoromethyl)quinolin-1(2H)-yl)acetamide; N-(4-chloro-3-(1-(3-(4-methylpiperazin-1-yl)propyl)-1H-1,2,4-triazol-3-yl-)thiophen-2-yl)-2-(2-oxo-6-(trifluoromethyl)quinolin-1(2H)-yl)acetamide; N-(4-chloro-3-(3-ethyl-1H-1,2,4-triazol-5-yl)thiophen-2-yl)-2-(2-oxo-3,4-dihydro-1,5-naphthyridin-1(2H)-yl)acetamide; N-(4-chloro-3-(1-(2-(dimethylamino)ethyl)-1H-1,2,4-triazol-3-yl)thiophen-2-yl)-2-(2-oxo-6-(trifluoromethyl)quinolin-1(2H)-yl)acetamide; N-(4-cyano-3-(1H-1,2,4-triazol-3-yl)thiophen-2-yl)-2-(2-oxo-3,4-dihydro-1-, 5-naphthyridin-1(2H)-yl)acetamide; N-(4-chloro-3-(1-methyl-1H-1,2,4-triazol-3-yl)thiophen-2-yl)-2-(5-oxopyrazolo[1,5-a]pyrimidin-4(5H)-yl)acetamide; N-(4-chloro-3-(1-(3-morpholinopropyl)-1H-1,2,4-triazol-3-yl)thiophen-2-yl-)-2-(2-oxo-6-(trifluoromethyl)quinolin-1(2H)-yl)acetamide; N-(4-chloro-3-(1-(3-(pyrrolidin-1-yl)propyl)-1H-1,2,4-triazol-3-yl)thiophen-2-yl)-2-(2-oxo-6-(trifluoromethyl)quinolin-1(2H)-yl)acetamide; N-(4-chloro-3-(1-methyl-1H-1,2,4-triazol-3-yl)thiophen-2-yl)-2-(2-oxo-6-(trifluoromethyl)-1,5-naphthyridin-1(2H)-yl)acetamide; N-(4-bromo-3-(2H-1,2,3-triazol-2-yl)thiophen-2-yl)-2-(2-oxo-6-(trifluoromethyl)quinolin-1(2H)-yl)acetamide; N-(4-chloro-3-(1H-1,2,4-triazol-5-yl)thiophen-2-yl)-2-(2-oxo-6-(trifluoromethyl)-1,5-naphthyridin-1(2H)-yl)acetamide; 2-(6-bromo-2-oxoquinolin-1(2H)-yl)-N-(4-bromo-3-(1H-1,2,4-triazol-3-yl)thiophen-2-yl)acetamide; N-(4-cyano-3-(1-methyl-1H-1,2,4-triazol-3-yl)thiophen-2-yl)-2-(2-oxo-3,4-dihydro-1,5-naphthyridin-1(2H)-yl)acetamide; 2-(6-bromo-2-oxoquinolin-1(2H)-yl)-N-(4-chloro-3-(1H-1,2,4-triazol-3-yl)thiophen-2-yl)acetamide; N-(4-chloro-3-(1H-1,2,4-triazol-3-yl)thiophen-2-yl)-2-(6-cyano-2-oxoquinolin-1(2H)-yl)acetamide; N-(4-chloro-3-(1H-1,2,4-triazol-5-yl)thiophen-2-yl)-2-(5-oxo-2-(trifluoromethyl)pyrazolo[1,5-a]pyrimidin-4(5H)-yl)acetamide; N-(4-chloro-3-(1H-1,2,4-triazol-3-yl)thiophen-2-yl)-2-(2-oxo-7-(trifluoromethyl)-1,6-naphthyridin-1(2H)-yl)acetamide; N-(4-chloro-3-(1-methyl-1H-1,2,4-triazol-3-yl)thiophen-2-yl)-2-(5-oxo-2-(trifluoromethyl)pyrazolo[1,5-a]pyrimidin-4(5H)-yl)acetamide; and N-(4-chloro-3-(1-methyl-1H-1,2,4-triazol-3-yl)thiophen-2-yl)-2-(2-oxo-7-(trifluoromethyl)-1,6-naphthyridin-1(2H)-yl)acetamide, or a pharmaceutically acceptable salt thereof.

11. The method of claim 7, wherein the compound according to Formula (I) exhibits an IC₅₀<10 μM.

12. The method of claim 7, wherein the compound according to Formula (I) exhibits an IC₅₀<0.1 μM.
